SEQUENCE LISTING (23 sequences)

SEQ ID NO: 1 - protein, 10 aa, Artificial Sequence
  Synthetic Sequence
GFTFSNHDLN

SEQ ID NO: 2 - protein, 17 aa, Artificial Sequence
  Synthetic Sequence
YISSASGLISYADAVRG

SEQ ID NO: 3 - protein, 12 aa, Artificial Sequence
  Synthetic Sequence
DPAYTGLYALDF

SEQ ID NO: 4 - protein, 12 aa, Artificial Sequence
  Synthetic Sequence
DPPYSGLYALDF

SEQ ID NO: 5 - protein, 12 aa, Artificial Sequence
  Synthetic Sequence
TLSSELSWYTIV

SEQ ID NO: 6 - protein, 11 aa, Artificial Sequence
  Synthetic Sequence
LKSDGSHSKGD

SEQ ID NO: 7 - protein, 14 aa, Artificial Sequence
  Synthetic Sequence
CGAGYTLAGQYGWV

SEQ ID NO: 8 - protein, 121 aa, Artificial Sequence
  Synthetic Sequence
EVQLVESGGGLSQPGNSLQLSCEASGFTFSNHDLNWVRQAPGKGLEWVAYISSASGLISYADAVRGRFTISRDNAKNSLFLQMNNLKSEDTAMYYCARDPPYSGLYALDFWGQGTQVTVSS

SEQ ID NO: 9 - protein, 115 aa, Artificial Sequence
  Synthetic Sequence
QPVLTQSPSASASLSGSVKLTCTLSSELSSYTIVWYQQRPDKAPKYVMYLKSDGSHSKGDGIPDRFSGSSSGAHRYLSISNVQSEDDATYFCGAGYTLAGQYGWVFGSGTKVTVL

SEQ ID NO: 10 - protein, 121 aa, Artificial Sequence; identical to SEQ ID NO: 8
  Synthetic Sequence

SEQ ID NO: 11 - protein, 115 aa, Artificial Sequence; identical to SEQ ID NO: 9
  Synthetic Sequence

SEQ ID NO: 12 - protein, 10 aa, Artificial Sequence
  Synthetic Sequence
GFTFSNHDLN

SEQ ID NO: 13 - protein, 17 aa, Artificial Sequence
  Synthetic Sequence
YISSASGLISYADAVRG

SEQ ID NO: 14 - protein, 12 aa, Artificial Sequence
  Synthetic Sequence
DPPYSGLYALDF

SEQ ID NO: 15 - protein, 12 aa, Artificial Sequence
  Synthetic Sequence
TLSSELSSYTIV

SEQ ID NO: 16 - protein, 11 aa, Artificial Sequence
  Synthetic Sequence
LKSDGSHSKGD

SEQ ID NO: 17 - protein, 13 aa, Artificial Sequence
  Synthetic Sequence
GAGYTLAGQYGWV

SEQ ID NO: 18 - protein, 121 aa, Artificial Sequence
  Synthetic Sequence
EVQLVESGGGLVQPGGSLRLSCEASGFTFSNHDLNWVRQAPGKGLEWVAYISSASGLISYADAVRGRFTISRDNAKNSLYLQMNSLRAEDTAVYYCARDPPYSGLYALDFWGQGTQVTVSS

SEQ ID NO: 19 - protein, 121 aa, Artificial Sequence
  Synthetic Sequence
EVQLVESGGGLVQPGGSLRLSCAASGFTFSNHDLNWVRQAPGKGLEWVAYISSASGLISYADAVRGRFTISRDNAKNSLYLQMNSLRAEDTAVYYCARDPPYSGLYALDFWGQGTQVTVSS

SEQ ID NO: 20 - protein, 121 aa, Artificial Sequence
  Synthetic Sequence
EVQLVESGGGLVQPGGSLRLSCEASGFTFSNHDLNWVRQAPGKGLEWVSYISSASGLISYADAVRGRFTISRDNAKNSLYLQMNSLRAEDTAVYYCARDPPYSGLYALDFWGQGTQVTVSS

SEQ ID NO: 21 - protein, 121 aa, Artificial Sequence
  Synthetic Sequence
EVQLVESGGGLVQPGGSLRLSCAASGFTFSNHDLNWVRQAPGKGLEWVSYISSASGLISYADAVRGRFTISRDNAKNSLYLQMNSLRAEDTAVYYCARDPPYSGLYALDFWGQGTQVTVSS

SEQ ID NO: 22 - protein, 115 aa, Artificial Sequence
  Synthetic Sequence
QPVLTQSSSASASLGSSVKLTCTLSSELSSYTIVWHQQQPGKAPRYLMYLKSDGSHSKGDGVPDRFSGSSSGADRYLTISNLQSEDEADYYCGAGYTLAGQYGWVFGSGTKVTVL

SEQ ID NO: 23 - protein, 115 aa, Artificial Sequence
  Synthetic Sequence
QLVLTQSPSASASLGASVKLTCTLSSELSSYTIVWHQQQPEKGPRYLMYLKSDGSHSKGDGIPDRFSGSSSGAERYLTISSLQSEDEADYYCGAGYTLAGQYGWVFGSGTKVTVL

(19) United States
(12) Patent Application Publication
(10) Pub. No.: US 2022/0340672 A1
(43) Pub. Date: Oct. 27, 2022

(54) TNFRSF25-MEDIATED TREATMENTS OF IMMUNE DISEASES AND DISORDERS

(71) Applicants: Pelican Therapeutics, Inc., Durham, NC (US); The Governors of the University of Alberta, Edmonton (CA)

(72) Inventors: Matthew M. SEAVEY, Durham, NC (US); Jeff T. HUTCHINS, Claremore, OK (US); Rahul R. JASUJA, Durham, NC (US); A.M. James SHAPIRO, Edmonton (CA); Braulio A. MARFIL-GARZA, Edmonton (CA)

(21) Appl. No.: 17/763,430

(22) PCT Filed: Sep. 28, 2020
(86) PCT No.: PCT/US20/53085

§ 371 Date: Mar. 24, 2022

Related U.S. Application Data
(60) Provisional application No. 62/906,438, filed on Sep. 26, 2019.

(51) Int. Cl. C07K 16/28; A61P 3/10; A61K 39/395; A61K 38/28; A61P 37/06; A61K 38/20

(52) U.S. Cl. CPC C07K 16/2878; A61P 3/10; A61K 39/3955; A61K 38/28; A61P 37/06; A61K 38/2013; A61K 2039/505

(57) ABSTRACT

The present disclosure is directed to a method of treating or preventing diabetes, prediabetes, and/or glucose intolerance using TNF Receptor Superfamily Member 25 (TNFRSF25) agonistic antibody or antigen binding fragment thereof. The disclosure is also directed to methods for increasing graft survival and for treating or preventing graft-versus-host disease (GVHD) using TNFRSF25 agonistic antibody.

### CROSS-REFERENCE TO RELATED APPLICATIONS

This application claims the priority and benefit of U.S. Provisional Patent Application No. 62/906,438, filed Sep. 26, 2019, the disclosure of which is hereby incorporated by reference in its entirety.

### FIELD

The disclosure is directed to treatment and prevention of immune-related diseases and disorders such as diabetes and related conditions and graft-versus host disease, using TNF Receptor Superfamily Member 25 (TNFRSF25) agonistic antibodies.

### SEQUENCE LISTING

The application contains a Sequence Listing which has been submitted in ASCII format via EFS-Web and is hereby incorporated by reference in its entirety. Said ASCII copy, created on Sep. 28, 2020, is named PEL-016PC_ST25.txt and is 14,496 bytes in size.

### BACKGROUND

Tregs are T cells which have a role in regulating or suppressing other cells in the immune system. Stimulation of tumor necrosis factor receptor superfamily, member 25 (TNFRSF25), in vivo facilitates selective proliferation of Tregs in mice and suppression of immunopathology in allergic lung inflammation, allogeneic heart transplantation and HSV-1 mediated ocular inflammation.

Various immune-related diseases and disorders remain major health problems and, among the most pressing medical needs are treatments for diabetes and graft-versus-host disease (GVHD). Diabetes affects a large number of people in the United States and abroad, and many new cases occur each year. Diabetes is linked to a number of health problems, including microvascular complications, such as retinopathy, neuropathy, and nephropathy. Further, in the United States, diabetes is the leading cause of new blindness in working-age adults, new cases of end-stage renal disease, and non-traumatic lower leg amputations. In addition, cardiovascular complications are now the leading cause of diabetes-related morbidity and mortality, particularly among women and the elderly. In adult patients with diabetes, the risk of cardiovascular disease (CVD) is three-to-five fold greater than in the general population.

Islet cell transplantation has been recognized as an emerging, promising therapeutic approach to treatment of diabetes. However, immunosuppression is required to prevent host rejection of donor islet cells, as is common for allografts in human organ transplantation. Moreover, islet cell transplantation can be complicated by allograft rejection and GVHD, a common immune response to donor's cells in the context of a transplant which can lead to, among other issues, serious immune system complications and even death.

Current therapies for diabetes and related diseases suffer from inadequate responses and/or patient adherence.

Therefore, there remains a need for more effective and accessible methods of treating diabetes, allograft rejection and/or GVHD.

### SUMMARY

Accordingly, the present invention provides new methods and uses for the treatment and/or prevention of diabetes, including, for example, type 1 and type 2 diabetes, and related diseases (e.g., prediabetes and glucose intolerance), comprising administering an effective amount of TNF Receptor Superfamily Member 25 (TNFRSF25) agonistic antibody or antigen binding fragment thereof to a patient in need thereof. The treatment may expand and/or selectively activate a population of Tregs in the patient.

The described methods can be used to treat and/or prevent diabetes and other related conditions in patients suffering other conditions and/or diseases. For example, the patient may be diagnosed with one or more of insulin resistance, prediabetes, impaired fasting glucose (IFG), impaired glucose tolerance (IGT), and acanthosis nigricans. The patient may have cardiovascular disease or metabolic disease, type 1 diabetes or type 2 diabetes, gestational diabetes or steroid-induced diabetes, or other conditions or diseases.

In embodiments, the patient may be undergoing treatment with one or more of insulin or an insulin analog. The insulin analog can be selected from a rapid acting insulin analog (e.g., lispro, aspart or glulisine) or a long acting insulin analog (e.g., glargine or detemir).

In embodiments, a method for treating diabetes and/or glucose intolerance is provided that comprises administering an effective amount of TNFRSF25 agonistic antibody or antigen binding fragment thereof to a patient in need thereof, wherein the patient is not receiving insulin therapy. In such embodiments, the TNFRSF25 agonistic antibody or antigen binding fragment can stimulate glucose uptake in the patient.

In some aspects, the present invention also provides a method for increasing a graft or transplant survival and preventing or delaying a graft or transplant rejection. The graft, interchangeably referred to herein as a transplant, can be a solid organ graft (e.g., pancreas or other organ). In some embodiments, the graft can comprise islet cells which can be obtained from either a donor (islet allo-transplantation) or a recipient (islet auto-transplantation).

In some embodiments, the administration of TNFRSF25 agonistic antibody or antigen binding fragment thereof to a patient in need thereof results in a Treg expansion. The Treg expansion can include expansion of FoxP3+ cells out of CD4+ T cells, and/or expansion of one or both CD4+FOXP3+ T cells and CD4+CD25+FOXP3+ T cells in the patient. In embodiments in which the patient, which can be afflicted by diabetes or similar disease(s), is a transplant recipient (e.g., a recipient of an islet cells graft, pancreatic graft, etc), the increase in the Treg expansion can result in increase in a graft survival and prevention or delay of a graft rejection and GVHD.

In some embodiments, a method for treating or preventing graft-versus-host disease (GVHD) is provided, comprising administering an effective amount of TNFRSF25 agonistic antibody or antigen binding fragment thereof to a patient in need thereof. The patient may be a transplant recipient, and the transplant recipient may receive the transplant comprising islet cells from a transplant donor. In some embodiments, the transplant may comprise donor T cells, donor hematopoietic cells, donor stem cells, or donor bone marrow cells. The method may involve reducing a graft versus host disease (GVHD), which can be acute graft-versus-host-disease (aGVHD) or chronic graft-versus-host-disease (cGVHD).

In some embodiments, a method for treating or preventing GVHD is provided, comprising administering an effective amount of TNF Receptor Superfamily Member 25 (TNFRSF25) agonistic antibody or antigen binding fragment thereof to a patient in need thereof. The patient may be a transplant recipient, and the transplant recipient may receive the transplant comprising islet cells from a transplant donor. The transplant may comprise donor T regulatory cell, donor hematopoietic cells, donor stem cells, or donor bone marrow cells. The method may involve reducing a graft versus host disease (GVHD), which can be acute graft-versus-host-disease (aGVHD) or chronic graft-versus-host-disease (cGVHD).

The administration of the effective amount of TNFRSF25 agonistic antibody or antigen binding fragment can be to a transplant donor. In some embodiments, the administration to the transplant donor can occur prior to transplant. In some embodiments, the administration is to both the transplant donor and transplant recipient. In some embodiments, the method prevents a transplant rejection, e.g., a solid organ transplant rejection, islet cell transplant rejection, or a stem cell transplant rejection.

In some embodiments, TNFRSF25 agonistic antibody or antigen binding fragment thereof comprises (i) a heavy chain variable region comprising heavy chain CDR1, CDR2, and CDR3 sequences, wherein the heavy chain CDR1 sequence is GFTFSNHDLN (SEQ ID NO: 1), the heavy chain CDR2 sequence is YISSASGLISYADAVRG (SEQ ID NO: 2); and the heavy chain CDR3 sequence is DPAYTGLYALDF (SEQ ID NO: 3) or DPPYSGLYALDF (SEQ ID NO: 4); and (ii) a light chain variable region comprising light chain CDR1, CDR2, and CDR3 sequences, wherein the light chain CDR1 sequence is TLSSELSWYTIV (SEQ ID NO: 5), the light chain CDR2 sequence is LKSDGSHSKGD (SEQ ID NO: 6), and the light chain CDR3 sequence is CGAGYTLAGQYGWV (SEQ ID NO: 7).

In some embodiments, the TNFRSF25 agonistic antibody or antigen binding fragment thereof further comprises variable region framework (FW) sequences juxtaposed between the CDRs according to the formula (FW1)-(CDR1)-(FW2)-(CDR2)-(FW3)-(CDR3)-(FW4), wherein the variable region FW sequences in the heavy chain variable region are heavy chain variable region FW sequences, and wherein the variable region FW sequences in the light chain variable region are light chain variable region FW sequences. The variable region FW sequences may be human.

In some embodiments, the TNFRSF25 agonistic antibody or antigen binding fragment thereof further comprises human heavy chain and light chain constant regions. The constant regions can be selected from the group consisting of human IgG1, IgG2, IgG3, and IgG4. For example, the constant regions can be IgG1 or IgG4.

In some embodiments, the TNFRSF25 agonistic antibody or antigen binding fragment thereof comprises a heavy chain variable region having the amino acid sequence EVQLVESGGGLSQPGNSLQLSCEASGFTFSNHDLNWVRQAPGKGLEWVAYISSASGLISYADAVRGRFTISRDN AKNSLFLQMNNLKSEDTAMYYCARDPPYSGLYALDFWGQGTQVTVSS (SEQ ID NO: 8), or an amino acid sequence of at least about 85 to about 99% identity thereto. In some embodiments, the TNFRSF25 agonistic antibody or antigen binding fragment thereof comprises a light chain variable region having the amino acid sequence QPVLTQSPSASASLSGSVKLTCTLSSELSSYTIVWYQQRPDKAPKYVMYLKSDGSHSKGDGIPDRFSGSSSGAH RYLSISNVQSEDDATYFCGAGYTLAGQYGWVFGSGTKVTVL (SEQ ID NO: 9), or an amino acid sequence of at least about 85 to about 99% identity thereto.

### BRIEF DESCRIPTION OF THE DRAWINGS

FIG. 1 is a non-limiting schematic illustrating a design of in vivo experiments on expansion of regulatory T cells in islet transplantation to determine the role of TNFRSF25 in the expansion of the size of the Treg population in vivo. Four groups of mice (n=2 per group) were injected with saline (control), streptozotocin (STZ), mouse anti-TNFRSF25 agonist (“Anti-TNFRSF25 agonist”), and combination of STZ+anti-TNFRSF25 agonist (“STZ+Anti-TNFRSF25 agonist”). STZ dose was 175 mg/kg, and anti-TNFRSF25 agonist dose was 10 mg/kg. Blood flow cytometry was performed to assess the Treg response, days 0-31. On day 22, the mice (n=2) were injected the second dose of anti-TNFRSF25 agonist, and CD4+/FoxP3+ regulatory T-cells were monitored through time (days 0-9).

FIG. 2 are graphs illustrating Tregs kinetics through time for the mice of the study of FIG. 1. FoxP3+ cells (% of CD4+ T cells) are shown for days 0-22.

FIG. 3 are graphs illustrating comparison of Tregs kinetics through time for the mice of the study of FIG. 1. FoxP3+ cells (% of CD4+ T cells) are shown for days 22-31 after the first anti-TNFRSF25 agonist injection and respectively days 0-9 after the second anti-TNFRSF25 agonist injection. (*—no data was obtained from 28(6) time points).

FIG. 4 are graphs illustrating results of the study of the effect of TNFRSF25 antibody in a murine model of islet transplantation. The results are shown for three mice (panels A, B, and C) on day 6. FoxP3+CD4+ T cells expansion is shown in the following analyzed groups (in panels A and B): mouse PTX-35 IgG1 (“msPTX-35 IgG1”), IgG1, msPTX-35 IgG2, IgG2, human PTX-35 lot C (“HuPTX35_lotC”), and 4C12. In panel C, the analyzed groups are as follows: IgG1 (“Isotype IgG1”), IgG2 (“Isotype IgG2a”), 4C12, mouse PTX-35 IgG1 (“SR (surrogate) PTX-35 IgG2a”), and human PTX-35 lot C (“Hum PTX-35 Lot C”). In each of the panels A to C, the top graph shows flow cytometry data and the bottom chart illustrates percentage of FoxP3+CD4+ T regs.

FIG. 5 are graphs illustrating CD4+/FoxP3+ cells (% of CD4+/CD3+ cells) in a murine on days 0-9, in the group administered mPTX-35 (n=2), the group administered STZ (n=2), and in the control group (n=2).

FIG. 6 are graphs illustrating flow cytometry gating data for the control group (left panel, n=2) and the group administered mPTX-35 IgG1 (right panel, n=2). In both panels, the top graph shows CD3+CD4+FoxP3+ on day 0 of the study, and the bottom graph shows CD3+CD4+FoxP3+ on day 6 of the study.

FIG. 7 are graphs illustrating CD3+/CD4+ cells (% of total cells) on days 0 through 9, in the group administered mPTX-35 (n=2), the group administered STZ (n=2), and in the control group (n=2).

FIG. 8 are graphs illustrating CD25+/FoxP3+ cells (% of CD4+/CD3+ cells) in the group administered mPTX-35 (n=2), the group administered STZ (n=2), and in the control group (n=2).

FIG. 9 is a non-limiting schematic illustrating a design of experiments to determine the role of an anti-TNFRSF25 monoclonal chimeric (mouse-human) agonistic antibody (4C12) in islet transplantation in vivo.

FIG. 10 are graphs illustrating flow cytometry gating data for CD3+/CD4+ cells (% of total cells) on day -4 (left panel), and day -0 (right panel), in the murine group administered saline (control; top panels), and the murine group that had the 4C12 antibody administered (bottom panels).

FIG. 11A-FIG. 11L are graphs illustrating the effect of the 4C12 antibody in a murine model of islet transplantation. FIGS. 11A, 11B, and 11C illustrate results as CD4+FoxP3+ cells (a percentage of lymphocytes), where the results were obtained using fifteen control mice (n=15) and 19 4C12 experimental mice (n=19). In FIG. 11A, CD4+FoxP3+ cells are shown as a percentage of lymphocytes at day -4 (“D -4”) from transplant (control experimental group, left bar; 4C12 experimental group, right bar), and at day 0 (“DO”) of transplant (control experimental group, left bar; 4C12 experimental group, right bar. In FIG. 11B, the increase in CD4+FoxP3+ cells is shown as a percentage of lymphocytes at day -4 from transplant, and at day 0 of transplant. In FIG. 11C, the fold increase (“DO” from transplant/“D-4” from transplant) in CD4+FoxP3+ cells as a percentage of lymphocytes is shown (control experimental group, left bar; 4C12 experimental group, right bar). FIGS. 11D, 11E, and 11F illustrate results as FoxP3+ cells (a percentage of CD4+ T cells), where the results were obtained using fifteen control mice (n=15) and 19 4C12 experimental mice (n=19). In FIG. 11D, FoxP3+ cells are shown as a percentage of CD4+ T cells at day -4 from transplant (control experimental group, left bar; 4C12 experimental group, right bar), and at day 0 of transplant (control experimental group, left bar; 4C12 experimental group, right bar). In FIG. 11E, the increase in FoxP3+ cells is shown as a percentage of CD4+ T cells at day -4 from transplant, and at day 0 of transplant. In FIG. 11F, the fold increase (“DO” from transplant/“D-4” from transplant) in FoxP3+ cells as a percentage of CD4+ T cells is shown (control experimental group, left bar; 4C12 experimental group, right bar). FIGS. 11G, 11H, and 11I illustrate results as CD25+FoxP3+ cells (a percentage of lymphocytes), where the results were obtained using twenty-six control mice (n=26) and 33 4C12 experimental mice (n=33). In FIG. 11G, CD25+FoxP3+ cells are shown as a percentage of lymphocytes at day -4 from transplant (control experimental group, left bar; 4C12 experimental group, right bar), and at day 0 of transplant (control experimental group, left bar; 4C12 experimental group, right bar). In FIG. 11H, the increase in CD25+FoxP3+ cells is shown as a percentage of lymphocytes at day -4 from transplant, and at day 0 of transplant. In FIG. 11I, the fold increase (“DO” from transplant/“D-4” from transplant) in CD25+FoxP3+ cells as a percentage of lymphocytes is shown (control experimental group, left bar; 4C12 experimental group, right bar). FIGS. 11J, 11K, and 11L illustrate results as CD25+FoxP3+ cells (a percentage of CD4+ T cells), where the results were obtained using fifteen control mice (n=15) and 19 4C12 experimental mice (n=19). In FIG. 11J, CD25+FoxP3+ cells are shown as a percentage of CD4+ T cells at day -4 from transplant (control experimental group, left bar; 4C12 experimental group, right bar), and at day 0 of transplant (control experimental group, left bar; 4C12 experimental group, right bar). In FIG. 11K, the increase in CD25+FoxP3+ cells is shown as a percentage of CD4+ T cells at day -4 from transplant, and at day 0 of transplant. In FIG. 11L, the fold increase (“DO” from transplant/“D-4” from transplant) in CD25+FoxP3+ cells as a percentage of CD4+ T cells is shown (control experimental group, left bar; 4C12 experimental group, right bar).

FIG. 12A -FIG. 12I are graphs illustrating the effect of administering STZ with the control group, and administering STZ with 4C12 in the experimental group in a murine model of islet transplantation. Twenty-six control mice (n=26) and 33 4C12 experimental mice (n=33) were used in this study, and STZ was administered to each group of mice. The control group of mice received saline and STZ, and the experimental group of mice received the 4C12 antibody and STZ. In FIG. 12A, T cells are shown as a percentage of lymphocytes at day -4 from transplant (control experimental group, left bar; 4C12 experimental group, right bar), and at day 0 of transplant (control experimental group, left bar; 4C12 experimental group, right bar). In FIG. 12B, the decrease in T cells is shown as a percentage of lymphocytes at day -4 from transplant, and at day 0 of transplant, In FIG. 12C, the fold increase (“DO” from transplant/“D-4” from transplant) in T cells as a percentage of lymphocytes is shown (control experimental group, left bar; 4C12 experimental group, right bar). In FIG. 12D, CD4+ T cells are shown as a percentage of lymphocytes at day -4 from transplant (control experimental group, left bar; 4C12 experimental group, right bar), and at day 0 of transplant (control experimental group, left bar; 4C12 experimental group, right bar). In FIG. 12E, the decrease CD4+ T cells are shown as a percentage of lymphocytes at day -4 from transplant, and at day 0 of transplant. In FIG. 12F, the fold increase (“DO” from transplant/D-4” from transplant) in CD4+ T cells as a percentage of lymphocytes is shown (control experimental group, left bar; 4C12 experimental group, right bar). In FIG. 12G, CD3+CD4− cells are shown as a percentage of lymphocytes at day -4 from transplant (control experimental group, left bar; 4C12 experimental group, right bar), and at day 0 of transplant (control experimental group, left bar; 4C12 experimental group, right bar). In FIG. 12H, the decrease in CD3+CD4− cells is shown as a percentage of lymphocytes at day -4 from transplant, and at day 0 of transplant.

In FIG. 12I, the fold increase (“DO” from transplant/“D-4” from transplant) in CD3+CD4− cells as a percentage of lymphocytes is shown (control experimental group, left bar; 4C12 experimental group, right bar).

FIGS. 13A, 13B, and 13C are graphs illustrating effect of 4C12 on Tregs, as Treg curves at different time points; the second 4C12 injection is shown, at day 21 after transplant delivery (injection). The results are shown for 4C12 group (n=3), Isotype control group (n=4), and STZ Isotype control group (n=4). FIG. 13A illustrates the effect of 4C12 on percentage of CD4+FoxP3+ T cells (% of lymphocytes) vs. days after injection.

FIG. 13B illustrates the effect of 4C12 on percentage of FoxP3+ T cells (% of CD4 T cells) vs. days after injection. FIG. 13C illustrates the effect of 4C12 on CD4+FoxP3+ T cells (absolute number of T cells per microliter) vs. days after injection.

FIGS. 14A and 14B illustrate effect of 4C12 on graft survival, showing delayed acute allograft rejection in mice administered 4C12 (n=19) as compared to control mice (n=15). FIG. 14A shows Kaplan-Meier graft survival estimates, for a percent of euglycemic mice over 40 days after transplant, where the mice in the control group had a median graft survival time (“MST”) of 14 days, as compared to the mice treated with 4C12 that had a MST of 19 days (log-rank test, p value=0.0027). FIG. 14B shows a concentration of glucose (mmol/L) over 40 days after transplant.

FIG. 15A − FIG. 15D are graphs showing correlation between Treg expansions and islet graft survival (in days), illustrating that the Treg fold increase has a moderate correlation with graft survival, a fitted values curve is shown. FIG. 15A shows the fold increase of FoxP3+ cells from CD4+ T cells for mice that had the 4C12 antibody administered compared to control mice (Pearson's r=0.49, p=0.003). FIG. 15B shows the fold increase of FoxP3+ cells from CD4+ T cells for the mice that had the 4C12 antibody administered compared to the control mice (Pearson's r=0.57, p=0.0005). FIG. 15C shows the fold increase of CD25+FoxP3+ cells from CD4+ T cells for the mice that had the 4C12 antibody administered compared to the control mice (Pearson's r=0.52, p=0.002).

FIG. 15D shows the fold increase of CD25+FoxP3+ cells from CD4+ T cells for the mice that had the 4C12 antibody administered compared to the control mice (Pearson's r=0.63, p=0.0001).

FIG. 16A − FIG. 16H are graphs showing the effect of 4C12 on levels of several cytokines and biomarkers early after transplant, in picograms per milliliter (pg/mL). The levels of IFN-γ (FIG. 16A), TNF-α (FIG. 16B), IL-1β (FIG. 16C), IL-5 (FIG. 16D), IL-2 (FIG. 16E), IL-10 (FIG. 16F), KC GRO (FIG. 16G), and IL-6 (FIG. 16H) are shown (control experimental group, left bar; 4C12 experimental group, right bar).

FIGS. 17A and 17B illustrate results obtained using immunohistochemistry analysis of Treg infiltration within the islet graft, in an acute graft rejection study. FIG. 17A shows immunohistochemistry for 4C12 mice (left panel) and control mice (right panel). FIG. 17B shows a percentage of FoxP3+ cells out of total cells in the graft, the results are shown for the control mice group (n=6) and for mice administered 4C12 (n=5).

FIG. 18A-FIG. 18D are bar charts illustrating Treg percentages at the day of rejection, for a control mice group (n=15) and for a mice group administered 4C12 (n=18). FIG. 18A shows FoxP3+ cells (% of lymphocytes) for the control mice group (left bar) and for the mice group administered 4C12 (right bar). FIG. 18B shows FoxP3+ cells (% of CD4+ T cells) for the control mice group (left bar) and for the mice group administered 4C12 (right bar). FIG. 18C shows CD25+FoxP3+ cells (% of lymphocytes) for the control mice group (left bar) and for the mice group administered 4C12 (right bar). FIG. 18D shows CD25+FoxP3+ cells (% of CD4+ T cells) for the control mice group (left bar) and for the mice group administered 4C12 (right bar).

FIGS. 19A -FIG. 19D illustrate effect of 4C12 and mPTX-35 on in vivo Treg expansion. mPTX-35 was administered four days before transplantation, concomitantly with STZ, which led to a significant expansion of the Treg populations, including both CD4+FoxP3+ populations and CD4+CD25+FoxP3+(double positive populations). The results were obtained using control experimental mice group (n=20), 4C12 experimental mice group (n=21), and mPTX-35 experimental mice group (n=15). FIG. 19A shows FoxP3+ cells as a percentage of CD4+ cells at day -4 (“D -4”) from transplant (control experimental group, left bar; 4C12 experimental group, middle bar; mPTX35 experimental group, right bar), and at day 0 (“DO”) of transplant (control experimental group, left bar; 4C12 experimental group, middle bar; mPTX-35 experimental group, right bar). FIG. 19B shows the fold increase (“DO” from transplant/“D-4” from transplant) in FoxP3+ cells as a percentage of CD4+ cells (control experimental group, left bar; 4C12 experimental group, middle bar; mPTX-35 experimental group, right bar). FIG. 19C shows CD25+FoxP3+ cells as a percentage of CD4+ cells at day -4 (“D -4”) from transplant (control experimental group, left bar; 4C12 experimental group, middle bar; mPTX35 experimental group, right bar), and at day 0 (“DO”) of transplant (control experimental group, left bar; 4C12 experimental group, middle bar; mPTX-35 experimental group, right bar). FIG. 19D shows the fold increase (“DO” from transplant/“D-4” from transplant) in CD25+FoxP3+ cells as a percentage of CD4+ cells (control experimental group, left bar; 4C12 experimental group, middle bar; mPTX-35 experimental group, right bar).

FIG. 20A -FIG. 20C show results of flow cytometry analysis at different time points to characterize the kinetics of the Treg response in diabetic mice, for 4C12 group (n=3), Isotype control group (n=4), STZ Isotype control group (n=4), PTX-35 group (0.9 mg/kg, n=4), and PTX-35 group (9 mg/kg, n=4). In this study, mPTX-35 was administered four days before transplantation, concomitantly with STZ. The second 4C12 and mPTX-35 injections were administered at day 21 post-transplant delivery (injection). FIG. 20A shows graphs illustrating CD4+FoxP3+ T cells as a percentage of lymphocytes versus a number of days after the transplant delivery (injection). FIG. 20B shows graphs illustrating FoxP3+ T cells as a percentage of CD4+ T cells versus a number of days after the transplant delivery (injection). FIG. 20C shows graphs illustrating CD4+FoxP3+ T cells, as the absolute number of T cells per microliter versus a number of days after the transplant delivery (injection).

FIG. 21 is a graph illustrating a Treg curve showing a 2^{nd} peak of Treg expansion. The graph shows FoxP3+ cells as a percentage of CD4+ cells versus a number of days after the first injection, for non-diabetic mice (n=2) administered mPTX-35 (1 mg/kg).

FIG. 22 shows Kaplan-Meier graft survival estimates, as a percent of euglycemic mice versus a number of days after the transplant), demonstrating that mPTX-35 administration results in a statistically significant delay in acute rejection thereby increasing graft survival. The results are shown for a control mice group (n=19), a mice group administered 4C12 (n=21), and a mice group administered mPTX-35 group (n=7). Mice in the control group had a median graft survival (MST) of 15 days, as compared to the mice group administered 4C12 (MST of 19 days, log-rank test, p=0.001) and the mice group administered mPTX-35 (MST of 17 days, log-rank test, p=0.06).

### DETAILED DESCRIPTION OF THE DISCLOSURE

The present invention is based, in part, on the discovery of surprising immune-modulatory effects of anti-TNFRSF25 agonistic antibodies (e.g., PTX-35). For instance, the present inventors have discovered that anti-TNFRSF25 agonistic antibodies (e.g., PTX-35) cause expansion of Tregs in vivo. The described treatments may expand and/or selectively activate a population of Tregs in the patient. This effect can find use in treating or preventing diabetes, prediabetes, and/or glucose intolerance in patients who may or who may not be receiving insulin therapy. Also, the expansion of Tregs in vivo can be used in treating or preventing a graft rejection, increasing graft survival, and treating or preventing a graft-versus-host disease (GVHD).

The present invention makes use of antibodies targeted to particular epitopes within tumor necrosis factor receptor superfamily member 25 (TNFRSF25). TNFRSF25 is a TNF-receptor superfamily member that is preferentially expressed by activated and antigen-experienced T lymphocytes. The structural organization of the TNFRSF25 protein is most homologous to TNF receptor 1 (TNFR1). The extracellular domain of TNFRSF25 includes four cysteine-rich domains, and the cytoplasmic region contains a death domain known to signal apoptosis. Alternative splicing of the TNFRSF25 gene in B and T cells encounters a program change upon T cell activation, which predominantly produces full-length, membrane bound isoforms, and is involved in controlling lymphocyte proliferation induced by T cell activation. TNFRSF25 is activated by its ligand, TNF-like protein 1A (TL1A), also referred to as TNFSF15, which is rapidly upregulated in antigen presenting cells and in some endothelial cells following Toll-Like Receptor or Fc receptor activation. TL1A has co-stimulatory activity for TNFRSF25-expressing T cells through the activation of NF-κB and suppression of apoptosis by up-regulation of c-IAP2. TNFRSF25 signaling increases the sensitivity of T cells to endogenous IL-2, and enhances T cell proliferation.

The inventors have previously demonstrated that TNFRSF25 is a potent T cell costimulator due to its specificity for expansion of memory CD4+ and CD8+ T cells that are known to maintain tolerance to self antigens, help establish tolerance to allogeneic grafts, suppress differentiation of naïve T cells into effector T cells, and suppress activity of already differentiated T cells. Profileration of T cells (e.g., human T cells, murine T cells, or macaque T cells) can be stimulated by administering an amount of an anti-TNFRSF25 antibody.

In some embodiments, the TNFRSF25 agonistic antibody is PTX-35. PTX-35 is defined by its variable heavy and variable light chain sequences.

Islet transplantation is a method of implantation of pancreatic islets for the treatment of type 1 diabetes mellitus, and it is referred to as Edmonton Protocol established in 2000. See Shapiro et al. (2000) Islet transplantation in seven patients with type 1 diabetes mellitus using a glucocorticoid-free immunosuppressive regimen. The New England Journal of Medicine. 343 (4): 230-238. Islet transplantation requires simultaneous and constant immunosuppression to avoid allograft rejection. See Bottino et al. (2002) Pancreas and islet cell transplantation. Best Pract Res Clin Gastroenterol. 16(3):457-474. Islet transplantation normalizes glucose levels in patients with type 1 diabetes and alleviates hypoglycemic episodes associated with traditional insulin therapy. However, the associated challenge is that islet transplantation requires lifelong immunosuppression, and ways to develop a long term insulin independence have not been developed.

A delivery of a transplant, such as, e.g., islet cell transplantation, to a patient in need thereof using existing approaches is typically plagued by a loss of the function of the transplant, which can be caused by allogeneic rejection, recurrence of autoimmunity, and immunosuppressive drug toxicity. See Wang et al.

Radiology vol. 266, 3 (2013): 822-30. The progressive decline in graft function is observed in many islet recipients. See Forbes et al. American Journal of Transplantation. 2016: 16 (91): pages 2704-2713 (first published: 28 Mar. 2016). Accordingly, the long-term survival and function of islet grafts presents a significant challenge. The present disclosure therefore provides a method that comprises administering an effective amount of TNFRSF25 agonistic antibody or antigen binding fragment thereof to a patient in need thereof to cause Treg expansion and thereby prolong transplant (such as, without limitation or antigen binding fragment thereof, an islet cell transplant) survival. The transplant survival is prolonged such that the transplant remains functional and is not rejected by the recipient's body for a longer period of time as compared to survival of a transplant in patient which was not administered a TNFRSF25 agonistic antibody or antigen binding fragment thereof. The patient may be diagnosed with one or more of insulin resistance, prediabetes, diabetes (type I or type II), impaired fasting glucose (IFG), impaired glucose tolerance (IGT), and acanthosis nigricans.

Accordingly, the present disclosure provides methods for expanding the population of T regulatory cells, e.g., FoxP3+ cells out of CD4+ T cells, which demonstrates that administration of anti-TNFRSF25 agonistic antibodies (e.g., PTX-35) can lead to immunological tolerance, which can prevent the use of chronic immunosuppression in islet transplantation. In embodiments, the present methods relate to a method of islet transplantation in which the present anti-TNFRSF25 agonistic antibodies (e.g., PTX-35) are administered simultaneously with the islet transplantation and/or as a maintenance therapy subsequent to the islet transplantation.

In some embodiments, the expansion of T regulatory cells comprises increase in FoxP3+ cells out of CD4+ T cells, increase in CD4+FOXP3+, and/or increase in CD4+CD25+FOXP3+ cells. In some embodiments, the increase in FoxP3+ cells out of CD4+ T cells is an increase at which at least about 10% of CD4+ T-cells are FOXP3+Tregs. In some embodiments, the increase in FoxP3+ cells out of CD4+ T cells is an increase at which at least about 11%, or at least about 12%, or at least about 13%, or at least about 14%, or at least about 15%, or at least about 16%, or at least about 17%, or at least about 18%, or at least about 19%, or at least about 20% of CD4+ T-cells are FOXP3+Tregs. In some embodiments, the increase in FoxP3+ cells out of CD4+ T cells is an increase at which at least about 25%, or at least about 30%, or at least about 35%, or at least about 40% of CD4+ T-cells are FOXP3+Tregs.

In some embodiments, administration of an anti-TNFRSF25 agonistic antibody in accordance with the present disclosure results in significant Treg expansion in a subject. In some embodiments, the subject is a diabetic patient. The expansion of the Treg populations comprises expansion of CD4+FOXP3+ and/or CD4+CD25+FOXP3+ cells. In some embodiments, the anti-TNFRSF25 agonistic antibody comprises PTX-35 or an antigen binding fragment thereof. In some embodiments, the anti-TNFRSF25 agonistic antibody comprises PTX-25 or an antigen binding fragment thereof.

In some embodiments, an anti-TNFRSF25 agonistic antibody comprises a suitable anti-TNFRSF25 agonistic antibody. In embodiments, an anti-TNFRSF25 agonistic antibody, also referred to an TNFRSF25 agonist (or “DR3”) refers to a substance that binds to the TNFRSF25 receptor and triggers a response in the cell on which the TNFRSF25 receptor is expressed, similar to a response that would be observed by exposing the cell to a natural TNFRSF25 ligand, e.g., TL1A. An agonist is the opposite of an antagonist in the sense that while an antagonist may also bind to the receptor, it fails to activate the receptor and actually completely or partially blocks it from activation by endogenous or exogenous agonists. A partial agonist activates a receptor but does not cause as much of a physiological change as docs a full agonist. Alternatively, another example of a TNFRSF25 agonist is an antibody that is capable of binding and activating TNFRSF25. An example of an anti-TNFRSF25 antibody is 4C12 (agonist). (Deposited under the Budapest Treaty on Behalf of: University of Miami; Date of Receipt of seeds/strain(s) by the ATCC®: May 5, 2009; ATCC® Patent Deposit Designation: PTA-10000. Identification Reference by Depositor: Hybridoma cell line; 4C12; The deposit was tested Jun. 4, 2009 and on that date, the seeds/strain(s) were viable. International Depository Authority: American Type Culture Collection (ATCC®), Manassas, Va., USA). In some embodiments, an anti-TNFRSF25 agonistic antibody is PTX-35, which is shown by the inventors to have an activity similar to that of 4C12.

In embodiments, an anti-TNFRSF25 agonistic antibody is from a genus of anti-TNFRSF25 agonistic antibodies that bind to the TNFRSF25 receptor and trigger a response in the cell on which the TNFRSF25 receptor is expressed. The genus comprises 4C12, PTX-25, PTX-35 (such as mPTX-35 or a humanized PTX-35), 11H08 anti-TNFRSF25 agonistic antibody (see U.S. Patent Application No. 2012/0014950), anti-DR3 mouse monoclonal antibody F05 (see Wen et al., The Journal of Biological Chemistry, 2003; 278:39251-39258), another anti-DR3 monoclonal antibody (see U.S. Pat. No. 7,357,927; see also Papadakis et al., J Immunol 2004; 172:7002-7007 (“an agonistic anti-DR3 mAb synergize with IL-12/IL-18 to augment IFN-γ production in human peripheral blood T cells and NK cells”), or any other anti-TNFRSF25 agonistic antibody.

In some embodiments, administration of an TNFRSF25 agonistic antibody or antigen binding fragment thereof to a patient in need thereof results in an increase in serum levels of IL-5.

In some embodiments, the present disclosure provides methods for treating or preventing diabetes, prediabetes, and/or glucose intolerance, comprising administering an effective amount of an TNFRSF25 agonistic antibody or antigen binding fragment thereof to a patient in need thereof. In various embodiments, a TNFRSF25 agonistic antibody helps provide a patient with glycemic control, as monitored by, for example, average glucose and/or glycosylated hemoglobin levels. In various embodiments, the anti-diabetic effects of TNFRSF25 agonistic antibody are insulin-independent.

In some embodiments, the present disclosure provides methods for treating or preventing diabetes, prediabetes, and/or glucose intolerance, comprising administering an effective amount of TNFRSF25 agonistic antibody or antigen binding fragment thereof, in combination with an immunosuppressant or anti-inflammatory agent, to a patient in need thereof. In some embodiments, the immunosuppressant or anti-inflammatory agent is an anti-CTLA4 antibody. In some embodiments, the immunosuppressant or anti-inflammatory agent is an IL-1 receptor antagonist. In some embodiments, the IL-1 receptor antagonist is Kineret®. In various embodiments, TNFRSF25 agonistic antibody, in combination with an immunosuppressant or anti-inflammatory agent, helps provide a patient with glycemic control, as monitored by, for example, average glucose and/or glycosylated hemoglobin levels. In various embodiments, the anti-diabetic effects of TNFRSF25 agonistic antibody are insulin-independent.

The patient may be affected by various conditions or diseases. For example, in some embodiments, the patient being treated may be suffering from insulin resistance. As another example, the patient may be diagnosed with one or more of insulin resistance, prediabetes, impaired fasting glucose (IFG), impaired glucose tolerance (IGT), and acanthosis nigricans. In some embodiments, the patient has cardiovascular disease or metabolic disease. As yet another example, the patient can have type 1 diabetes or type 2 diabetes. In some embodiments, the patient has gestational diabetes or steroid-induced diabetes.

The TNFRSF25 agonistic antibody or antigen binding fragment thereof can be administered as a regimen that decreases blood glucose level, stimulates peripheral glucose disposal, and/or inhibits hepatic glucose production. In some embodiments, the patient can have one or more of an average hemoglobin A1c value of more than about 10% and an average glucose of more than about 200 mg/dl (11 mmol/I) at the start of treatment with conventional diabetic therapy. In some embodiments, the TNFRSF25 agonistic antibody or antigen binding fragment thereof is administered to a patient that has one or more of an average hemoglobin Alc value of more than about 11%, or more than about 12%, or more than about 13%, or more than about 14%, or more than about 15% at the start of treatment with conventional diabetic therapy. In some embodiments, the TNFRSF25 agonistic antibody or antigen binding fragment thereof is administered to a patient that has an average glucose of more than about 210 mg/dl, or more than about 220 mg/dl, or more than about 230 mg/dl, or more than about 240 mg/dl, or more than about 250 mg/dl at the start of treatment with conventional diabetic therapy. In various embodiments, the conventional diabetic therapy is any one of those described herein, including, for example, insulin therapy and non-insulin diabetes agent therapy. The non-insulin diabetes agents may include, e.g., metformin, sulfonylureas, glipizide and glimepiride, thiazolidinediones, DPP-4 inhibitors, GLP-1 receptor agonists, and SGLT2 inhibitors.

In some embodiments, the TNFRSF25 agonistic antibody or antigen binding fragment thereof administration is effective for providing average glucose of below about 200 mg/dl (11 mmol/I). In some embodiments, the TNFRSF25 agonistic antibody or antigen binding fragment thereof administration is effective for providing an average glucose of below about 190 mg/dl, or about 180 mg/dl, or about 170 mg/dl, or about 160 mg/dl, or about 150 mg/dl, or about 140 mg/dl, or about 130 mg/dl, or about 120 mg/dl, or about 120 mg/dl, or about 110 mg/dl, or about 100 mg/dl, or about 90 mg/dl, or about 80 mg/dl, or about 70 mg/dl. In some embodiments, TNFRSF25 agonistic antibody or antigen binding fragment thereof administration is effective for providing average glycosylated hemoglobin levels (hemoglobin A1c) values of about 8% or less. For example, in some embodiments, TNFRSF25 agonistic antibody or antigen binding fragment thereof administration is effective for providing an average glycosylated hemoglobin levels (hemoglobin A1c) values of about 8%, or about 7%, or about 6%, or about 5%, or about 4%. In some embodiments, TNFRSF25 agonistic antibody or antigen binding fragment thereof administration is effective for providing average glycosylated hemoglobin levels (hemoglobin A1c) values of less than about 8%, or less than about 7%, or less than about 6%, or less than about 5%, or less than about 4%.

In various embodiments, TNFRSF25 agonistic antibody or antigen binding fragment thereof administration does not cause a patient to experience an increase of insulin upon TNFRSF25 agonistic antibody or antigen binding fragment thereof administration. Accordingly, in some embodiments, TNFRSF25 agonistic antibody's anti-diabetic effects are insulin-independent.

In various embodiments, TNFRSF25 agonistic antibody or antigen binding fragment thereof is administered as an adjuvant therapy. For instance, a diabetic patient may receive treatment with insulin or an insulin analog, or any of the agents listed herein (e.g. sulfonylureas, biguanides, meglitinides, thiazolidinediones, DPP-4 inhibitors, SGLT2 Inhibitors, Alpha-glucosidase inhibitors, and bile acid sequestrants) and TNFRSF25 agonistic antibody or antigen binding fragment thereof is administered to supplement these treatments. For example, in some embodiments, the use of TNFRSF25 agonistic antibody or antigen binding fragment thereof as an adjuvant therapy with a long-acting insulin offsets the high frequency of hypo- and hyperglycemic excursions and modest reduction in HbA1c seen with these agents.

In some embodiments, the patient is undergoing treatment with one or more of insulin or an insulin analog. The insulin analog may be selected from a rapid acting or long acting insulin analog. Non-limiting examples of the rapid acting insulin analog comprise lispro, aspart or glulisine. Non-limiting examples of the long acting insulin analog comprise glargine or detemir.

In embodiments of the present disclosure, administration of TNFRSF25 agonistic antibody or antigen binding fragment thereof does not cause one or more of common side effects of standard diabetes care, such as hypoglycemia or hypokalemia.

In various aspects, the present methods provide for the treatment of diabetes with TNFRSF25 agonistic antibody or antigen binding fragment thereof in specific patient populations in need thereof. For example, in various embodiments, TNFRSF25 agonistic antibody or antigen binding fragment thereof may supplement various agents in a treatment regimen for diabetes, including type 1 or type 2 diabetes, or may supplant various agents in a treatment regimen for diabetes, including type 1 or type 2 diabetes. For example, in some embodiments, TNFRSF25 agonistic antibody or antigen binding fragment thereof is an adjuvant therapy for type 1 or type 2 diabetes.

Type 1 diabetes, once known as juvenile diabetes or insulin-dependent diabetes, is a chronic condition in which the pancreas produces little or no insulin. Treatment is often via intensive insulin regimens, which attempt to mimic the body's normal pattern of insulin secretion, and often involve basal and bolus insulin coverage. For example, one common regimen is the administration of a long-acting insulin (as described herein and including, for example, glargine/detemir) once or twice a day with rapid acting insulin (as described herein and including, for example, aspart, glulisine, lispro) preprandially or postprandially and as needed to correct high blood sugars (as monitored by a glucose meter, for example). Doses administered preprandially or postprandially or as needed to correct high blood sugars may be referred to as bolus administrations. Another common regimen is involves dosing, including continuous dosing, via an insulin pump (or continuous subcutaneous insulin infusion device (CSII)) of, for example a rapid acting insulin (as described herein and including, for example, aspart, glulisine, lispro). In various embodiments, TNFRSF25 agonistic antibody or antigen binding fragment thereof may replace any of the insulins used in various regimens, including instances in which the insulins are not providing effective therapy in the patient. TNFRSF25 agonistic antibody or antigen binding fragment thereof may cause an increase in patient compliance as it may allow for easier self-dosing relative to various forms of insulin, which must be administered as various doses throughout the day, even in the context of an insulin pump, which requires programming.

Further, TNFRSF25 agonistic antibody or antigen binding fragment thereof can offset common frustration of diabetic patient dosing, such as, for example, the dawn phenomenon. Alternatively, TNFRSF25 agonistic antibody or antigen binding fragment thereof may be used adjuvant to any of the type 1 diabetes treatments described herein to, for example, normalize a patient's regimen and avoid blood sugar “dips” (e.g. hypoglycemia, e.g. blood sugar of below about 70 mg/dL) and “spikes” (e.g. hyperglycemia, e.g. blood sugar of below about 200 mg/dL) that afflict many patients. Accordingly, in some embodiments, TNFRSF25 agonistic antibody or antigen binding fragment thereof may treat or prevent symptoms associated with hypoglycemia, including for example, shakiness, anxiety, nervousness, palpitations, tachycardia, pallor, coldness, clamminess, dilated pupils (mydriasis), hunger, borborygmus, nausea, vomiting, abdominal discomfort, headache, abnormal mentation, impaired judgment, nonspecific dysphoria, paresthesia, negativism, irritability, belligerence, combativeness, rage, personality change, emotional lability, fatigue, weakness, apathy, lethargy, daydreaming, sleep, confusion, amnesia, lightheadedness or dizziness, delirium, staring, “glassy” look, blurred vision, double vision, flashes of light in the field of vision, automatism, difficulty speaking, slurred speech, ataxia, incoordination, focal or general motor deficit, paralysis, hemiparesis, paresthesia, headache, stupor, coma, abnormal breathing, generalized or focal seizures, memory loss, and amnesia. Accordingly, in some embodiments, TNFRSF25 agonistic antibody or antigen binding fragment thereof may treat or prevent symptoms associated with hyperglycemia, including for example, polyphagia, polydipsia, polyuria, blurred vision, fatigue, weight loss, poor wound healing, dry mouth, dry or itchy skin, tingling in feet or heels, erectile dysfunction, recurrent infections, external ear infections (e.g. swimmer's ear), cardiac arrhythmia, stupor, coma, and seizures. In various regimens, a type 1 diabetes may receive additional agents to supplement insulin therapy. In some embodiments, TNFRSF25 agonistic antibody or antigen binding fragment thereof are used in this manner. TNFRSF25 agonistic antibody or antigen binding fragment thereof may provide additional therapeutic benefits in patients that are struggling to manage type 1 diabetes with insulin therapy alone. In some embodiments, patients that are struggling to manage type 1 diabetes with insulin therapy alone have poor glycemic control as described herein.

Patients with type 2 diabetes may be instructed to manage their diabetes with healthy eating and exercise. However, certain non-insulin diabetes agents (e.g. selected from metformin (e.g. GLUCOPHAGE, GLUMETZA); sulfonylureas (e.g. glyburide (e.g. DIABETA, GLYNASE), glipizide (e.g. GLUCOTROL) and glimepiride (e.g. AMARYL)); thiazolidinediones (e.g. rosiglitazone (e.g. AVANDIA) and pioglitazone (e.g. ACTOS)); DPP-4 inhibitors (e.g. sitagliptin (e.g. JANUVIA), saxagliptin (e.g. ONGLYZA) and linagliptin (e.g. TRADJENTA)); GLP-1 receptor agonists (e.g. exenatide (e.g. BYETTA) and liraglutide (e.g. VICTOZA)); and SGLT2 inhibitors (e.g. canagliflozin (e.g. NVOKANA) and dapagliflozin (e.g. FARXIGA))) and/or insulin may be used in treatment. For example, certain patients may be able to manage diabetes with diet and exercise alone (e.g. along with glucose monitoring). However, often this is not the case and therapeutic agents are needed. A first line of treatment may be a non-insulin diabetes agent (e.g. selected from metformin (e.g. GLUCOPHAGE, GLUMETZA); sulfonylureas (e.g. glyburide (e.g. DIABETA, GLYNASE), glipizide (e.g. GLUCOTROL) and glimepiride (e.g. AMARYL)); thiazolidinediones (e.g. rosiglitazone (e.g. AVANDIA) and pioglitazone (e.g. ACTOS)); DPP-4 inhibitors (e.g. sitagliptin (e.g. JANUVIA), saxagliptin (e.g. ONGLYZA) and linagliptin (e.g. TRADJENTA)); GLP-1 receptor agonists (e.g. exenatide (e.g. BYETTA) and liraglutide (e.g. VICTOZA)); and SGLT2 inhibitors (e.g. canagliflozin (e.g. NVOKANA) and dapagliflozin (e.g. FARXIGA)). However, some of these agents provide side effects (e.g., in the case of metformin, abdominal or stomach discomfort, cough or hoarseness, decreased appetite, diarrhea, fast or shallow breathing, fever or chills, general feeling of discomfort, lower back or side pain, muscle pain or cramping, painful or difficult urination, and sleepiness) or negative drug interactions (e.g., in the case of metformin, certain imaging and contrast agents). In some embodiments, TNFRSF25 agonistic antibody or antigen binding fragment thereof is used instead of a non-insulin diabetes agent or in combination with one or more non-insulin diabetes agents (e.g., to lower the dose of the non-insulin diabetes agents and increase their therapeutic windows).

In some embodiments, TNFRSF25 agonistic antibody or antigen binding fragment thereof is used to improve an ineffective treatment regimen. In certain embodiments, use of TNFRSF25 agonistic antibody or antigen binding fragment thereof increases patient compliance and increases the likelihood of effective type 2 diabetes management. In certain embodiments, TNFRSF25 agonistic antibody or antigen binding fragment thereof replaces a non-insulin diabetes agent in a patient's treatment regimen in a patient whose diabetes is not well-managed by a non-insulin diabetes agent (e.g. those having uncontrolled, cardiovascular complications and/or blood glucose levels). In some embodiments, a patient whose diabetes is not well-managed by a non-insulin diabetes agent has poor glycemic control as described herein.

In some type 2 diabetes patients, diet and exercise and/or non-insulin diabetes agents are insufficient for treatment of diabetes and treatment with insulin therapy is needed. In various embodiments, TNFRSF25 agonistic antibody or antigen binding fragment thereof may prevent the need to turn to insulin therapy in type 2 diabetes patients or reduce the amount (e.g. frequency of administration) of insulin therapy in type 2 diabetes patients. For example, TNFRSF25 agonistic antibody or antigen binding fragment thereof may be used in certain type 2 diabetes patient populations that are often at risk for needing insulin therapy, including patients afflicted having: acute infections or other serious illnesses, pregnancy, major surgery, congestive heart failure, kidney disease, liver disease, use of other drugs (e.g. prednisone and some psychiatric medications), overeating or excessive weight gain (including obesity), and progressive loss of beta cell function. In some embodiments, TNFRSF25 agonistic antibody or antigen binding fragment thereof may be administered to patients having onset of diabetes prior to age thirty, or a duration over fifteen years to prevent the need for insulin therapy. Further, in some embodiments, TNFRSF25 agonistic antibody or antigen binding fragment thereof is used to treat diabetes in patients at risk for uncontrolled or poorly controlled type 2 diabetes (overweight and/or obese patients, patients with high abdominal fat distribution, inactive patients, patients with a family history. of type 2 diabetes, patients of certain racial groups (e.g., blacks, Hispanics, American Indians and Asian-Americans), older patients (e.g. over the age of about 45), patients previously afflicted with gestational diabetes and/or who have birthed a baby weighing more than about 9 pounds, and patients having polycystic ovary syndrome).

In some embodiments, TNFRSF25 agonistic antibody or antigen binding fragment thereof is used to treat type 2 diabetes patients that have uncontrolled or poorly controlled type 2 diabetes and are facing a nontraumatic lower extremity amputation (LEA). In some embodiments, TNFRSF25 agonistic antibody or antigen binding fragment thereof is used to treat type 2 diabetes patients that have uncontrolled or poorly controlled type 2 diabetes and have some degree of vision loss and/or blindness (by way of non-limiting example, diabetic retinopathy, which may include one or more of non-proliferative diabetic retinopathy (including, for example, treating microanuerysms) and proliferative diabetic retinopathy (including, for example, treating vitreous, clouding vision, detachment of the retina and glaucoma). In some embodiments, the determination of whether a patient is afflicted with or has a high risk for some degree of vision loss and/or blindness comprises diagnostic methods known in the art (e.g. ophthalmoscopy, fluorescein angiography). In some embodiments, TNFRSF25 agonistic antibody or antigen binding fragment thereof is used to treat type 2 diabetes patients that have uncontrolled or poorly controlled type 2 diabetes and have end-stage renal disease (including, for example, end-stage renal disease).

In some aspects, the present disclosure relates a method for treating diabetes and/or glucose intolerance, comprising administering an effective amount of TNFRSF25 agonistic antibody or antigen binding fragment thereof to a patient in need thereof, wherein the patient is not receiving insulin therapy. In some embodiments, the TNFRSF25 agonistic antibody or antigen binding fragment thereof stimulates glucose uptake in the patient. In some embodiments, the glucose uptake is mediated by glucose transporter type 4 (GLUT4). In some embodiments, the glucose uptake is in muscle or fat cells.

The present invention provides, in some aspects, methods of treatment of diabetes and/or glucose intolerance in certain patient populations, including, for example, patients not receiving insulin therapy (e.g., not receiving one or more of basal, preprandial, and postprandial insulin therapy) for various reasons (e.g., ineffectiveness of insulin therapy, allergies, side effects, and lack of compliance) and/or patients that are not receiving non-insulin diabetes agents (e.g., metformin, sulfonylureas, glipizide and glimepiride, thiazolidinediones, DPP-4 inhibitors, GLP-1 receptor agonists, and SGLT2 inhibitors). For example, in some embodiments, the patient is not receiving one or more of basal, preprandial, and postprandial insulin therapy. In some embodiments, the patient is not receiving preprandial or postprandial insulin therapy but is receiving basal insulin therapy. In some embodiments, the patient has not received insulin therapy in up to about 1 hour, or up to about 2 hours, or up to about 3 hours, or up to about 4 hours, or up to about 5 hours, or up to about 6 hours, or up to about 7 hours, or up to about 8 hours, or up to about 12 hours or up to about 16 hours or up to about 20 hours, or up to about 24 hours, up to about 2 days, up to about 3 days, up to about 4 days, up to about 5 days, up to about 6 days, up to about 7 days.

In various embodiments, the patient has experienced one or more instances of lipodystrophy that is caused by injection (e.g., injection of insulin). In some embodiments, the patient is afflicted with or is at risk of having hypokalemia. In some embodiments, the patient is afflicted with or is at risk of having an insulin allergy or allergy to a an agent, such as zinc, commonly used to formulate insulin (e.g., a patient having or who has previously had an immediate hypersensitive reaction upon insulin injection (e.g., injection site swelling, redness and/or itching, local tender subcutaneous nodules which develop about 0.5 to about 6 hours after an insulin injection, inflammation of the lymph glands, or a serum sickness reaction and arthralagia).

In some embodiments, the patient is receiving one or more non-insulin diabetes agents selected from metformin (e.g., GLUCOPHAGE, GLUMETZA); sulfonylureas (e.g., glyburide (e.g., DIABETA, GLYNASE), glipizide (e.g., GLUCOTROL) and glimepiride (e.g., AMARYL)); thiazolidinediones (e.g., rosiglitazone (e.g., AVANDIA) and pioglitazone (e.g., ACTOS)); DPP-4 inhibitors (e.g., sitagliptin (e.g. JANUVIA), saxagliptin (e.g., ONGLYZA) and linagliptin (e.g., TRADJENTA)); GLP-1 receptor agonists (e.g., exenatide (e.g. BYETTA) and liraglutide (e.g., VICTOZA)); and SGLT2 inhibitors (e.g., canagliflozin (e.g. NVOKANA) and dapagliflozin (e.g., FARXIGA)).

In some embodiments, the patient is not receiving one or more non-insulin diabetes agents selected from metformin (e.g., GLUCOPHAGE, GLUMETZA); Sulfonylureas (e.g., glyburide (e.g., DIABETA, GLYNASE), glipizide (e.g., GLUCOTROL) and glimepiride (e.g., AMARYL)); thiazolidinediones (e.g., rosiglitazone (e.g., AVANDIA) and pioglitazone (e.g., ACTOS)); DPP-4 inhibitors (e.g., sitagliptin (e.g., JANUVIA), saxagliptin (e.g., ONGLYZA) and linagliptin (e.g., TRADJENTA)); GLP-1 receptor agonists (e.g., exenatide (e.g., BYETTA) and liraglutide (e.g., VICTOZA)); and SGLT2 inhibitors (e.g., canagliflozin (e.g., NVOKANA) and dapagliflozin (e.g., FARXIGA)).

In some embodiments, the TNFRSF25 agonistic antibody or antigen binding fragment thereof is an adjuvant therapy to an insulin therapy and/or a non-insulin diabetes agent. In some embodiments, the TNFRSF25 agonistic antibody or antigen binding fragment thereof is an insulin replacement therapy, wherein the insulin analog can be selected from a rapid acting insulin analog (e.g., lispro, aspart or glulisine) or a long acting insulin analog (e.g., glargine or detemir). The patient may have type 1 diabetes or type 2 diabetes, gestational diabetes or steroid-induced diabetes. In some embodiments, the treatment of diabetes and/or glucose intolerance in a patients not receiving insulin therapy comprises one or more of a decrease of the blood glucose level, stimulation of peripheral glucose disposal, and inhibition of hepatic glucose production. The TNFRSF25 agonistic antibody or antigen binding fragment thereof administration can be effective for providing glycemic control. The patient may suffer from insulin resistance. In some embodiments, the patient can be diagnosed with one or more of insulin resistance, prediabetes, impaired fasting glucose (IFG), impaired glucose tolerance (IGT), and acanthosis nigricans. The patient may have cardiovascular disease or metabolic disease.

In some embodiments, the TNFRSF25 agonistic antibody or antigen binding fragment thereof is administered as a regimen that decreases blood glucose level, stimulates peripheral glucose disposal, and/or inhibits hepatic glucose production. In some embodiments, the patient has one or more of an average hemoglobin A1c value of more than about 10% and an average glucose of more than about 200 mg/dl (11 mmol/1) at the start of treatment with conventional diabetic therapy, which can be insulin therapy and/or non-insulin diabetes agent therapy. In some embodiments, the TNFRSF25 agonistic antibody or antigen binding fragment thereof administration is effective for providing average glucose of below about 200 mg/dl (11 mmol/1) and/or providing average glycosylated hemoglobin levels (hemoglobin A1c) values of about 8% or less. In some embodiments, the patient does not experience an increase of insulin production upon TNFRSF25 agonistic antibody or antigen binding fragment administration and/or the TNFRSF25 agonistic antibody or antigen binding fragment administration does not cause one or more of hypoglycemia and hypokalemia.

In some aspects, the present disclosure provides a method for increasing a graft survival. The method can comprise administering an effective amount of TNFRSF25 agonistic antibody or antigen binding fragment thereof to a patient in need thereof. In some embodiments, patient is a transplant recipient. The transplant may comprise regulatory T cells (Tregs) from a transplant donor. In some embodiments, the transplant comprises donor hematopoietic cells, donor stem cells, or donor bone marrow cells.

For example, in some embodiments, the method increases a survival of a graft such as a solid organ transplant rejection. In some embodiments, the method reduces the likelihood of solid organ transplant rejection.

The solid organ can be selected from lung, kidney, heart, liver, pancreas, thymus, gastrointestinal tract, cornea, eye, and composite allografts. Composite allograft transplantation can be, in embodiments, intestinal/multivisceral transplantation (which may or may not include liver) and its variants.

In some embodiments, Treg expansion, caused by administration of TNFRSF25 agonistic antibody or antigen binding fragment thereof to a patient in need thereof, is correlated with graft survival, such as with islet transplant survival. In some embodiments, Treg expansion correlated significantly with a fold increase in one or both CD4+FOXP3+ and CD4+CD25+FOXP3+ cells. In embodiments, patient is a diabetes patient. In some embodiments, the patient, which may be afflicted by a diabetes or by another insulin-related condition or disease, is a recipient of a transplant, such as an islet cells transplant, pancreatic transplant, or another transplant.

In some aspects, the present disclosure provides a method for increasing graft survival, comprising administering an effective amount of TNFRSF25 agonistic antibody or antigen binding fragment thereof to a patient in need thereof. In some embodiments, the patient is a transplant recipient. The transplant may comprise islet cells transplant or a solid organ (e.g., pancreas) from a transplant donor. In some embodiments, the patient may be afflicted by a diabetes or by another insulin-related condition or disease. In some embodiments, the increase in the graft survival comprises an increase in at least in at least one month, or at least about 2 months, or at least about 3 months, or at least about 4 months, or at least about 5 months, or at least about 6 months, or at least about 7 months, or at least about 8 months, or at least about 9 months, or at least about 10 months, or at least about 11 months, or at least about 12 months, or at least about a year.

In embodiments, the present methods provide for an increase in a graft survival of greater than about one month, or greater than about 2 months, or greater than about 3 months, or greater than about 4 months, or a greater than about 5 months, or greater than about 6 months, or greater than about 7 months, or greater than about 8 months, or greater than about 9 months, or greater than about 10 months, or greater than about 11 months, or greater than about 12 months, or greater than about year.

In some aspects, the present disclosure provides a method for treating or preventing a graft rejection, comprising administering an effective amount of TNFRSF25 agonistic antibody or antigen binding fragment thereof to a patient in need thereof. In some embodiments, the patient is a transplant recipient. The transplant may comprise islet cells or a solid organ (e.g., pancreas) from a transplant donor. In some embodiments, the patient may be afflicted by a diabetes or by another insulin-related condition or disease.

The present disclosure provides, in some aspects, a method for treating or preventing graft-versus-host disease (GVHD), comprising administering an effective amount of TNFRSF25 agonistic antibody or antigen binding fragment thereof to a patient in need thereof. In some embodiments, the patient is a transplant recipient.

The transplant may comprise islet cells or a solid organ (e.g., pancreas) from a transplant donor. In some embodiments, the transplant comprises donor hematopoietic cells, donor stem cells, or donor bone marrow cells.

In embodiments, the effectiveness of the present methods is assayed by beta cell function. See Forbes et al. American Journal of Transplantation. 2016: 16 (91): pages 2704-2713 (first published: 28 Mar. 2016). In some embodiments, beta cell function is assessed using a single fasting blood sample factoring in the dose of insulin required per day and the patient's body weight. In particular, a BETA-2 score is determined, which is a validated composite score of beta cell function that incorporates the continuous variables glucose, C-peptide, HbA1c and insulin dose and that correlates strongly with other validated measures of graft function. See Forbes et al. American Journal of Transplantation. 2016: 16 (91): pages 2704-2713 (first published: 28 Mar. 2016). The BETA-2 score allows tracking islet engraftment over time, and permits early detection of graft dysfunction. See id. In embodiments, the present methods provide an improvement in BETA-2 score or a comparable measure. It should be appreciated that other approaches can be used additionally or alternatively to access a graft function.

In some embodiments, the treatment or prevention of GVHD using TNFRSF25 agonistic antibody or antigen binding fragment results in reduction of a graft versus host disease. In some embodiments, the graft versus host disease is acute graft-versus-host-disease (aGVHD). In some embodiments, the graft versus host disease is chronic graft-versus-host-disease (cGVHD). In various embodiments, the administration is also to the transplant donor. In some embodiments, the administration to the transplant donor occurs prior to transplant. In some embodiments, the administration to the transplant recipient occurs after the transplant. In some embodiments, administration is to both the transplant donor and transplant recipient.

GVHD is the deterioration of cells or tissues that are transplanted from a donor to a recipient due to the recognition by the immune system of the recipient that the cells or tissues are foreign. Thus, because Class I MHC are on more cells of the body, it is most desirable to transplant cells and tissues from people that have highest matching Class I MHC profiles followed by the highest matching Class MHC profiles. Thus, in most transplant recipients, GVHD is due to activation of the immune system to mismatched Class MHC molecules and other polymorphic proteins (minor histocompatibility antigens).

In some embodiments, the present methods relate to acute and chronic forms of GVHD. The acute or fulminant form of the disease (aGVHD) is normally observed within the first 100 days post-transplant, and is a major challenge to the effectiveness of transplants owing to the associated morbidity and mortality. The chronic form of graft-versus-host-disease (cGVHD) normally occurs after 100 days. The appearance of moderate to severe cases of cGVHD adversely influences long-term survival. After bone marrow transplantation, T cells present in the graft, either as contaminants or intentionally introduced into the host, attack the tissues of the transplant recipient after perceiving host tissues as antigenically foreign. The T cells produce an excess of cytokines, including TNF alpha and interferon-gamma (IFNγ). A wide range of host antigens can initiate graft-versus-host-disease, among them the human leukocyte antigens (HLAs). However, graft-versus-host disease can occur even when HLA-identical siblings are the donors. Classically, acute graft-versus-host-disease is characterized by selective damage to the liver, skin and mucosa, and the gastrointestinal tract. Additional studies show that that other graft-versus-host-disease target organs include the immune system (such as the bone marrow and the thymus) itself, and the lungs in the form of idiopathic pneumonitis. Chronic graft-versus-host-disease also attacks the above organs, but over its long-term course can also cause damage to the connective tissue and exocrine glands.

In some embodiments, the present methods relate to treating or preventing acute GVHD. In embodiments, the present methods treat a patient who has one or more risk factors of acute GVHD, such as HLA “mismatch,” or unrelated donor, older patient age, female donor to male recipient, intensity of the conditioning regimen or total body irradiation during conditioning regimen, and donor lymphocyte infusion. In embodiments, the present methods treat or prevent symptoms of acute GVHD, such as skin rash, gastrointestinal (GI) tract disorders, and liver symptoms.

In some embodiments, the present methods relate to treating or preventing chronic GVHD. In embodiments, the present methods treat a patient who has one or more risk factors of chronic GVHD, such as HLA mismatch or unrelated donor, older patient age, older donor age, female donor for male recipient and number of children the female donor has had, stem cell source, stem cells retrieved from peripheral blood have a higher risk of causing chronic GVHD than stem cells retrieved from bone marrow, stem cells retrieved from cord blood have the lowest risk of causing chronic GVHD, and prior acute GVHD. In embodiments, the present methods treat or prevent symptoms of chronic GVHD, such as symptoms of the eyes, mouth, skin, nails, scalp and body hair, gastrointestinal (GI) tract, lungs, liver, muscles and joints, and genitals and sex organs.

In some embodiments, the present methods relate to GVHD as defined by one of more of the Billingham Criteria: 1) administration of an immunocompetent graft, with viable and functional immune cells; 2) the recipient is immunologically histoincompatible; and 3) the recipient is immunocompromised and therefore cannot destroy or inactivate the transplanted cells.

In some embodiments, the present methods relate to treating or preventing GVHD in a patient/transplant recipient who is undergoing a GVHD treatment. In embodiments, the present methods relate to treating or preventing GVHD in using the present fusion proteins in combination therapies with a GVHD treatment. In embodiments, the present fusion proteins reduce or ameliorate one or more side effects of a GVHD treatment. Illustrative GVHD treatments are immunosuppression agents, such as corticosteroids (such as methylprednisolone or prednisone) and other immunosuppressive drugs. An illustrative GVHD treatment, in some embodiments, is prednisone. Other illustrative GVHD treatments include ibrutinib (e.g. IMBRUVICA), mycophenolate mofetil, mTOR inhibitors, such as sirolimus (rapamycin), everolimus, calcineurin inhibitors, such as tacrolimus or cyclosporine, ciclosporin, monoclonal antibodies such as infliximab (e.g. REMICADE), tocilizumab (e.g. ACTEMRA), alemtuzumab (e.g. CAMPATH), basiliximab (e.g. SIMULECT), daclizumab (e.g. ZINBRYTA), and denileukin diftitox (e.g. ONTAK), antithymocyte globulin (ATG), anti-lymphocyte globulin (ALG), pentostatin (e.g. NIPENT), ruxolitinib (e.g. JAKAFI), and photopheresis.

In some embodiments, the present methods pertain to patients who fail to respond to steroid therapy are labeled “steroid-refractory”. In embodiments, the present methods pertain to patients who fail one or more lines of systemic GVHD therapy.

In embodiments, administration of an TNFRSF25 agonistic antibody or antigen binding fragment thereof to a patient in need thereof results in an increase in serum levels of IL-5.

In various embodiments, the TNFRSF25 agonistic antibody or antigen binding fragment causes a sustained increase in Treg cells in the transplant donor and/or transplant recipient. In some embodiments, the TNFRSF25 agonistic antibody or antigen binding fragment does not cause substantial Treg suppression in the transplant donor and/or transplant recipient. In some embodiments, the TNFRSF25 agonistic antibody or antigen binding fragment thereof does not cause substantial Treg anergy in the transplant donor and/or transplant recipient.

In various embodiments of the present disclosure, the method for treating or preventing GVHD prevents a transplant rejection. For example, in some embodiments, the method prevents a solid organ transplant rejection. In some embodiments, the method reduces the likelihood of solid organ transplant rejection. The solid organ can be selected from lung, kidney, heart, liver, pancreas, thymus, gastrointestinal tract, cornea, eye, and composite allografts. Composite allograft transplantation can be, e.g., intestinal/multivisceral transplantation (which may or may not include liver) and its variants.

The TNFRSF25 agonistic antibody or antigen binding fragment thereof can cause a sustained increase in Treg cells in the solid organ transplant recipient. The sustained increase in Treg cells can comprise a substantially similar level of Treg cells in the solid organ transplant recipient after the first and last administrations of the TNFRSF25 agonistic antibody or antigen binding fragment. For example, if the administration of the TNFRSF25 agonistic antibody or antigen binding fragment occurs 3 times, the increase in Treg cells remains at a substantially similar level in the solid organ transplant recipient after the first and third administrations of the TNFRSF25 agonistic antibody or antigen binding fragment.

In some embodiments, the method prevents a rejection of a transplant that is islet cells transplant. In some embodiments, the method reduces the likelihood of islet cells transplant rejection. Composite allograft transplantation can be, e.g., islet cells transplant rejection and a solid organ transplant such as, pancreas.

The TNFRSF25 agonistic antibody or antigen binding fragment thereof can cause a sustained increase in Treg cells in the islet cells transplant recipient. The sustained increase in Treg cells can comprise a substantially similar level of Treg cells in the islet cells transplant recipient after the first and last administrations of the TNFRSF25 agonistic antibody or antigen binding fragment. For example, if the administration of the TNFRSF25 agonistic antibody or antigen binding fragment occurs 3 times, the increase in Treg cells remains at a substantially similar level in the islet cells transplant recipient after the first and third administrations of the TNFRSF25 agonistic antibody or antigen binding fragment.

In some embodiments in accordance with the present disclosure, the TNFRSF25 agonistic antibody or antigen binding fragment thereof can be administered in various doses. For example, the administration can occur at least 7 times, at least 10 times, at least 14 times, about 3-7 times, about 3-14 times, about 3-21 times, about 3 times, about 7 times, about 10 times, or about 14 times. In some embodiments, the administration occurs daily, which can be, for example, twice daily. In some embodiments, the administration occurs daily for 3-7 days, daily for 7-14 days, daily for 7-21 days, daily for at least 7 days, daily for at least 10 days, or daily for at least 21 days. The administration in any of the above doses can occur before the transplant or concurrently with the transplant. In embodiments in which the transplant is a solid organ transplant, the administration of the TNFRSF25 agonistic antibody or antigen binding fragment thereof in any of the above doses can occur after the solid organ transplant, or before and after the solid organ transplant.

A combination therapy in which the TNFRSF25 agonistic antibody or antigen binding fragment thereof described herein can be administered sequentially or simultaneously with one or more anti-rejection drugs (and in which the anti-rejection drugs can be administered sequentially or simultaneously) can include administration of interleukin-2 (IL-2) (e.g., a low dose of IL-2), which can also be administered sequentially or simultaneously with the TNFRSF25 agonistic antibody or antigen binding fragment thereof and/or with one or more anti-rejection drugs.

Accordingly, in some embodiments, the method of administering TNFRSF25 agonistic antibody or antigen binding fragment thereof comprises comprising administering IL-2. In some embodiments, the IL-2 is a low dose of IL-2. In some embodiments, the low dose of IL-2 is less than 1 million units per square meter per day. In some embodiments, the low dose of IL-2 is an amount in the range of about 30,000 to about 300,000 units per square meter per day. In some embodiments, the low dose of IL-2 is about 300,000 units per square meter per day. In some embodiments, the low dose of IL-2 is about 30,000 units per square meter per day.

In some embodiments, the administration of low dose IL-2 can be sequential with the administration of the TNFRSF25 agonistic antibody or antigen binding fragment thereof. In some embodiments, the administration of low dose IL-2 is concurrent with the administration of the TNFRSF25 agonistic antibody or antigen binding fragment thereof.

In various embodiments, the present invention relates to the generation or modulation of Tregs. A “T regulatory cell” or “Treg cell” refers to a cell that can modulate a T cell response. Treg cells express the transcription factor Foxp3, which is not upregulated upon T cell activation and discriminates Tregs from activated effector cells. Tregs are identified by the cell surface markers CD25, CTLA4, and GITR. Several Treg subsets have been identified that have the ability to inhibit autoimmune, immune, and chronic inflammatory responses and to maintain immune tolerance in tumor-bearing hosts. These subsets include interleukin 10- (IL-10-) secreting T regulatory type 1 (Tr1) cells, transforming growth factor-β- (TGF-β-) secreting T helper type 3 (Th3) cells, and “natural” CD4+/CD25+Tregs (Trn) (Fehervari and Sakaguchi. J. Clin. Invest. 2004, 114:1209-1217; Chen et al. Science. 1994, 265: 1237-1240; Groux et al. Nature. 1997, 389: 737-742).

In some embodiments, the TNFRSF25 agonistic antibody or antigen binding fragment thereof comprises a TNFRSF25 agonistic antibody or antigen binding fragment, such as PTX-35, which is described in PCT/US2017/036817 (WO2017214547), which is incorporated herein by reference in its entirety.

In any of the methods of treatment or prevention of a disease or condition in accordance with the present disclosure, the TNFRSF25 agonistic antibody or antigen binding fragment thereof can comprise (i) a heavy chain variable region comprising heavy chain CDR1, CDR2, and CDR3 sequences, wherein the heavy chain CDR1 sequence is GFTFSNHDLN (SEQ ID NO: 1), the heavy chain CDR2 sequence is YISSASGLISYADAVRG (SEQ ID NO: 2); and the heavy chain CDR3 sequence is DPAYTGLYALDF (SEQ ID NO: 3) or DPPYSGLYALDF (SEQ ID NO: 4); and (ii) a light chain variable region comprising light chain CDR1, CDR2, and CDR3 sequences, wherein the light chain CDR1 sequence is TLSSELSWYTIV (SEQ ID NO: 5), the light chain CDR2 sequence is LKSDGSHSKGD (SEQ ID NO: 6), and the light chain CDR3 sequence is CGAGYTLAGQYGWV (SEQ ID NO: 7). In some embodiments, heavy chain CDR1, CDR2, and CDR3 sequences and/or light chain CDR1, CDR2, and CDR3 sequences each include at least one, or at least 2, or at least 3, or at least 4, or at least 5 mutations such as amino acid substitutions. In some embodiments, heavy chain CDR1, CDR2, and CDR3 sequences and/or light chain CDR1, CDR2, and CDR3 sequences each include more than 5 mutations.

In some embodiments, an TNFRSF25 agonistic antibody includes a set of six CDRs that include no more than two, or no more than three, or no more than four, or no more than five, or no more than six total amino acid substitutions in the set of six CDRs having the amino acid sequences set forth in SEQ ID NOS: 1, 2, 3 or 4, 5, 6, and 7. In some embodiments, an TNFRSF25 agonistic antibody includes a set of six CDRs that include at least one, or at least two, or at least three, or at least four, or at least five total amino acid substitutions in the set of six CDRs having the amino acid sequences set forth in SEQ ID NOS: 1, 2, 3 or 4, 5, 6, and 7. In some embodiments, an TNFRSF25 agonistic antibody includes a set of six CDRs that include one, or two, or three, or four, or five, or more than five total amino acid substitutions in the set of six CDRs having the amino acid sequences set forth in SEQ ID NOS: 1, 2, 3 or 4, 5, 6, and 7.

In some embodiments, the TNFRSF25 agonistic antibody or antigen binding fragment thereof further comprises variable region framework (FW) sequences juxtaposed between the CDRs according to the formula (FW1)-(CDR1)-(FW2)-(CDR2)-(FW3)-(CDR3)-(FW4), wherein the variable region FW sequences in the heavy chain variable region are heavy chain variable region FW sequences, and wherein the variable region FW sequences in the light chain variable region are light chain variable region FW sequences. In some embodiments, the variable region FW sequences are human. In some embodiments, the TNFRSF25 agonistic antibody or antigen binding fragment thereof further comprises human heavy chain and light chain constant regions. The constant regions can be selected from the group consisting of human IgG1, IgG2, IgG3, and IgG4. For instance, in some embodiments, the constant regions are IgG1. In some embodiments, the constant regions are IgG4.

In some embodiments, the TNFRSF25 agonistic antibody or antigen binding fragment thereof comprises a heavy chain variable region having the amino acid sequence EVQLVESGGGLSQPGNSLQLSCEASGFTFSNHDLNWVRQAPGKGLEWVAYISSASGLISYADAVRGRFTISRDN AKNSLFLQMNNLKSEDTAMYYCARDPPYSGLYALDFWGQGTQVTVSS (SEQ ID NO: 8), or an amino acid sequence of at least about 85% to about 99% identity thereto. In some embodiments, the TNFRSF25 agonistic antibody or antigen binding fragment thereof comprises a light chain variable region having the amino acid sequence QPVLTQSPSASASLSGSVKLTCTLSSELSSYTIVWYQQRPDKAPKYVMYLKSDGSHSKGDGIPDRFSGSSSGAH RYLSISNVQSEDDATYFCGAGYTLAGQYGWVFGSGTKVTVL (SEQ ID NO: 9), or an amino acid sequence of at least about 85% to about 99% identity thereto.

In some embodiments, the TNFRSF25 agonistic antibody or antigen binding fragment thereof comprises a heavy chain variable region having the amino acid sequence EVQLVESGGGLSQPGNSLQLSCEASGFTFSNHDLNWVRQAPGKGLEWVAYISSASGLISYADAVRGRFTISRDN AKNSLFLQMNNLKSEDTAMYYCARDPPYSGLYALDFWGQGTQVTVSS (SEQ ID NO: 8), or an amino acid sequence of at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% identity thereto.

In some embodiments, the TNFRSF25 agonistic antibody or antigen binding fragment thereof comprises a light chain variable region having the amino acid sequence QPVLTQSPSASASLSGSVKLTCTLSSELSSYTIVWYQQRPDKAPKYVMYLKSDGSHSKGDGIPDRFSGSSSGAH RYLSISNVQSEDDATYFCGAGYTLAGQYGWVFGSGTKVTVL (SEQ ID NO: 9), or an amino acid sequence of at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% identity thereto.

In some embodiments, a heavy chain variable region has an amino acid sequence of SEQ ID NO: 8, or an antigen binding fragment thereof, but with one to 24 sequence modifications, as well as polypeptides having at least about 80% (e.g., about 85%, about 90%, about 91%, about 92%, about 93%, about 94%, about 95%, about 96%, about 97%, about 98%, or about 99%) amino acid sequence identity to SEQ ID NO: 8, or an antigen binding fragment thereof. In some embodiments, a heavy chain variable region polypeptide can contain 24 or less (e.g., 24, 23, 22, 21, 20, 19, 18, 17, 16, 15, 14, 13, 12, 11, ten, nine, eight, seven, six, five, four, three, two, or one) amino acid substitution as compared to SEQ ID NO: 8, or an antigen binding fragment thereof.

In some embodiment, a light chain variable region has an amino acid sequence of SEQ ID NO: 9, or an antigen binding fragment thereof, but with one to 23 sequence modifications, as well as polypeptides having at least about 80% (e.g., about 85%, about 90%, about 91%, about 92%, about 93%, about 94%, about 95%, about 96%, about 97%, about 98%, or about 99%) amino acid sequence identity to SEQ ID NO: 9, or an antigen binding fragment thereof. In some embodiments, a light chain variable region polypeptide can contain 23 or less (e.g., 23, 22, 21, 20, 19, 18, 17, 16, 15, 14, 13, 12, 11, ten, nine, eight, seven, six, five, four, three, two, or one) amino acid substitutions as compared to SEQ ID NO: 9, or an antigen binding fragment thereof.

In some embodiments, an TNFRSF25 agonistic antibody or antigen binding fragment thereof is PTX-25 antibody or antigen binding fragment thereof. The PTX-25 antibody is described, for example, in PCT/US2015/061082 (WO2016081455), which is incorporated herein by reference in its entirety. In some embodiments, an TNFRSF25 agonistic antibody comprises (i) a heavy chain variable region sequence comprising the amino acid sequence EVQLVESGGGLSQPGNSLQLSCEAS GFTFSNHDLNWVRQAPGKGLEWVAYISSASGLISYADAVRGRFTISRDNAKNSLFLQMNNLKSEDTAMYYCARD PPYSGLYALDFWGQGTQVTVSS (SEQ ID NO: 10) or the amino acid sequence of SEQ ID NO: 10 with no more than 12 total amino acid substitutions (e.g., no more than 12, or no more than 11, or no more than ten, or no more than nine, or no more than eight, or no more than seven, or no more than six, or no more than five, or no more than four, or no more than three, or no more than two total amino acid substitutions); and (ii) a light chain variable region sequence comprising the amino acid sequence QPVLTQSPSASASLSGSVKLTCTLSSEL SSYTIVWYQQRPDKAPKYVMYLKSDGSHSKGDGIPDRFSGSSSGAHRYLSISNVQSEDDATYFCGAGYTLAGQ YGWVFGSGTKVTVL (SEQ ID NO:11) or the amino acid sequence of SEQ ID NO: 11 with no more than 11 total amino acid substitutions (e.g., no more than 11, or no more than ten, or no more than nine, or no more than eight, or no more than seven, or no more than six, or no more than five, or no more than four, or no more than three, or no more than two total amino acid substitutions, or no more than one total amino acid substitution). An amino acid substitution refers to the replacement of one amino acid residue with another amino acid in a peptide sequence.

In some embodiments, the TNFRSF25 agonistic antibody or antigen binding fragment thereof comprises a heavy chain variable region having the amino acid sequence EVQLVESGGGLSQPGNSLQLSCEAS GFTFSNHDLNWVRQAPGKGLEWVAYISSASGLISYADAVRGRFTISRDNAKNSLFLQMNNLKSEDTAMYYCARD PPYSGLYALDFWGQGTQVTVSS (SEQ ID NO: 10) or an amino acid sequence of at least about 85 to about 99% (e.g., an amino acid sequence of at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) identity thereto.

In some embodiments, the TNFRSF25 agonistic antibody or antigen binding fragment thereof comprises a light chain variable region having the amino acid sequence QPVLTQSPSASASLSGSVKLTCTLSSELSSYTIVWYQQRPDKAPKYVMYLKSDGSHSKGDGIPDRFSGSSSGAH RYLSISNVQSEDDATYFCGAGYTLAGQYGWVFGSGTKVTVL (SEQ ID NO: 11), or an amino acid sequence of at least about 85 to about 99% (e.g., an amino acid sequence of at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) identity thereto.

In some embodiments, an TNFRSF25 agonistic antibody or antigen binding fragment thereof comprises an isolated heavy chain variable region polypeptide that binds specifically to TNFRSF25, where the polypeptide includes heavy chain CDR1, CDR2, and CDR3 sequences, where the CDR1 sequence is GFTFSNHDLN (SEQ ID NO: 12), the CDR2 sequence is YISSASGLISYADAVRG (SEQ ID NO: 13); and (c) the CDR3 sequence is DPPYSGLYALDF (SEQ ID NO: 14). The isolated heavy chain variable region polypeptide can further include variable region heavy chain framework (FW) sequences juxtaposed between the heavy chain CDRs according to the formula (FW1)-(CDR1)-(FW2)-(CDR2)-(FW3)-(CDR3)-(FW4). The heavy chain framework sequences can be human. In some embodiments, the isolated heavy chain variable region polypeptide can be combination with a light chain variable region polypeptide. In some embodiments, the light chain variable region polypeptide comprises light chain CDR1, CDR2, and CDR3 sequences, wherein the CDR1 sequence is TLSSELSSYTIV (SEQ ID NO: 15), the CDR2 sequence is LKSDGSHSKGD (SEQ ID NO: 16), and the CDR3 sequence is GAGYTLAGQYGWV (SEQ ID NO: 17). The TNFRSF25 agonistic antibody can be a humanized monoclonal antibody that specifically binds to TNFRSF25.

In some embodiments, an TNFRSF25 agonistic antibody includes a set of six CDRs that include no more than two, or no more than three, or no more than four, or no more than five, or no more than six total amino acid substitutions in the set of six CDRs having the amino acid sequences set forth in SEQ ID NOS: 12, 13, 14, 15, 16, and 17. In some embodiments, an TNFRSF25 agonistic antibody includes a set of six CDRs that include at least one, or at least two, or at least three, or at least four, or at least five, or at least 6 total amino acid substitutions in the set of six CDRs having the amino acid sequences set forth in SEQ ID NOS: 12, 13, 14, 15, 16, and 17. In some embodiments, an TNFRSF25 agonistic antibody includes a set of six CDRs that include one, or two, or three, or four, or five, or more than five total amino acid substitutions in the set of six CDRs having the amino acid sequences set forth in SEQ ID NOS: 12, 13, 14, 15, 16, and 17.

In embodiments, the TNFRSF25 agonistic antibody or antigen binding fragment thereof comprises a heavy chain comprising an amino acid of SEQ ID NO: 18, 19, 20, or 21, and a light chain comprising an amino acid of SEQ ID NO: 22 or 23, as follows:

(SEQ ID NO: 18)
EVQLVESGGGLVQPGGSLRLSCEASGFTFSNHD
LNWVRQAPGKGLEWAYISSASGLISYADAVRGR
FTISRDNAKNSLYLQMNSLRAEDTAVYYCARDP
PYSGLYALDFWGQGTQVTVSS,
(SEQ ID NO: 19)
EVQLVESGGGLVQPGGSLRLSCAASGFTFSNHD
LNWVRQAPGKGLEWAYISSASGLISYADAVRGR
FTISRDNAKNSLYLQMNSLRAEDTAVYYCARDP
PYSGLYALDFWGQGTQVTVSS,
(SEQ ID NO: 20)
EVQLVESGGGLVQPGGSLRLSCEASGFTFSNHD
LNWVRQAPGKGLEWSYISSASGLISYADAVRGR
FTISRDNAKNSLYLQMNSLRAEDTAVYYCARDP
PYSGLYALDFWGQGTQVTVSS,
(SEQ ID NO: 21)
EVQLVESGGGLVQPGGSLRLSCAASGFTFSNHD
LNWVRQAPGKGLEWSYISSASGLISYADAVRGR
FTISRDNAKNSLYLQMNSLRAEDTAVYYCARDP
PYSGLYALDFWGQGTQVTVSS,
(SEQ ID NO: 22)
QPVLTQSSSASASLGSSVKLTCTLSSELSSYTI
VWHQQQPGKAPRYLMYLKSDGSHSKGDGVPDRF
SGSSSGADRYLTISNLQSEDEADYYCGAGYTLA
GQYGWFGSGTKVTVL,
(SEQ ID NO: 23)
QLVLTQSPSASASLGASVKLTCTLSSELSSYTI
VWHQQQPEKGPRYLMYLKSDGSHSKGDGIPDRF
SGSSSGAERYLTISSLQSEDEADYYCGAGYTLA
GQYGWVFGSGTKVTVL.

In embodiments, an TNFRSF25 agonistic antibody or antigen binding fragment thereof is any of the antibodies or antibody fragments (or combinations thereof) described in PCT/US2015/061082 (WO2016081455).

In embodiments, variable region light chain framework (FW) sequences can be juxtaposed between the light chain CDRs according to the formula (FW1)-(CDR1)-(FW2)-(CDR2)-(FW3)-(CDR3)-(FW4). The light chain framework sequences can be human. The antibody or antigen binding fragment can further include a human constant region (e.g., a constant region selected from the group consisting of human IgG1, IgG2, IgG3, and IgG4), or a murine constant region (e.g., a constant region selected from the group consisting of murine IgG1, IgG2A, IgG2B, and IgG3).

In some embodiments, an TNFRSF25 agonistic antibody or antigen binding fragment thereof is as described in PCT/US2010/044218 (WO2011017303), which is incorporated herein by reference in its entirety.

Various embodiments of the present disclosure make use of TNFRSF25 agonistic antibodies or antigen binding fragment thereof. In various embodiments, the antibody is an antibody (e.g., human, hamster, feline, mouse, cartilaginous fish, or camelid antibodies), and any derivative or conjugate thereof, that specifically binds to TNFRSF25. Non-limiting examples of antibodies include monoclonal antibodies, polyclonal antibodies, humanized antibodies, multi-specific antibodies (e.g., bi-specific antibodies), single-chain antibodies (e.g., single-domain antibodies, camelid antibodies, and cartilaginous fish antibodies), chimeric antibodies, feline antibodies, and felinized antibodies. Monoclonal antibodies are homogeneous populations of antibodies to a particular epitope of an antigen. Polyclonal antibodies are heterogeneous populations of antibody molecules that are contained in the sera of the immunized animals.

An isolated polypeptide can yield a single major band on a non-reducing polyacrylamide gel. An isolated polypeptide can be at least about 75% pure (e.g., at least 80%, 85%, 90%, 95%, 97%, 98%, 99%, or 100% pure). Isolated polypeptides can be obtained by, for example, extraction from a natural source, by chemical synthesis, or by recombinant production in a host cell or transgenic plant, and can be purified using, for example, affinity chromatography, immunoprecipitation, size exclusion chromatography, and ion exchange chromatography. The extent of purification can be measured using any appropriate method, including, without limitation, column chromatography, polyacrylamide gel electrophoresis, or high-performance liquid chromatography.

In embodiments, an antigen binding fragment that specifically binds to TNFRSF25 is provided. Such antigen binding fragment, in embodiments, is any portion of a full-length antibody that contains at least one variable domain (e.g., a variable domain of a mammalian (e.g., feline, human, hamster, or mouse) heavy or light chain immunoglobulin, a camelid variable antigen binding domain (VHH), or a cartilaginous fish immunoglobulin new antigen receptor (Ig-NAR) domain) that is capable of specifically binding to an antigen. Non-limiting examples of antibody fragments include Fab, Fab′, F(ab′)2, and Fv fragments, diabodies, linear antibodies, and multi-specific antibodies formed from antibody fragments. Additional antibody fragments containing at least one camelid VHH domain or at least one cartilaginous fish Ig-NAR domain include mini-bodies, micro-antibodies, subnano-antibodies, and nano-antibodies, and any of the other forms of antibodies described, for example, in U.S. Publication No. 2010/0092470.

An antibody can be of the IgA-, IgD-, IgE, IgG- or IgM-type, including IgG- or IgM-types such as, without limitation, IgG1-, IgG2-, IgG3-, IgG4-, IgM1- and IgM2-types. For example, in some cases, the antibody is of the IgG1-, IgG2- or IgG4-type.

In some embodiments, antibodies as provided herein can be fully human or humanized antibodies. In embodiments, the human antibody is an antibody that is encoded by a nucleic acid (e.g., a rearranged human immunoglobulin heavy or light chain locus) present in the genome of a human. In some embodiments, a human antibody can be produced in a human cell culture (e.g., feline hybridoma cells). In some embodiments, a human antibody can be produced in a non-human cell (e.g., a mouse or hamster cell line). In some embodiments, a human antibody can be produced in a bacterial or yeast cell.

Human antibodies can avoid certain problems associated with xenogeneic antibodies, such as antibodies that possess murine or rat variable and/or constant regions. For example, because the effector portion is human, it can interact better with other parts of the human immune system, e.g., to destroy target cells more efficiently by complement-dependent cytotoxicity or antibody-dependent cellular cytotoxicity. In addition, the human immune system should not recognize the antibody as foreign. Further, half-life in human circulation will be similar to naturally occurring human antibodies, allowing smaller and less frequent doses to be given. Methods for preparing human antibodies are known in the art.

In embodiments, the antibody is a humanized antibody, e.g., an antibody that contains minimal sequence derived from non-human (e.g., mouse, hamster, rat, rabbit, or goat) immunoglobulin. Humanized antibodies generally are chimeric or mutant monoclonal antibodies from mouse, rat, hamster, rabbit or other species, bearing human constant and/or variable region domains or specific changes. In non-limiting examples, humanized antibodies are human antibodies (recipient antibody) in which hypervariable region (HVR) residues of the recipient antibody are replaced by HVR residues from a non-human species (donor) antibody, such as a mouse, rat, rabbit, or goat antibody having the desired specificity, affinity, and capacity. In some embodiments, Fv framework residues of the human immunoglobulin can be replaced by corresponding non-human residues. In some embodiments, humanized antibodies can contain residues that are not found in the recipient antibody or in the donor antibody. Such modifications can be made to refine antibody performance, for example.

In some embodiments, a humanized antibody can contain substantially all of at least one, and typically two, variable domains, in which all or substantially all of the hypervariable loops (CDRs) correspond to those of a non-human immunoglobulin, while all or substantially all of the framework regions are those of a human immunoglobulin sequence. A humanized antibody also can contain at least a portion of an immunoglobulin constant (Fc) region, typically that of a human immunoglobulin.

In some embodiments, a humanized antibody or antigen binding fragment as provided herein can have reduced or minimal effector function (e.g., as compared to corresponding non-humanized antibody), such that it does not stimulate effector cell action to the same extent that a corresponding non-humanized antibody would.

Techniques for generating humanized antibodies are well known to those of skill in the art. In some embodiments, controlled rearrangement of antibody domains joined through protein disulfide bonds to form new, artificial protein molecules or “chimeric” antibodies can be utilized (Konieczny et al., Haematologia (Budap.) 14:95, 1981). Recombinant DNA technology can be used to construct gene fusions between DNA sequences encoding mouse antibody variable light and heavy chain domains and human antibody light and heavy chain constant domains (Morrison et al., Proc Natl Acad Sci USA 81:6851, 1984). For example, DNA sequences encoding antigen binding portions or CDRs of murine monoclonal antibodies can be grafted by molecular means into DNA sequences encoding frameworks of human antibody heavy and light chains (Jones et al., Nature 321:522, 1986; and Riechmann et al., Nature 332:323, 1988). Expressed recombinant products are called “reshaped” or humanized antibodies, and contain the framework of a human antibody light or heavy chain and antigen recognition portions, CDRs, of a murine monoclonal antibody.

Other methods for designing heavy and light chains and for producing humanized antibodies are described in, for example, U.S. Pat. Nos. 5,530,101; 5,565,332; 5,585,089; 5,639,641; 5,693,761; 5,693,762; and 5,733,743. Yet additional methods for humanizing antibodies are described in U.S. Pat. Nos. 4,816,567; 4,935,496; 5,502,167; 5,558,864; 5,693,493; 5,698,417; 5,705,154; 5,750,078; and 5,770,403, for example.

In embodiments, the antibody is a single-chain antibody, e.g. a single polypeptide that contains at least one variable binding domain (e.g., a variable domain of a mammalian heavy or light chain immunoglobulin, a camelid VHH, or a cartilaginous fish (e.g., shark) Ig-NAR domain) that is capable of specifically binding to an antigen. Non-limiting examples of single-chain antibodies include single-domain antibodies.

In embodiments, the antibody is a single-domain antibody, e.g. a polypeptide that contains one camelid VHH or at least one cartilaginous fish Ig-NAR domain that is capable of specifically binding to an antigen. Non-limiting examples of single-domain antibodies are described, for example, in U.S. Publication No. 2010/0092470.

In embodiments, the antibody specifically binds to a particular antigen, e.g., TNFRSF25, when it binds to that antigen in a sample, and does not recognize and bind, or recognizes and binds to a lesser extent, other molecules in the sample. In some embodiments, an antibody or an antigen binding fragment thereof can selectively bind to an epitope with an affinity (Kd) equal to or less than, for example, about 1×10⁻⁶ M (e.g., equal to or less than about 1×10⁻⁹ M, equal to or less than about 1×10⁻¹⁰ M, equal to or less than about 1×10⁻¹¹ M, or equal to or less than about 1×10⁻¹² M) in phosphate buffered saline. The ability of an antibody or antigen binding fragment to specifically bind a protein epitope can be determined using any of the methods known in the art or those methods described herein (e.g., by Biacore/Surface Plasmon Resonance). This can include, for example, binding to TNFRSF25 on live cells as a method to stimulate caspase activation in live transformed cells, binding to an immobilized target substrate including human TNFRSF25 fusion proteins as detected using an ELISA method, binding to TNFRSF25 on live cells as detected by flow cytometry, or binding to an immobilized substrate by surface plasmon resonance (including ProteOn).

Antibodies having specific binding affinity for TNFRSF25 can be produced using standard methods. For example, a TNFRSF25 polypeptide can be recombinantly produced, purified from a biological sample (e.g., a heterologous expression system), or chemically synthesized, and used to immunize host animals, including rabbits, chickens, mice, guinea pigs, or rats. Various adjuvants that can be used to increase the immunological response depend on the host species and include Freund's adjuvant (complete and incomplete), mineral gels such as aluminum hydroxide, surface active substances such as lysolecithin, pluronic polyols, polyanions, peptides, oil emulsions, keyhole limpet hemocyanin and dinitrophenol. Monoclonal antibodies can be prepared using a TNFRSF25 polypeptide and standard hybridoma technology. In particular, monoclonal antibodies can be obtained by any technique that provides for the production of antibody molecules by continuous cell lines in culture such as described by Kohler et al. (Nature 256:495, 1975), the human B-cell hybridoma technique of Kosbor et al. (Immunology Today, 4:72, 1983) or Cote et al. (Proc. Natl. Acad. Sci. USA, 80:2026, 1983), and the EBV-hybridoma technique described by Cole et al. (Monoclonal Antibodies and Cancer Therapy, Alan R. Liss, Inc., pp. 77-96, 1983). Such antibodies can be of any immunoglobulin class including IgG, IgM, IgE, IgA, IgD, and any subclass thereof. The hybridoma producing the monoclonal antibodies can be cultivated in vitro and in vivo.

In some embodiments, amino acid substitutions can be made by selecting conservative substitutions that do not differ significantly in their effect on maintaining (a) the structure of the peptide backbone in the area of the substitution, (b) the charge or hydrophobicity of the molecule at the target site, or (c) the bulk of the side chain. For example, naturally occurring residues can be divided into groups based on side-chain properties: (1) hydrophobic amino acids (norleucine, methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Non-limiting examples of conservative substitutions include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenylalanine, glycine for proline, threonine for serine, serine for threonine, tyrosine for tryptophan, phenylalanine for tyrosine, and/or leucine for valine. In some embodiments, an amino acid substitution can be non-conservative, such that a member of one of the amino acid classes described above is exchanged for a member of another class.

Pharmaceutical Compositions

In addition, the disclosure also provides pharmaceutical compositions for the present methods of treating diabetes and related disorders, and GVHD (acute or chronic), which include an antibody or antigen binding fragment, as described herein, in combination with a pharmaceutically acceptable carrier. A “pharmaceutically acceptable carrier” (also referred to as an “excipient” or a “carrier”) is a pharmaceutically acceptable solvent, suspending agent, stabilizing agent, or any other pharmacologically inert vehicle for delivering one or more therapeutic compounds to a subject (e.g., a mammal, such as a human, non-human primate, dog, cat, sheep, pig, horse, cow, mouse, rat, or rabbit), which is nontoxic to the cell or subject being exposed thereto at the dosages and concentrations employed. Pharmaceutically acceptable carriers can be liquid or solid, and can be selected with the planned manner of administration in mind so as to provide for the desired bulk, consistency, and other pertinent transport and chemical properties, when combined with one or more of therapeutic compounds and any other components of a given pharmaceutical composition. Typical pharmaceutically acceptable carriers that do not deleteriously react with amino acids include, by way of example and not limitation: water, saline solution, binding agents (e.g., polyvinylpyrrolidone or hydroxypropyl methylcellulose), fillers (e.g., lactose and other sugars, gelatin, or calcium sulfate), lubricants (e.g., starch, polyethylene glycol, or sodium acetate), disintegrates (e.g., starch or sodium starch glycolate), and wetting agents (e.g., sodium lauryl sulfate). Pharmaceutically acceptable carriers also include aqueous pH buffered solutions or liposomes (small vesicles composed of various types of lipids, phospholipids and/or surfactants which are useful for delivery of a drug to a mammal). Further examples of pharmaceutically acceptable carriers include buffers such as phosphate, citrate, and other organic acids, antioxidants such as ascorbic acid, low molecular weight (less than about 10 residues) polypeptides, proteins such as serum albumin, gelatin, or immunoglobulins, hydrophilic polymers such as polyvinylpyrrolidone, amino acids such as glycine, glutamine, asparagine, arginine or lysine, monosaccharides, disaccharides, and other carbohydrates including glucose, mannose or dextrins, chelating agents such as EDTA, sugar alcohols such as mannitol or sorbitol, salt-forming counterions such as sodium, and/or nonionic surfactants such as TWEEN™, polyethylene glycol (PEG), and PLURONICS™.

Pharmaceutical compositions can be formulated by mixing one or more active agents with one or more physiologically acceptable carriers, diluents, and/or adjuvants, and optionally other agents that are usually incorporated into formulations to provide improved transfer, delivery, tolerance, and the like. A pharmaceutical composition can be formulated, e.g., in lyophilized formulations, aqueous solutions, dispersions, or solid preparations, such as tablets, dragées or capsules. A multitude of appropriate formulations can be found in the formulary known to all pharmaceutical chemists: Remington's Pharmaceutical Sciences (18t^{h} ed, Mack Publishing Company, Easton, Pa. (1990)), particularly Chapter 87 by Block, Lawrence, therein. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies as described herein, provided that the active agent in the formulation is not inactivated by the formulation and the formulation is physiologically compatible and tolerable with the route of administration. See, also, Baldrick, Regul Toxicol Pharmacol 32:210-218, 2000; Wang, Int J Pharm 203:1-60, 2000; Charman, J Pharm Sci 89:967-978, 2000; and Powell et al. PDA J Pharm Sci Technol 52:238-311, 1998), and the citations therein for additional information related to formulations, excipients and carriers well known to pharmaceutical chemists.

Pharmaceutical compositions include, without limitation, solutions, emulsions, aqueous suspensions, and liposome-containing formulations. These compositions can be generated from a variety of components that include, for example, preformed liquids, self-emulsifying solids and self-emulsifying semisolids. Emulsions are often biphasic systems comprising of two immiscible liquid phases intimately mixed and dispersed with each other; in general, emulsions are either of the water-in-oil (w/o) or oil-in-water (o/w) variety. Emulsion formulations have been widely used for oral delivery of therapeutics due to their ease of formulation and efficacy of solubilization, absorption, and bioavailability.

Compositions and formulations can contain sterile aqueous solutions, which also can contain buffers, diluents and other suitable additives (e.g., penetration enhancers, carrier compounds and other pharmaceutically acceptable carriers). Compositions additionally can contain other adjunct components conventionally found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions provided herein, such as dyes, flavoring agents, preservatives, antioxidants, opacifiers, thickening agents and stabilizers. Furthermore, the composition can be mixed with auxiliary agents, e.g., lubricants, preservatives, stabilizers, wetting agents, emulsifiers, salts for influencing osmotic pressure, buffers, colorings, flavorings, and aromatic substances. When added, however, such materials should not unduly interfere with the biological activities of the polypeptide components within the compositions provided herein. The formulations can be sterilized if desired.

In some embodiments, a composition containing an antibody or antigen binding fragment as used herein can be in the form of a solution or powder with or without a diluent to make an injectable suspension. The composition may contain additional ingredients including, without limitation, pharmaceutically acceptable vehicles, such as saline, water, lactic acid, mannitol, or combinations thereof, for example.

In one aspect, the disclosure provides a method of making an anti-TNFRSF25 antibody or antigen binding fragment thereof, comprising: a) providing the host cell as described herein; b) culturing said host cell under conditions wherein said antibody is expressed; and c) recovering said antibody from the host cell.

Any appropriate method can be used to administer an antibody or antigen binding fragment as described herein to a mammal. Administration can be, for example, parenteral (e.g., by subcutaneous, intrathecal, intraventricular, intramuscular, or intraperitoneal injection, or by intravenous drip). Administration can be rapid (e.g., by injection) or can occur over a period of time (e.g., by slow infusion or administration of slow release formulations). In some embodiments, administration can be topical (e.g., transdermal, sublingual, ophthalmic, or intranasal), pulmonary (e.g., by inhalation or insufflation of powders or aerosols), or oral. In addition, a composition containing an antibody or antigen binding fragment as described herein can be administered prior to, after, or in lieu of surgical resection of a tumor.

A composition containing an anti-TNFRSF25 antibody or antigen binding fragment can be administered to a mammal in any appropriate amount, at any appropriate frequency, and for any appropriate duration effective to achieve a desired outcome. For example, an anti-TNFRSF25 antibody or antigen binding fragment can be administered to a subject in an amount effective to stimulate proliferation of T cells in vitro or in vivo (e.g., human, murine, hamster, or macaque T cells, including CD8+ T cells and/or CD4+FoxP3+ regulatory T cells), to stimulate apoptosis of tumor cells that express TNFRSF25, to reduce tumor size, or to increase progression-free survival of a cancer patient. In some embodiments, an anti-TNFRSF25 antibody or antigen binding fragment can be administered at a dosage of about 0.1 mg/kg to about 10 mg/kg (e.g., about 0.1 mg/kg to about 1 mg/kg, about 1 mg/kg to about 5 mg/kg, or about 5 mg/kg to about 10 mg/kg), and can be administered once every one to three weeks (e.g., every week, every 10 days, every two weeks, or every three weeks).

In some cases, a composition containing an anti-TNFRSF25 antibody or antigen binding fragment as described herein can be administered to a subject in an amount effective to increase proliferation of T cells (e.g., by at least about 10 percent, about 20 percent, about 25 percent, about 50 percent, about 60 percent, about 70 percent, about 75 percent, about 80 percent, about 90 percent, about 100 percent, or more than 100 percent), as compared to the “baseline” level of T cell proliferation in the subject prior to administration of the composition, or as compared to the level of T cell proliferation in a control subject or population of subjects to whom the composition was not administered. The T cells can be, for example, CD4+FoxP3+ T cells, regulatory T cells. Any suitable method can be used to determine whether or not the level of T cell proliferation is increased in the subject. Such methods can include, without limitation, flow cytometry analysis of antigen specific T cells (e.g., flow cytometry analysis of the proportion of antigen specific CD4+FoxP3+ T cells as a fraction of the total CD4+ T cell pool), analysis of cell proliferation markers (e.g., expression of Ki67) in CD⁴⁺ T cells, increased counts of CD4+ T cells, or increased proportions of individual TCR sequences of a particular clone of CD⁴⁺ T cells.

Any aspect or embodiment described herein can be combined with any other aspect or embodiment as disclosed herein.

In order that the invention disclosed herein may be more efficiently understood, examples are provided below. It should be understood that these examples are for illustrative purposes only and are not to be construed as limiting the invention in any manner.

### Examples

### Example 1: In Vivo Expansion Of Regulatory T-cells in Islet Transplantation

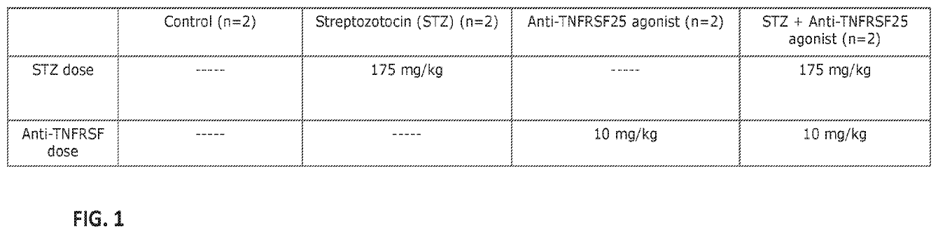

FIGS. 1-3 illustrate in vivo expansion of regulatory T cells in islet transplantation by stimulation of TNFRSF25. An objective of this study was to determine whether administration of anti-TNFRSF25 agonist (mPTX-35), can induce the proliferation of regulatory T cells in vivo in a murine model.

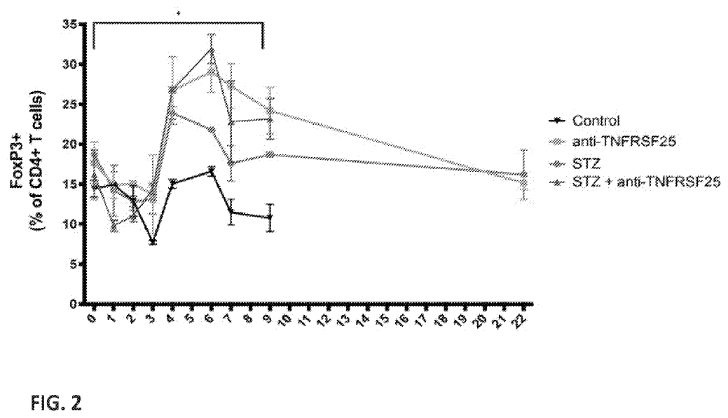

FIG. 1 illustrates schematically the design of the present study. As shown, mice (n=2 in each group) were injected with saline (control), streptozotocin (STZ), mouse anti-TNFRSF25 agonist (“Anti-TNFRSF25 agonist”), and combined STZ+anti-TNFRSF25 treatment (“STZ+Anti-TNFRSF25 agonist”). STZ dose was 175 mg/kg, and anti-TNFRSF25 agonist dose was 10 mg/kg. Blood flow cytometry was performed to assess the Treg response. Statistically significant differences were observed between groups (*One-way ANOVA of repeated measurements, p=0.0004). FIG. 2 illustrates kinetics of CD4+/FoxP3+(T regs) through time—days 0 to 22.

Streptozotocin (STZ, 2-deoxy-2-(3-(methyl-3-nitrosoureido)-D-glucopyranose)) is a naturally occurring alkylating antineoplastic agent that selectively destroys insulin-producing beta cells of the pancreas in mammals. STZ is used to induce experimental diabetes in rodents. As shown in FIG. 2, STZ does not prevent anti-TNFRSF25 agonist from performing its desired effect of expanding of T regs.

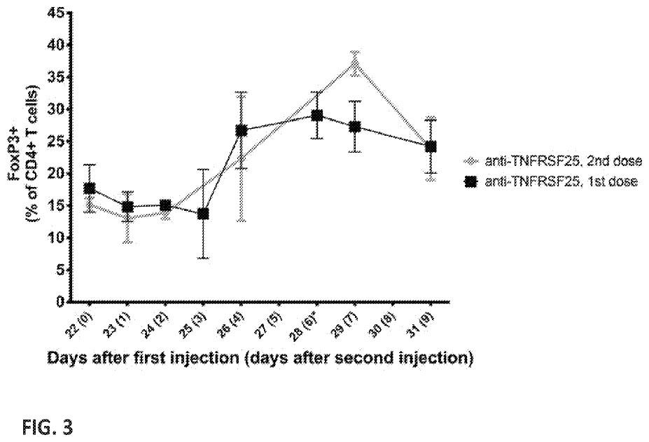

In the study of FIG. 1, on day 22, the mice (n=2) were injected the second dose of anti-TNFRSF25 agonist, and CD4+/FoxP3+ regulatory T-cells were monitored through time. FIG. 3 illustrates kinetics of CD4+/FoxP3+ regulatory T-cells through time—days 22 through 31 after the first injection of anti-TNFRSF25 agonist, and days 0 to 9 after the second injection of anti-TNFRSF25 agonist. Blood flow cytometry was performed to assess the T-reg response. As shown in FIG. 3, a peak at day 7 post-injection (+29 of the first injection) was observed. The results shown in FIGS. 2 and 3 demonstrate that in vivo, the anti-TNFRSF25 agonist induces T-reg proliferation and that streptozotocin does not prevent the anti-TNFRSF25 agonist from inducing T-reg proliferation. As shown in FIG. 3, no statistically significant difference was observed between the results for the first and second doses of anti-TNFRSF25 agonist. The second anti-TNFRSF25 agonist dose had the same effect on the T-reg population as the first dose. This suggests, without wishing to be bound by the theory, that multiple dosing scheme of anti-TNFRSF25 agonist can be used. These findings demonstrate that mPTX-35 can mitigate immune rejection and can promote immunological tolerance associated with islet transplantation, which can prevent the use of chronic immunosuppression in islet transplantation.

### Example 2: TNF Receptor Superfamily Member 25 (TNFRSF25) Antibody in a Murine Model of Islet Transplantation

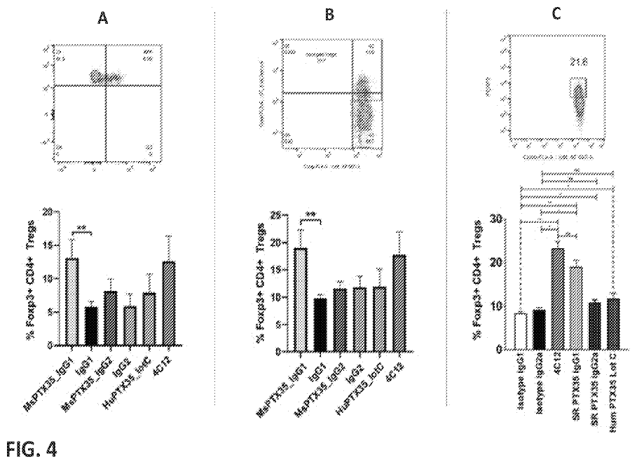

FIGS. 4-8 demonstrate that mouse PTX-35 IgG1 surrogate is effective in expanding FoxP3+CD4+ T regs, which are a non-redundant T cell subset required to maintain peripheral immune tolerance. FIG. 4 shows results for three groups of mice (Panels A, B, and C) on day 6. In the experiment of FIG. 4, FoxP3+CD4+ T cells expansion is shown in the following analyzed groups (in panels A and B): mouse PTX-35 IgG1 (“msPTX-35 IgG1”), IgG1, msPTX-35 IgG2, IgG2, human PTX-35 lot C (“HuPTX35_lotC”), and 4C12. In panel C, the analyzed groups were the same in the panels A and B, but somewhat different notations are used (and the order of the presentation is different): IgG1 (“Isotype IgG1”), IgG2 (“Isotype IgG2a”), 4C12, mouse PTX-35 IgG1 (“SR (surrogate) PTX-35 IgG2a”), and human PTX-35 lot C (“Hum PTX-35 Lot C”). In each of the panels A to C, the top graph shows flow cytometry data and the bottom bar chart illustrates percentage of FoxP3+CD4+ T regs. As shown in FIG. 4, the mouse PTX-35 IgG1 results in the expansion of FoxP3+CD4+ T regs similar to the effect of 4C12 or more prominently than 4C12 (see panels A and B).

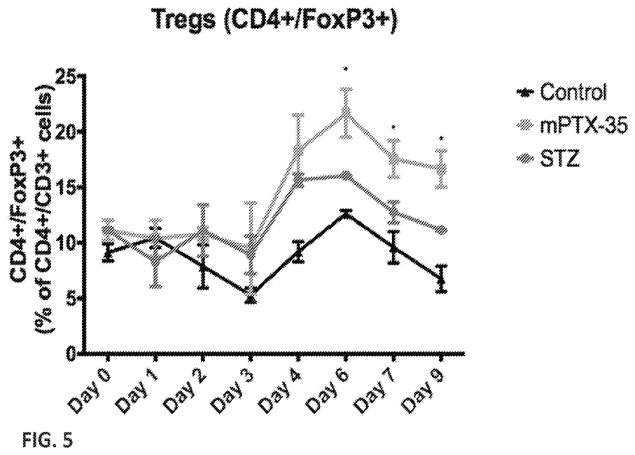

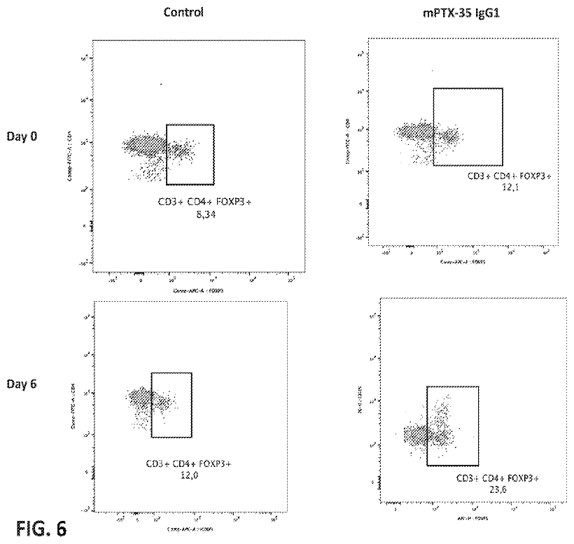

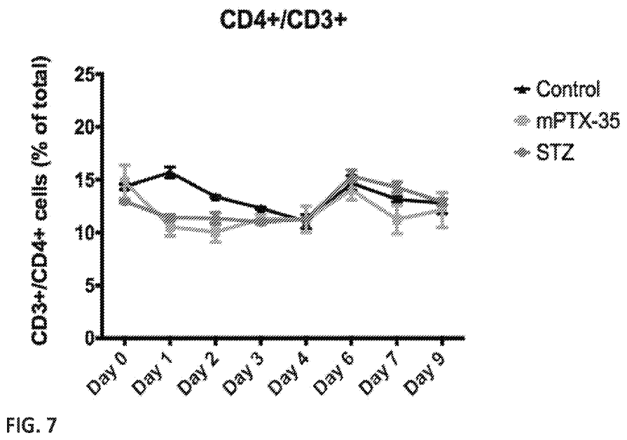

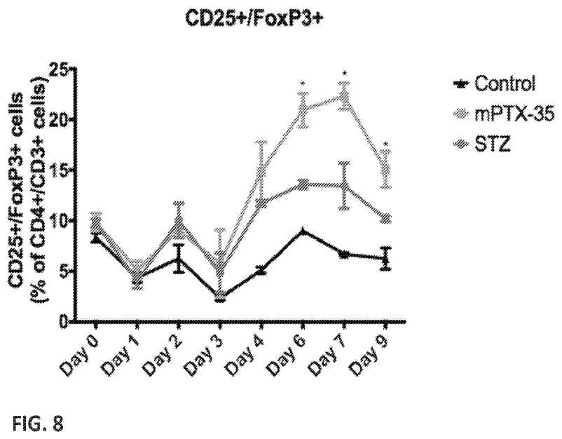

FIG. 5 illustrates CD4+/FoxP3+ cells (% of CD4+/CD3+ cells) in a murine model, in the group administered mPTX-35 (n=2), the group administered STZ (n=2), and in the control group (n=2). As shown, the administration of mPTX-35 led to the increased production of T regs (CD4+/FoxP3+ cells), relative to the control and STZ group, with the peak on day 6. FIG. 6 illustrates flow cytometry gating data for the control group (left panel, n=2) and the group administered mPTX-35 IgG1 (right panel, n=2). In each panel of FIG. 6, the top graph shows CD3+CD4+FoxP3+ on day 0 of the study, and the bottom graph shows CD3+CD4+FoxP3+ on day 6 of the study. FIG. 7 illustrates CD3+/CD4+ cells (% of total cells) in the murine model, in the group administered mPTX-35 (n=2), the group administered STZ (n=2), and in the control group (n=2). The results in FIG. 7 illustrate that the effect of mPTX-35 is corroborated, i.e. the effect of mPTX-35 is specific for CD4+/FoxP3+ population and no significant changes in the overall population of T cells is observed. FIG. 8 shows CD25+/FoxP3+ cells as percent of CD4+/CD3+ cells in the group administered mPTX-35 (n=2), the group administered STZ (n=2), and in the control group (n=2). As shown, administration of mPTX-35 results in the expansion of CD25+/FoxP3+population.

Results shown in FIGS. 4-8 corroborate the effect of mPTX-35 IgG1 on Tregs expansion and show that the effect is specific for CD4+/FoxP3+ population. Accordingly, the present results demonstrate that mPTX-35 can be used to manipulate Tregs for treatment and prevention of immune-related diseases and conditions, such as diabetes, graft-versus-host disease (GVHD) and transplant rejection.

### Example 3: Effect of Anti-TNF Receptor Superfamily Member 25 (TNFRSF25) Monoclonal Chimeric Agonistic Antibody 4C12 on Treg Expansion and Graft Survival

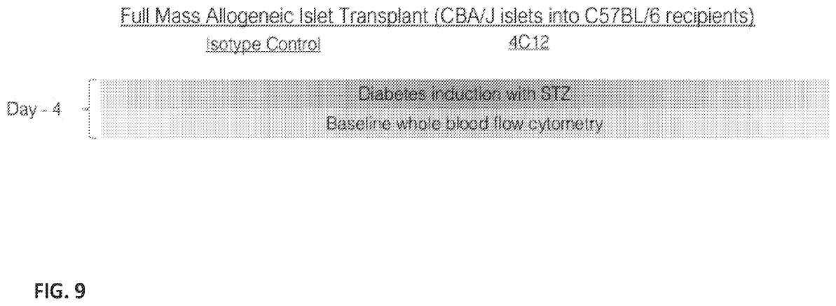

In the present study, the effect of 4C12 on regulatory T cells (Tregs) expansion and the role of 4C12 in islet transplantation in vivo were assessed, using a murine model. FIGS. 9, 10, 11A -11L, 12A -12I, 13A -13C, 14A, 14B, 15A -15D, 16A -16H, 17A, 17B, and 18A -18D demonstrate that administration of 4C12 increases the expansion of regulatory T cells, delays allograft rejection, and improves allograft survival.

Administration of 4C12 Leads to Pronounced Treg Expansion

FIG. 9 illustrates generally a design of experiments for this example to determine the role of the mouse-human anti-TNFRSF25 monoclonal chimeric agonistic antibody (4C12) in Teg expansion and in islet transplantation in vivo. An objective of this study was to determine whether administration of anti-TNFRSF25 agonist (4C12) can induce the proliferation of regulatory T cells in vivo in a murine model. In the experiments of this example, STZ is used to induce experimental diabetes in mice and study islet allotransplantation from BALB/c donor mice to C57BL/6 recipient mice. Recipient mice were injected with either the 4C12 antibody or with saline (control) to study whether the 4C12 antibody can delay allograft rejection and improve allograft survival. In this study, STZ and 4C12 were administered concomitantly four days before delivery (also referred to as “injection”) of islet transplants, to avoid prolonged hyperglycemia before transplantation. In this way, the artificial model was created by subjecting it to the diabetes-inducing challenge.

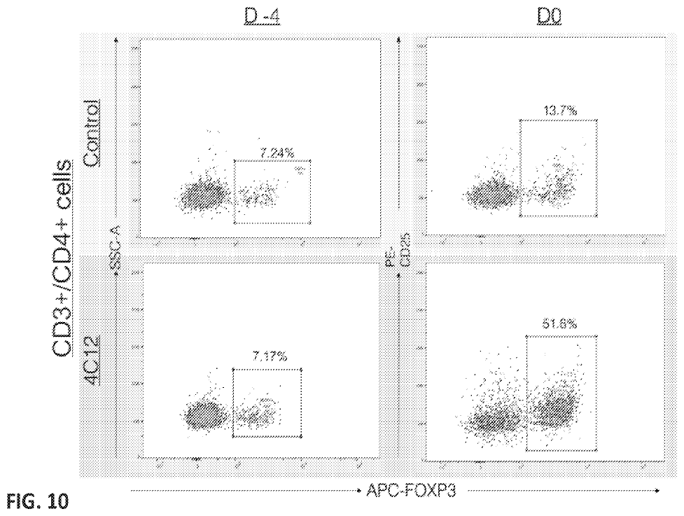

FIG. 10 illustrates CD3+/CD4+ cells (% of total cells) on day -4 (left panel), and day -0 (right panel), in the murine group administered saline (control; top panels), and the group administered the 4C12 antibody (bottom panels). The results in FIG. 10 illustrate the increase in production and expansion of T regulatory cells when the 4C12 antibody is administered.

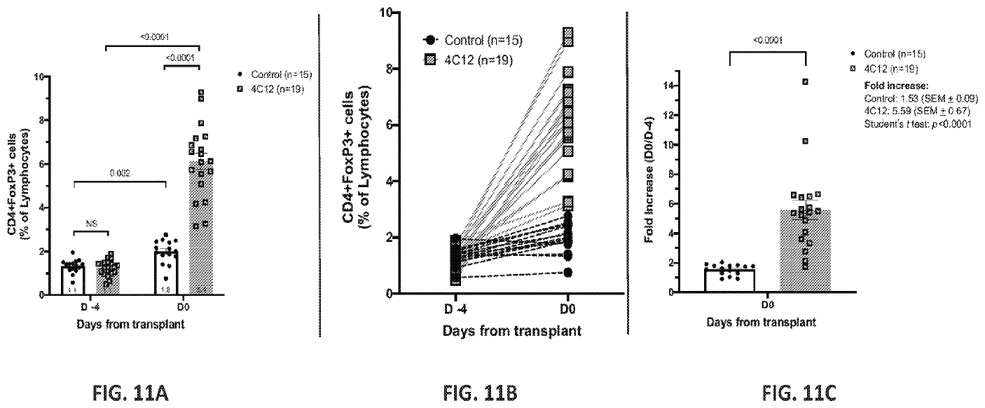

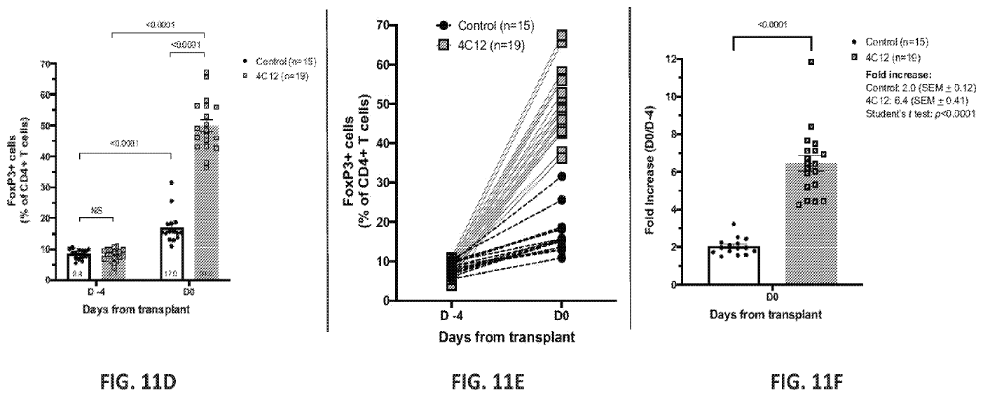

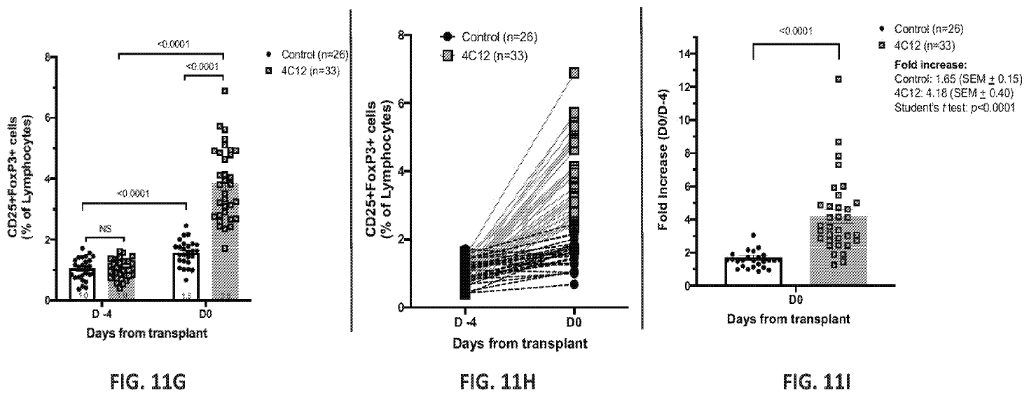

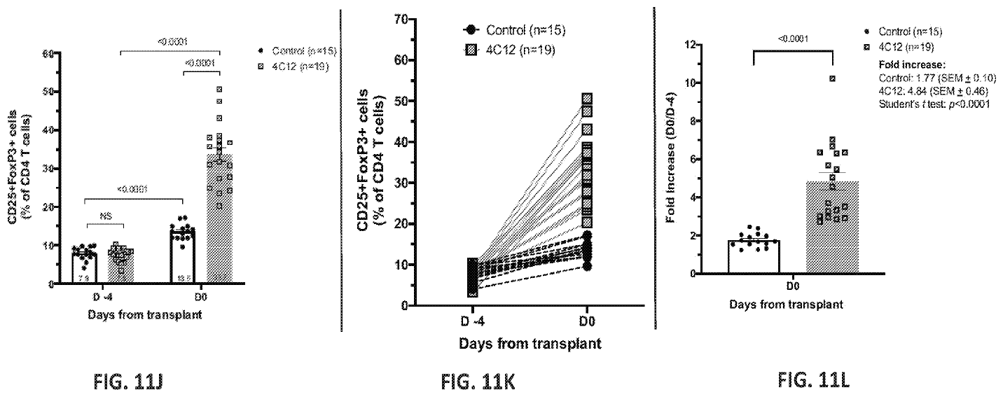

FIGS. 11A-11L further show the increase in production and expansion of T regulatory cells (Tregs) when the 4C12 antibody is administered, as measured by CD4+FoxP3+ cells as a percentage of lymphocytes (FIGS. 11A, 11B, and 11C), FoxP3+ cells as a percentage of CD4+ T cells (FIGS. 11D, 11E, and 11F), CD25+FoxP3+ cells as a percentage of lymphocytes (FIGS. 11G, 11H, and 11L), and CD25+FoxP3+ cells as a percentage of CD4+ T cells (FIGS. 11J, 11K, and 11L). Data are presented as means and standard errors of the mean (SEM).

Groups were compared using paired t tests to assess within group differences and unpaired t tests to assess between group differences.

FIGS. 11A, 11B, and 11C illustrate results as CD4+FoxP3+ cells (a percentage of lymphocytes), where the results were obtained using fifteen control mice (n=15) and 19 4C12 experimental mice (n=19). In FIG. 11A, CD4+FoxP3+ cells are shown as a percentage of lymphocytes at day -4 (“D -4”) from transplant (control experimental group, left bar; 4C12 experimental group, right bar), and at day 0 (“DO”) of transplant (control experimental group, left bar; 4C12 experimental group, right bar. In FIG. 11B, the increase in CD4+FoxP3+ cells is shown as a percentage of lymphocytes at day -4 from transplant, and at day 0 of transplant. In FIG. 11C, the fold increase (“DO” from transplant/“D-4” from transplant) in CD4+FoxP3+ cells as a percentage of lymphocytes is shown (control experimental group, left bar; 4C12 experimental group, right bar). FIG. 11C shows the fold increases of 1.53 and 5.59 for the control and 4C12 groups, respectively.

FIGS. 11D, 11E, and 11F illustrate results as FoxP3+ cells (a percentage of CD4+ T cells), where the results were obtained using fifteen control mice (n=15) and 19 4C12 experimental mice (n=19). In FIG. 11D, FoxP3+ cells are shown as a percentage of CD4+ T cells at day -4 from transplant (control experimental group, left bar; 4C12 experimental group, right bar), and at day 0 of transplant (control experimental group, left bar; 4C12 experimental group, right bar). In FIG. 11E, the increase in FoxP3+ cells is shown as a percentage of CD4+ T cells at day -4 from transplant, and at day 0 of transplant. In FIG. 11F, the fold increase (“DO” from transplant/“D-4” from transplant) in FoxP3+ cells as a percentage of CD4+ T cells is shown (control experimental group, left bar; 4C12 experimental group, right bar). FIG. 11F shows the fold increases of 2.0 and 6.4 for the control and 4C12 groups, respectively.

FIGS. 11G, 11H, and 11I illustrate results as CD25+FoxP3+ cells (a percentage of lymphocytes), where the results were obtained using twenty-six control mice (n=26) and 33 4C12 experimental mice (n=33). In FIG. 11G, CD25+FoxP3+ cells are shown as a percentage of lymphocytes at day -4 from transplant (control experimental group, left bar; 4C12 experimental group, right bar), and at day 0 of transplant (control experimental group, left bar; 4C12 experimental group, right bar). In FIG. 11H, the increase in CD25+FoxP3+ cells is shown as a percentage of lymphocytes at day -4 from transplant, and at day 0 of transplant. In FIG. 11I, the fold increase (“DO” from transplant/“D-4” from transplant) in CD25+FoxP3+ cells as a percentage of lymphocytes is shown (control experimental group, left bar; 4C12 experimental group, right bar). FIG. 11I shows the fold increases of 1.65 and 4.18 for the control and 4C12 groups, respectively.

FIGS. 11J, 11K, and 11L illustrate results as CD25+FoxP3+ cells (a percentage of CD4+ T cells), where the results were obtained using fifteen control mice (n=15) and 19 4C12 experimental mice (n=19). In FIG. 11J, CD25+FoxP3+ cells are shown as a percentage of CD4+ T cells at day -4 from transplant (control experimental group, left bar; 4C12 experimental group, right bar), and at day 0 of transplant (control experimental group, left bar; 4C12 experimental group, right bar). In FIG. 11K, the increase in CD25+FoxP3+ cells is shown as a percentage of CD4+ T cells at day -4 from transplant, and at day 0 of transplant. In FIG. 11L, the fold increase (“DO” from transplant/“D-4” from transplant) in CD25+FoxP3+ cells as a percentage of CD4+ T cells is shown (control experimental group, left bar; 4C12 experimental group, right bar). FIG. 11L shows the fold increases of 1.77 and 4.84 for the control and 4C12 groups, respectively. As shown in FIGS. 11A-11L, the administration of 4C12 led to a significant Treg expansion, including both CD4+FoxP3+ populations and CD4+CD25+FoxP3+(double positive populations).

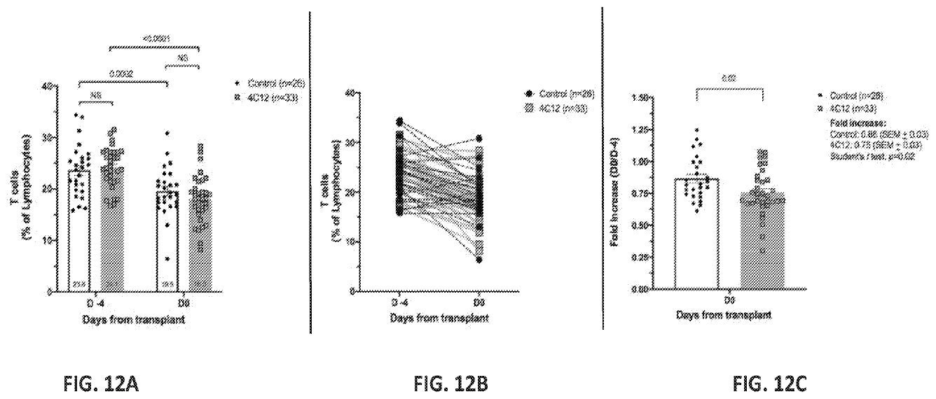

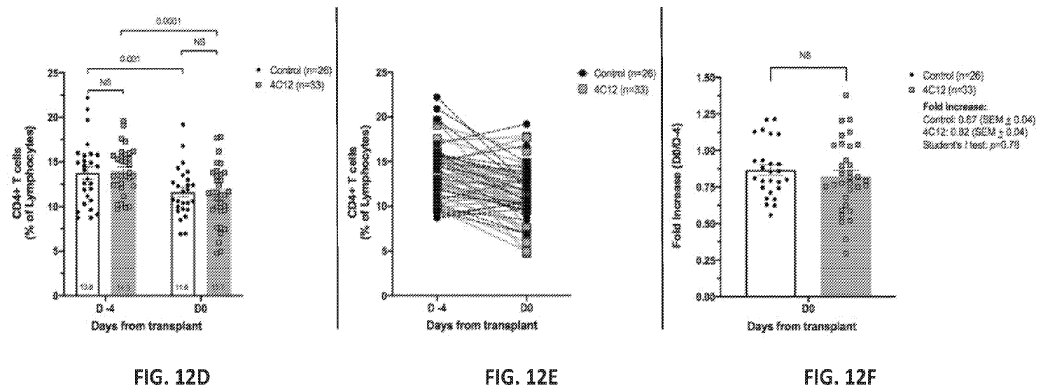

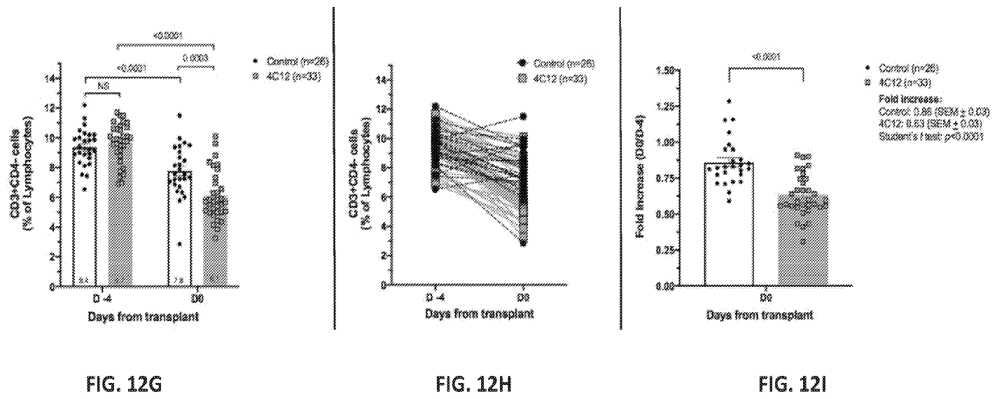

FIGS. 12A-12I show data for T cells as a percentage of lymphocytes (FIGS. 12A, 12B, and 12C), CD4+ T cells as a percentage of lymphocytes (FIGS. 12D, 12E, and 12F), and CD3+CD4− cells as a percentage of lymphocytes (FIGS. 12G, 12H, and 12I in the murine model of islet transplantation for the group administered 4C12 and STZ (n=33), and the control group (saline and STZ; n=26). The control group of mice received saline and STZ, and the experimental group of mice received the 4C12 antibody and STZ. In FIG. 12A, T cells are shown as a percentage of lymphocytes at day -4 from transplant (control experimental group, left bar; 4C12 experimental group, right bar), and at day 0 of transplant (control experimental group, left bar; 4C12 experimental group, right bar). In FIG. 12B, the decrease in T cells is shown as a percentage of lymphocytes at day -4 from transplant, and at day 0 of transplant, In FIG. 12C, the fold increase (“DO” from transplant/“D-4” from transplant) in T cells as a percentage of lymphocytes is shown (control experimental group, left bar; 4C12 experimental group, right bar). In FIG. 12D, CD4+ T cells are shown as a percentage of lymphocytes at day -4 from transplant (control experimental group, left bar; 4C12 experimental group, right bar), and at day 0 of transplant (control experimental group, left bar; 4C12 experimental group, right bar). In FIG. 12E, the decrease CD4+ T cells are shown as a percentage of lymphocytes at day -4 from transplant, and at day 0 of transplant. In FIG. 12F, the fold increase (“DO” from transplant/“D-4” from transplant) in CD4+ T cells as a percentage of lymphocytes is shown (control experimental group, left bar; 4C12 experimental group, right bar). In FIG. 12G, CD3+CD4− cells are shown as a percentage of lymphocytes at day -4 from transplant (control experimental group, left bar; 4C12 experimental group, right bar), and at day 0 of transplant (control experimental group, left bar; 4C12 experimental group, right bar). In FIG. 12H, the decrease in CD3+CD4− cells is shown as a percentage of lymphocytes at day -4 from transplant, and at day 0 of transplant. In FIG. 12I, the fold increase (“DO” from transplant/“D-4” from transplant) in CD3+CD4− cells as a percentage of lymphocytes is shown (control experimental group, left bar; 4C12 experimental group, right bar). The results demonstrate that presence of STZ leads to a mild T-cell depletion, a mild CD4+ T cell depletion, and a mild CD4− T-cell depletion. The results shown in FIGS. 12A-12I also demonstrate that the effect of the 4C12 antibody is specific for the CD4+/FoxP3+ population, and no significant changes in the overall population of T cells were observed.

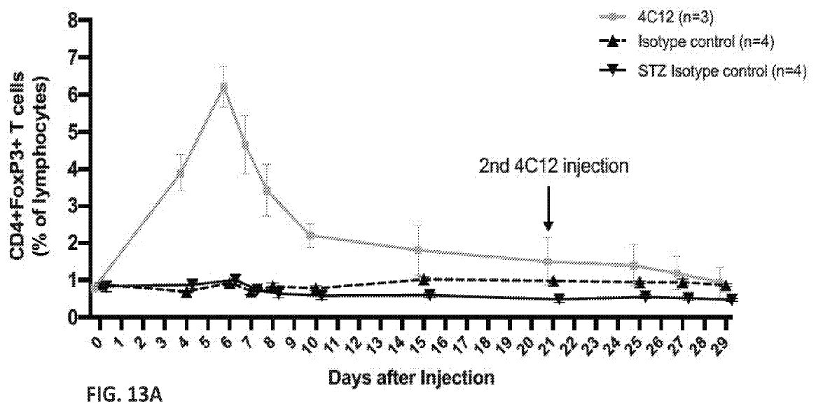

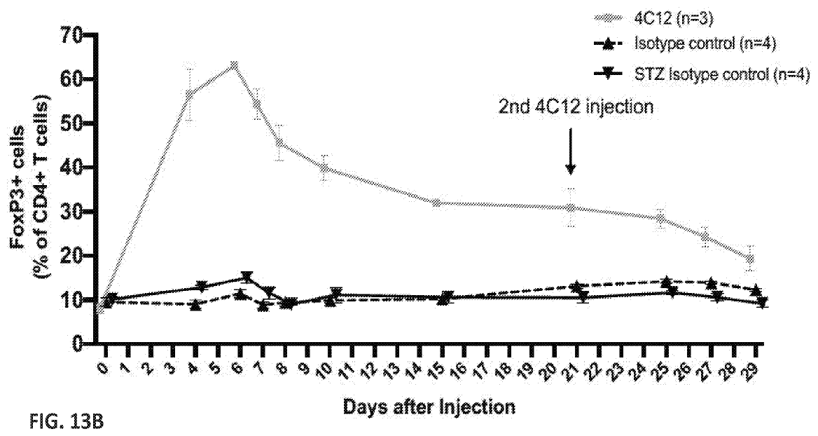

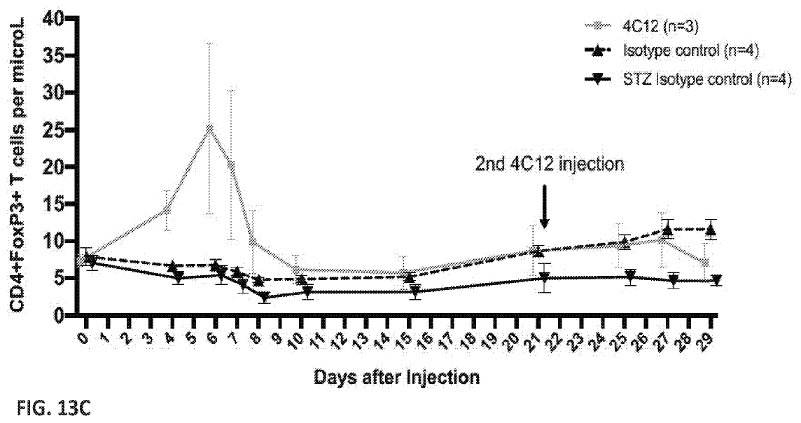

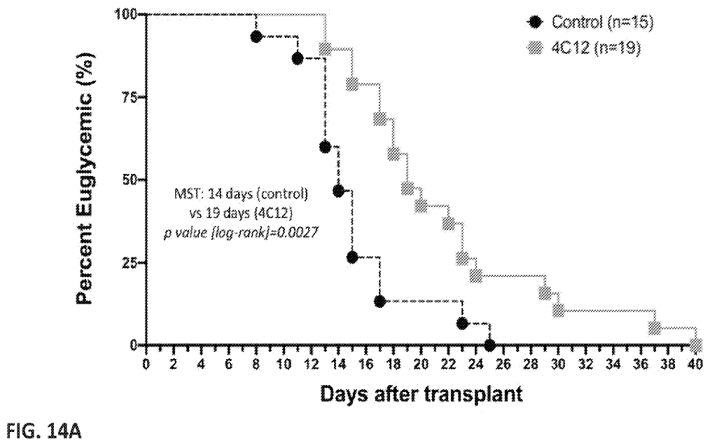

In the present study, flow cytometry analysis was conducted at different time points to characterize the kinetics of the Treg response to 4C12 administration. Thus, FIGS. 13A, 13B, and 13C illustrate the effect of 4C12 on Tregs, as Treg curves at different time points; the second 4C12 injection is shown, at day 21 after transplant delivery (injection). The results are shown for 4C12 group (n=3), Isotype control group (n=4), and STZ Isotype control group (n=4). FIG. 13A illustrates the effect of 4C12 on percentage of CD4+FoxP3+ T cells (% of lymphocytes) vs. days after injection. FIG. 13B illustrates the effect of 4C12 on percentage of FoxP3+ T cells (% of CD4 T cells) vs. days after injection. FIG. 13C illustrates the effect of 4C12 on CD4+FoxP3+ T cells (absolute number of T cells per microliter) vs. days after injection. Accordingly, in these experiments, an expansion in the absolute number of Tregs per microliter was corroborated.

Administration of 4C12 Prolongs Graft Survival

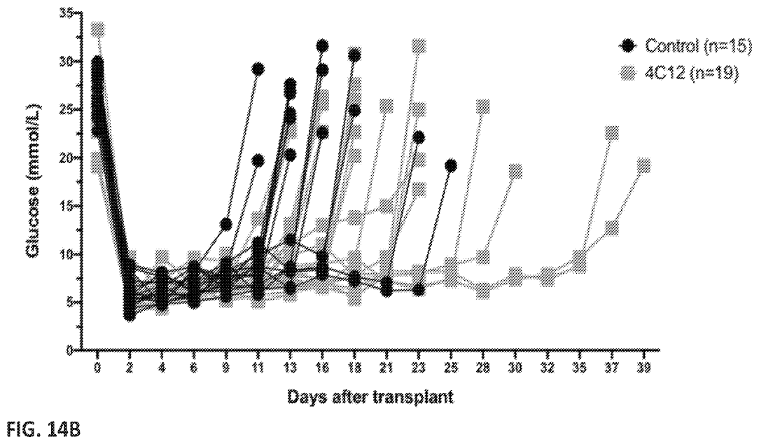

FIGS. 14A and 14B are graphs that show that administration of 4C12 antibody leads to delayed acute allograft rejection. FIGS. 14A and 14B illustrate effect of 4C12 on graft survival, showing delayed acute allograft rejection in mice administered 4C12 (n=19) as compared to control mice (n=15). FIG. 14A shows Kaplan-Meier graft survival estimates, for a percent of euglycemic mice over 40 days after transplant, where the mice in the control group had a median graft survival time (“MST”) of 14 days, as compared to the mice treated with 4C12 that had a MST of 19 days (log-rank test, p value=0.0027). FIG. 14B shows concentration of glucose (mmol/L) over 40 days after transplant.

Treg Expansion is Correlated with Graft Survival

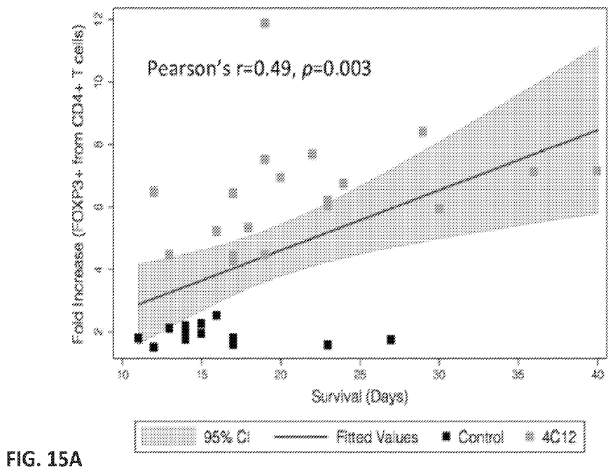

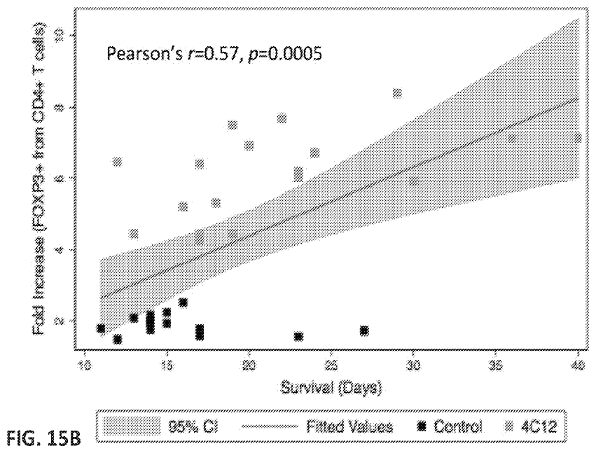

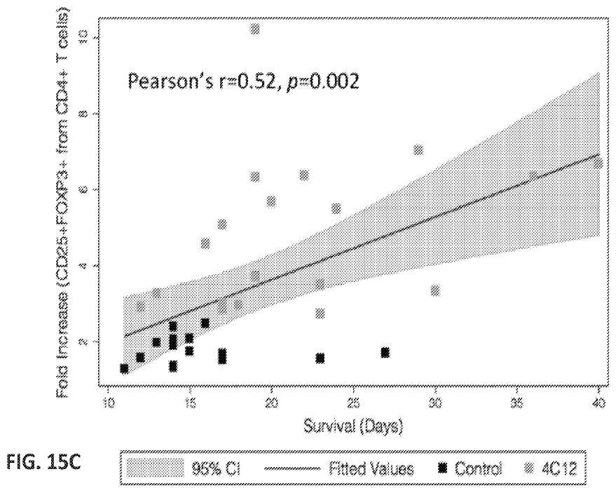

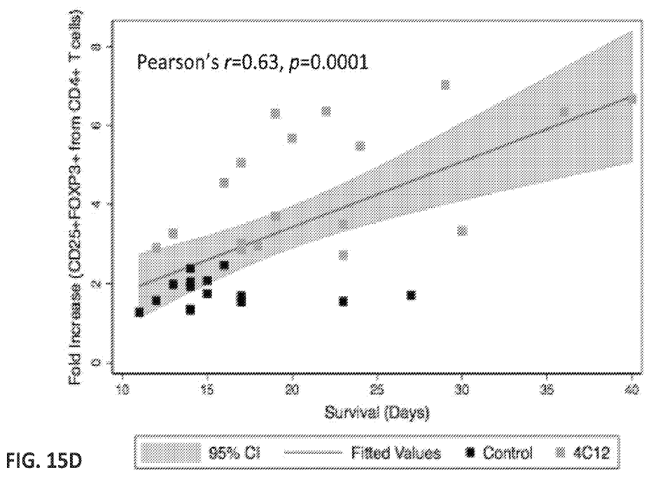

Further, in the present study, it was shown that Treg expansion is correlated with graft survival, as was shown by experiments in which the fold increase in Tregs (percentage at day 4/day 0) was correlated with graft survival. A significant correlation of Treg expansion with the fold increase in CD4+FOXP3+ and CD4+CD25+FOXP3+ cells was observed. FIGS. 15A-15D show correlation between Treg expansions and islet graft survival, illustrating that Treg fold increase has a moderate correlation with graft survival. FIG. 15A shows the fold increase of FoxP3+ cells from CD4+ T cells for mice that had the 4C12 antibody administered compared to control mice (Pearson's r=0.49, p=0.003). FIG. 15B shows the fold increase of FoxP3+ cells from CD4+ T cells for mice that had the 4C12 antibody administered compared to control mice (Pearson's r=0.57, p=0.0005). FIG. 15C shows the fold increase of CD25+FoxP3+ cells from CD4+ T cells for mice that had the 4C12 antibody administered compared to control mice (Pearson's r=0.52, p=0.002). FIG. 15D shows the fold increase of CD25+FoxP3+ cells from CD4+ T cells for mice that had the 4C12 antibody administered compared to control mice (Pearson's r=0.63, p=0.0001).

Administration of 4C12 Leads to Increased Serum Levels of IL-5

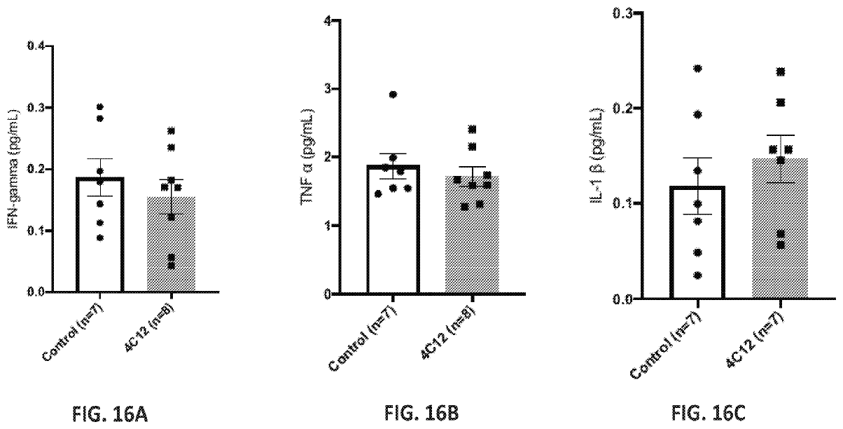

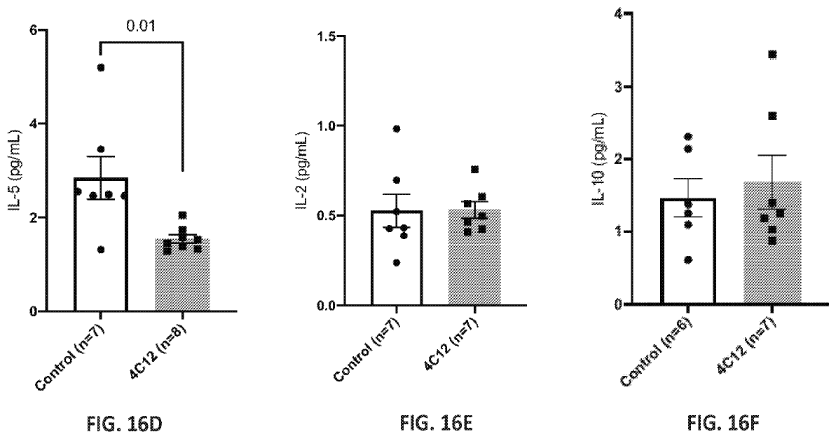

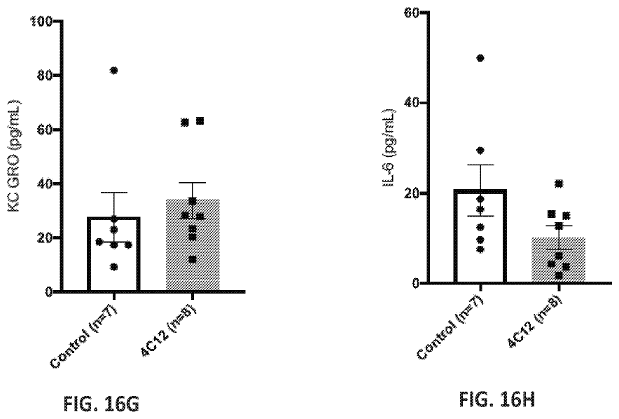

Serum levels of several Treg-related cytokines and biomarkers were measured. FIGS. 16A-16H show the effect of 4C12 on levels of several cytokines and biomarkers early after transplant, in pictogram per milliliter (pg/mL). The levels of IFN-γ (FIG. 16A), TNF-α (FIG. 16B), IL-1β (FIG. 16C), IL-5 (FIG. 16D), IL-2 (FIG. 16E), IL-10 (FIG. 16F), KC GRO (FIG. 16G), and IL-6 (FIG. 16H) are shown (control experimental group, left bar; 4C12 experimental group, right bar). A significant difference was observed only for IL-5 serum levels which were significantly lower in the 4C12 group.

Systemic Treg Expansion is not Correlated with Intra-Graft Treg Infiltration

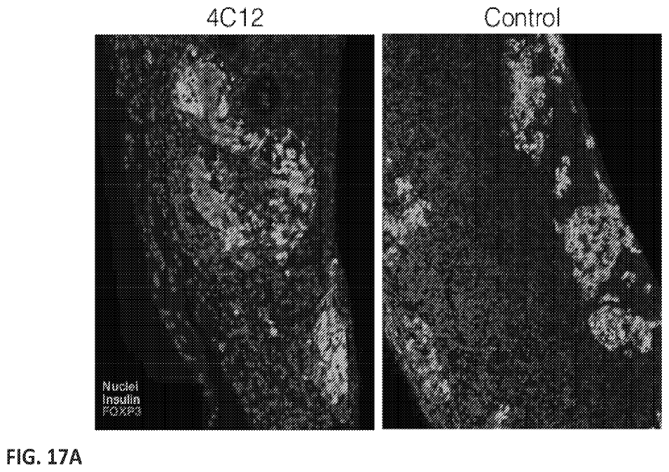

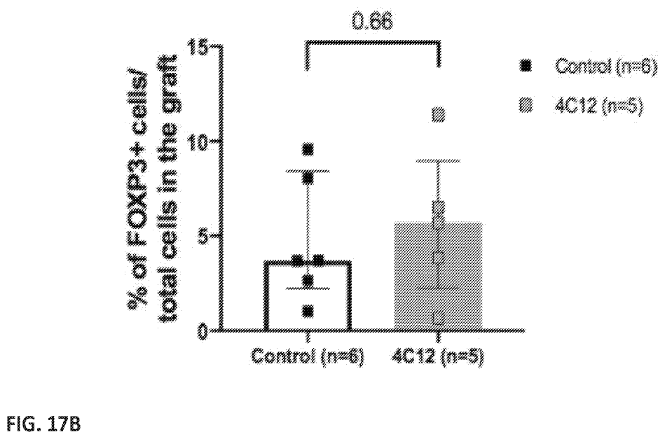

An acute graft rejection study was conducted in which an islet graft was obtained from nephrectomy of mice at day 7 post-transplantation. Immunohistochemistry analysis of the islet graft was then performed. FIGS. 17A and 17B illustrate results obtained using immunohistochemistry analysis of Treg infiltration within the islet graft, in the acute graft rejection study. FIG. 17A shows immunohistochemistry for 4C12 mice (left panel) and control mice (right panel). In FIG. 17A, the nuclei are shown in blue, insulin is shown in green, and FoxP3+ cells are shown in red. FIG. 17B shows a percentage of FoxP3+ cells out of total cells in the graft, the results are shown for the control mice group (n=6) and for mice administered 4C12 (n=5). No significant differences in the number of Treg cells in the graft was observed, with median percentage of FoxP3+ T cells per graft of 5.7% (IQR: 2.3-9.0) vs 3.7 (IQR: 2.2-8.4) in the 4C12 and control groups, respectively (p=0.66).

Tregs Numbers in the Control and 4C12 Groups

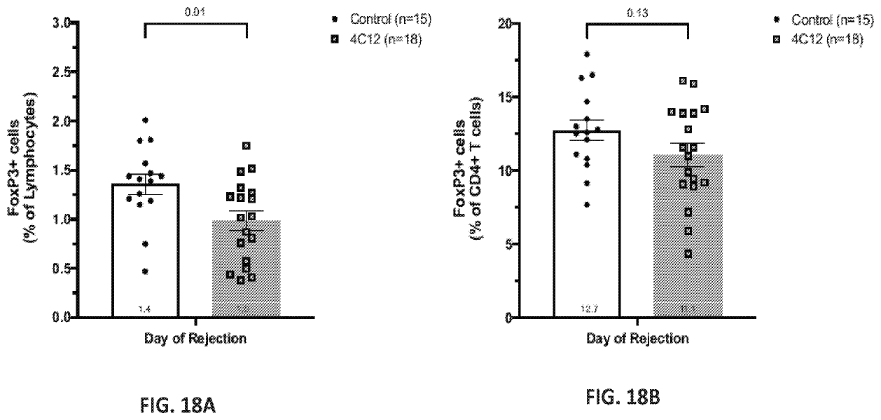

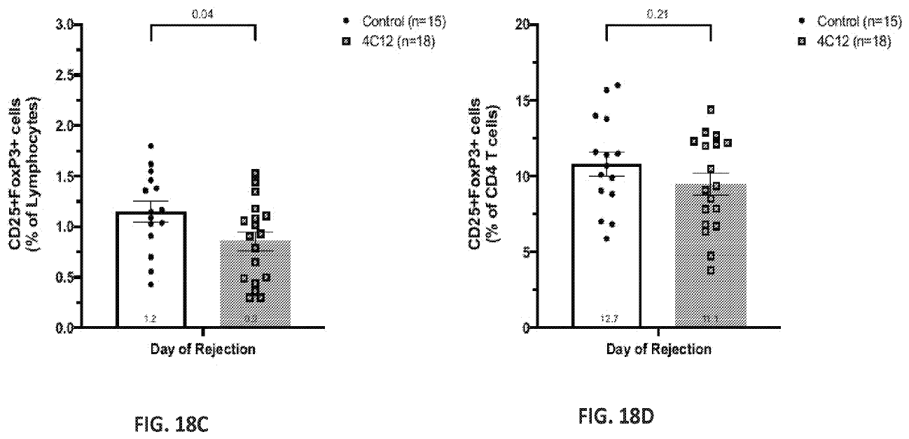

FIGS. 18A-18D illustrate Treg percentages at the day of rejection, for control mice group (n=15) and mice group administered 4C12 (n=18). FIG. 18A shows FoxP3+ cells (% of lymphocytes) for the control mice group (left bar) and for the mice group administered 4C12 (right bar). FIG. 18B shows FoxP3+ cells (% of CD4+T cells) for the control mice group (left bar) and for the mice group administered 4C12 (right bar). FIG. 18C shows CD25+FoxP3+ cells (% of lymphocytes) for the control mice group (left bar) and for the mice group administered 4C12 (right bar). FIG. 18D shows CD25+FoxP3+ cells (% of CD4+ T cells) for the control mice group (left bar) and for the mice group administered 4C12 (right bar). As shown in FIGS. 18A-18D, it was observed that the percentage of Tregs from the total number of lymphocytes was significantly lower in the 4C12 group. At the same time, the percentage of Tregs from the CD4+ T cells was similar between groups.

The results of these experiments show that administration of the 4C12 antibody results in an increase in production and expansion of T regulatory cells, which delays allograft rejection and leads to an improvement in allograft survival.

### Example 4: Effect of Anti-TNF Receptor Superfamily Member 25 (TNFRSF25) Monoclonal Chimeric Agonistic Antibody mPTX-35 on Treg Expansion and Graft Survival

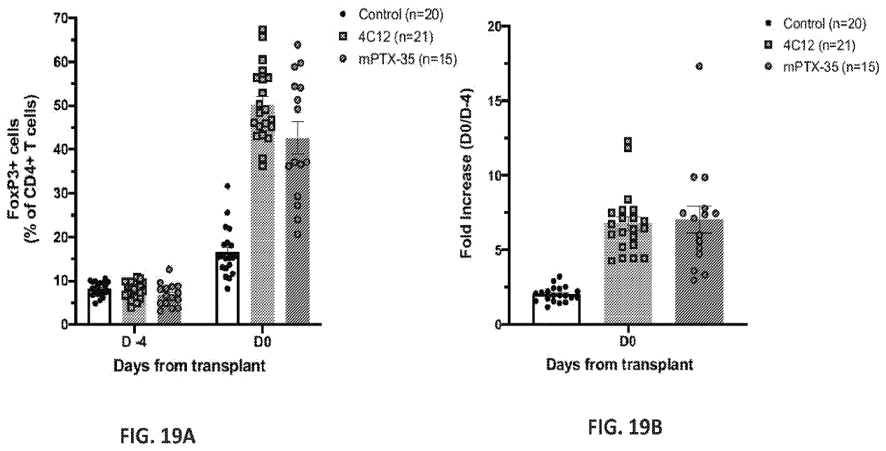

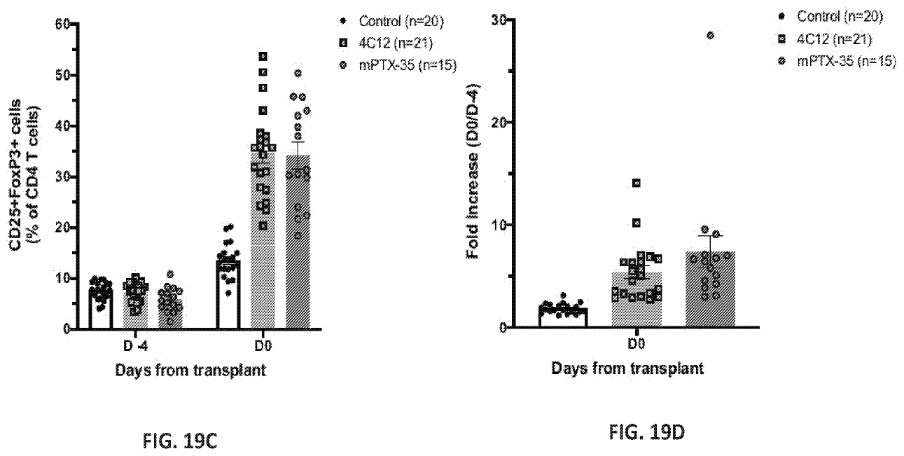

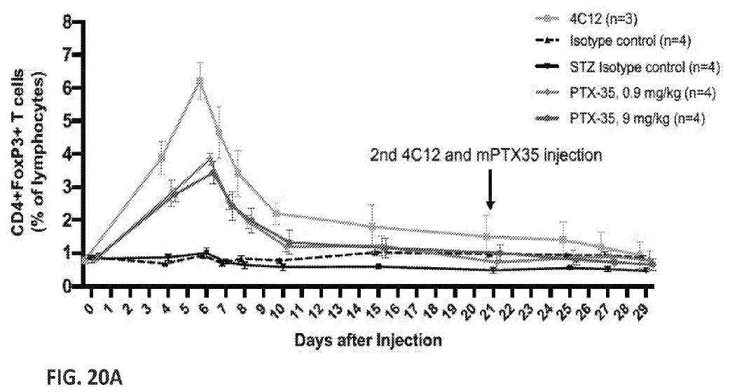

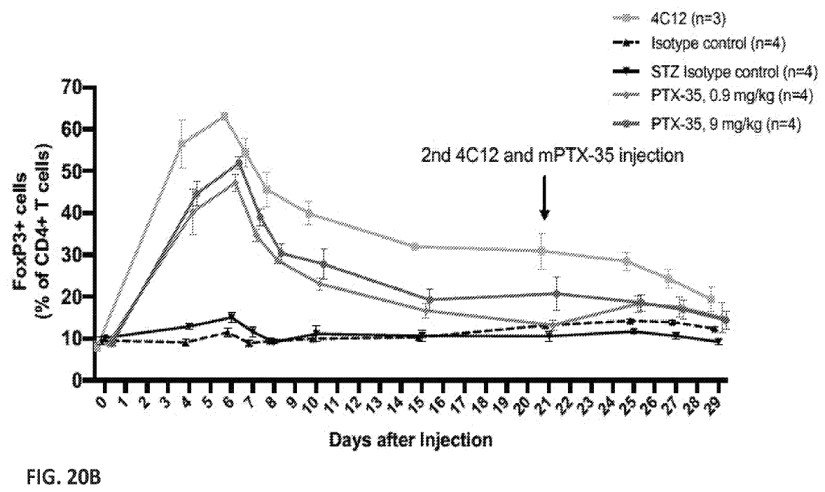

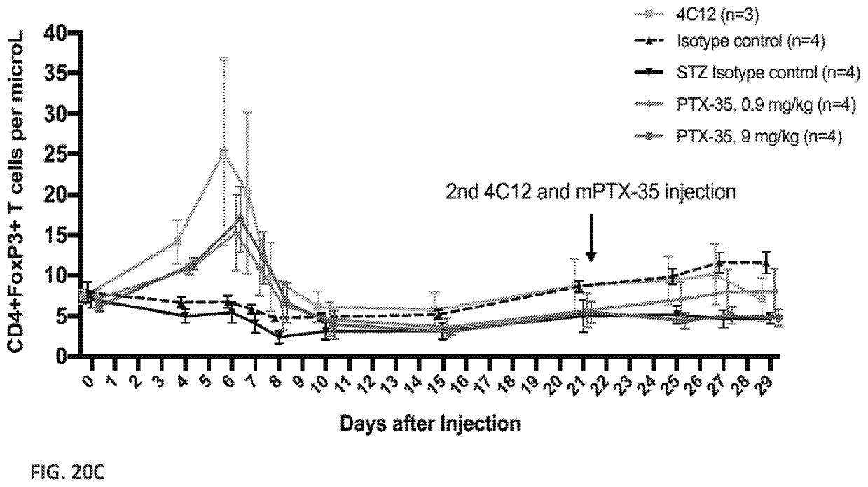

In the present study, the effect of mPTX-35 on regulatory T cells expansion and the role of mPTX-35 in islet transplantation in vivo were assessed, using a murine model. FIGS. 19A-19D, 20A -20B, 21, and 22 demonstrate that administration of mPTX-35 leads to pronounced Treg expansion and prolongs survival of a graft, such as an islet graft.

Administration of mPTX-35 Leads to Increased Treg Expansion

In this study, the effect of mPTX-35 on Treg expansion was assessed. In the experiments, mPTX-35 was administered four days before transplantation, concomitantly with STZ. This led to a significant expansion of the Treg populations, including both CD4+FoxP3+ populations and CD4+CD25+FoxP3+(double positive populations). Thus, FIGS. 19A-19D illustrate effect of 4C12 and mPTX-35 on in vivo Treg expansion. mPTX-35 was administered four days before transplantation, concomitantly with STZ, which led to a significant expansion of the Treg populations, including both CD4+FoxP3+ populations and CD4+CD25+FoxP3+(double positive populations). The results were obtained using a control experimental mice group (n=20), a 4C12 experimental mice group (n=21), and an mPTX-35 experimental mice group (n=15). FIG. 19A shows FoxP3+ cells as a percentage of CD4+ cells at day -4 (“D -4”) from transplant (control experimental group, left bar; 4C12 experimental group, middle bar; mPTX35 experimental group, right bar), and at day 0 (“DO”) of transplant (control experimental group, left bar; 4C12 experimental group, middle bar; mPTX-35 experimental group, right bar). FIG. 19B shows the fold increase (“DO” from transplant/“D-4” from transplant) in FoxP3+ cells as a percentage of CD4+ cells (control experimental group, left bar; 4C12 experimental group, middle bar; mPTX-35 experimental group, right bar). FIG. 19C shows CD25+FoxP3+ cells as a percentage of CD4+ cells at day -4 (“D -4”) from transplant (control experimental group, left bar; 4C12 experimental group, middle bar; mPTX35 experimental group, right bar), and at day 0 (“DO”) of transplant (control experimental group, left bar; 4C12 experimental group, middle bar; mPTX-35 experimental group, right bar). FIG. 19D shows the fold increase (“DO” from transplant/“D-4” from transplant) in CD25+FoxP3+ cells as a percentage of CD4+ cells (control experimental group, left bar; 4C12 experimental group, middle bar; mPTX-35 experimental group, right bar).

Further, in the present study, a flow cytometry analysis was conducted at different time points to characterize the kinetics of the Treg response. FIGS. 20A-20C show results of the flow cytometry analysis at different time points in diabetic mice, for 4C12 group (n=3), Isotype control group (n=4), STZ Isotype control group (n=4), PTX-35, 0.9 mg/kg (n=4), and PTX-35, 9 mg/kg (n=4). In this study, mPTX-35 was administered four days before transplantation, concomitantly with STZ. The second 4C12 and mPTX-35 injections were administered at day 21 post-transplant delivery (injection). FIG. 20A shows CD4+FoxP3+ T cells as a percentage of lymphocytes versus a number of days after the transplant delivery (injection). For reference, in FIG. 20A, on day 6, the 4C12 group has about 6.2, the mPTX-35 (0.9 mg/kg) group has about 3.8, the mPTX-35 (9 mg/kg) group has about 3.5, and the Isotype and STZ Isotype control groups have about 1 percent of CD4+FoxP3+ T cells, as a percent of lymphocytes. FIG. 20B shows FoxP3+ T cells as a percentage of CD4+ T cells versus a number of days after the transplant delivery (injection). For reference, in FIG. 20B, on day 6, the 4C12 group has about 62, the mPTX-35 (9 mg/kg) group has about 50, the mPTX-35 (0.9 mg/kg) group has about 48, the STZ Isotype control group has about 12, and the Isotype control group has about 10 percent of FoxP3+ T cells, as a percent of CD4+ T cells. FIG. 20C shows CD4+FoxP3+ T cells, as the absolute number of T cells per microliter versus a number of days after the transplant delivery (injection). For reference, in FIG. 20C, on day 6, the 4C12 group has about 25, the mPTX-35 (9 mg/kg) group has about 14, the mPTX-35 (0.9 mg/kg) group has about 14.5, the Isotype control group has about 6, and the STZ Isotype control group has about 5 CD4+FoxP3+ T cells per micro Liter. In these experiments, an expansion in the absolute numbers of Tregs per microliter was corroborated.

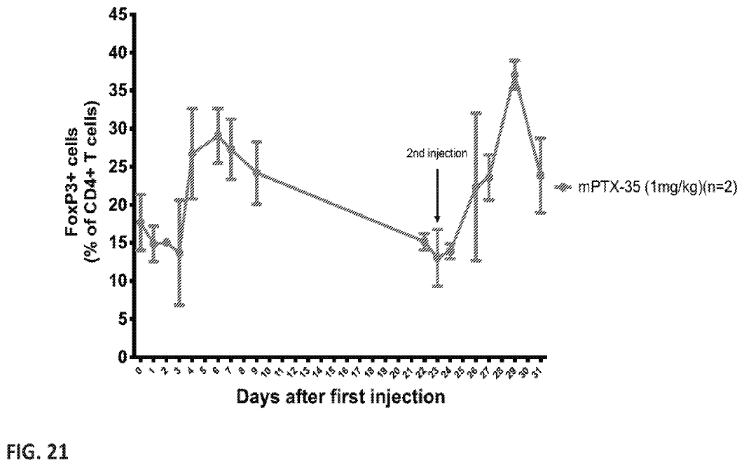

Interestingly, as illustrated in FIGS. 20A-20C, the lack of Treg re-expansion with a 2nd dose of mPTX-35 was observed, both using 0.9 mg/kg and 9 mg/kg mPTX-35 doses. At the same time, as shown in FIG. 21, illustrating a Treg curve for non-diabetic mice, a 2nd peak of Treg expansion was observed after the 21-days resting period. Without wishing to be bound by the theory, it may be explained by the use of non-diabetic mice in the study of FIG. 21 and the use of diabetic mice in the experiments of FIGS. 20A-20C. In FIG. 21, the graph shows FoxP3+ cells as a percentage of CD4+ cells versus a number of days after the first injection, for non-diabetic mice (n=2) administered mPTX-35 (1 mg/kg).

Administration of mPTX-35 Prolongs Graft Survival

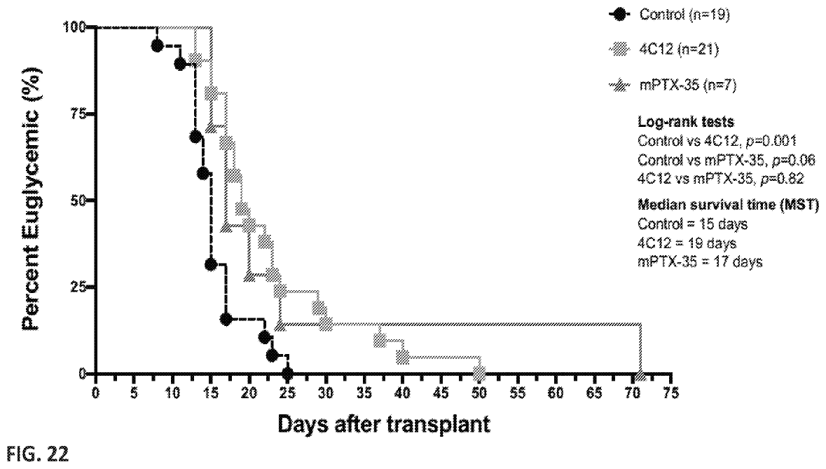

FIG. 22 show Kaplan-Meier graft survival estimates demonstrating that mPTX-35 administration results in a statistically significant delay in acute rejection thereby increasing graft survival. The results are shown for control group (n=19), 4C12 group (n=21), and mPTX-35 group (n=7). Mice in the control group had a median graft survival (MST) of 15 days, as compared to the mice group administered 4C12 (MST of 19 days, log-rank test, p=0.001) and the mice group administered mPTX-35 (MST of 17 days, log-rank test, p=0.06). As shown in FIG. 22, a statistically significant delay in acute islet graft rejection was observed. Mice in the control group had a median graft survival (MST) of 15 days, as compared to mice treated with 4C12 (MST of 19 days, log-rank test=0.001) and mice treated with mPTX-35 (MST of 17 days, log-rank test=0.06). No significant differences were observed between mice treated with 4C12 and mPTX35.

The results of these experiments show that administration of the mPTX-35 antibody results in an increase in production and expansion of T regulatory cells that delays graft rejection and leads to an improvement in allograft survival. Also, the effect of mPTX-35 antibody on expansion of Tregs and delaying graft rejection and increasing graft survival and, surprisingly, is substantially the same as the effect of the 4C12 antibody on expansion of Tregs and delaying graft rejection and increasing graft survival.

### OTHER EMBODIMENTS

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

### INCORPORATION BY REFERENCE

All patents and publications referenced herein are hereby incorporated by reference in their entireties. The publications discussed herein are provided solely for their disclosure prior to the filing date of the present application. Nothing herein is to be construed as an admission that the present invention is not entitled to antedate such publication by virtue of prior invention. As used herein, all headings are simply for organization and are not intended to limit the disclosure in anyway.

What is claimed is:

1. A method for treating or preventing diabetes, prediabetes, and/or glucose intolerance, comprising administering an effective amount of TNF Receptor Superfamily Member 25 (TNFRSF25) agonistic antibody or antigen binding fragment thereof to a patient in need thereof.

2. The method of claim 1, wherein the patient suffers from insulin resistance.

3. The method of claim 1, wherein the patient is diagnosed with one or more of insulin resistance, prediabetes, impaired fasting glucose (IFG), impaired glucose tolerance (IGT), and acanthosis nigricans.

4. The method of any of the above claims, wherein the patient has cardiovascular disease or metabolic disease.

5. The method of any one of the above claims, wherein the patient has type 1 diabetes or type 2 diabetes.

6. The method of any one of the above claims, wherein the patient has gestational diabetes or steroid-induced diabetes.

7. The method of any one of the above claims, wherein the TNFRSF25 agonistic antibody or antigen binding fragment thereof is administered as a regimen that decreases blood glucose level, stimulates peripheral glucose disposal, and/or inhibits hepatic glucose production.

8. The method of any one of the above claims, wherein the patient has one or more of an average hemoglobin A1c value of more than about 10% and an average glucose of more than about 200 mg/dl (11 mmol/I) at the start of treatment with conventional diabetic therapy.

9. The method of claim 8, wherein the conventional diabetic therapy is insulin therapy and/or non-insulin diabetes agent therapy.

10. The method of any one of the above claims, wherein the TNFRSF25 agonistic antibody or antigen binding fragment administration is effective for providing average glucose of below about 200 mg/dl (11 mmol/I).

11. The method of any one of the above claims, wherein the TNFRSF25 agonistic antibody or antigen binding fragment administration is effective for providing average glycosylated hemoglobin levels (hemoglobin A1c) values of about 8% or less.

12. The method of any one of the above claims, wherein the patient does not experience an increase of insulin production upon TNFRSF25 agonistic antibody or antigen binding fragment administration.

13. The method of any one of the above claims, wherein the TNFRSF25 agonistic antibody or antigen binding fragment thereof is administered as an adjuvant therapy.

14. The method of any one of the above claims, wherein the patient is undergoing treatment with one or more of insulin or an insulin analog.

15. The method of claim 14, wherein the insulin analog is selected from a rapid acting or long acting insulin analog.

16. The method of claim 15, wherein the rapid acting insulin analog is lispro, aspart or glulisine.

17. The method of claim 15, wherein the long acting insulin analog is glargine or detemir.

18. The method of any one of the above claims, wherein the TNFRSF25 agonistic antibody or antigen binding fragment administration does not cause hypoglycemia.

19. The method of any one of the above claims, wherein the TNFRSF25 agonistic antibody or antigen binding fragment administration does not cause hypokalemia.

21. The method of any one of the above claims, wherein the TNFRSF25 agonistic antibody or antigen binding fragment thereof comprises:
(i) a heavy chain variable region comprising heavy chain CDR1, CDR2, and CDR3 sequences, wherein the heavy chain CDR1 sequence is GFTFSNHDLN (SEQ ID NO: 1), the heavy chain CDR2 sequence is YISSASGLISYADAVRG (SEQ ID NO: 2); and the heavy chain CDR3 sequence is DPAYTGLYALDF (SEQ ID NO: 3) or DPPYSGLYALDF (SEQ ID NO: 4); and
(ii) a light chain variable region comprising light chain CDR1, CDR2, and CDR3 sequences, wherein the light chain CDR1 sequence is TLSSELSWYTIV (SEQ ID NO: 5), the light chain CDR2 sequence is LKSDGSHSKGD (SEQ ID NO: 6), and the light chain CDR3 sequence is CGAGYTLAGQYGWV (SEQ ID NO: 7).

22. The method of claim 21, wherein the TNFRSF25 agonistic antibody or antigen binding fragment thereof further comprises variable region framework (FW) sequences juxtaposed between the CDRs according to the formula (FW1)-(CDR1)-(FW2)-(CDR2)-(FW3)-(CDR3)-(FW4), wherein the variable region FW sequences in the heavy chain variable region are heavy chain variable region FW sequences, and wherein the variable region FW sequences in the light chain variable region are light chain variable region FW sequences.

23. The method of claim 22, wherein the variable region FW sequences are human.

24. The method of any one of claims 1 to 23, wherein the TNFRSF25 agonistic antibody or antigen binding fragment thereof further comprises human heavy chain and light chain constant regions.

25. The method of claim 24, wherein the constant regions are selected from the group consisting of human IgG1, IgG2, IgG3, and IgG4.

26. The method of claim 25, wherein the constant regions are IgG1.

27. The method of claim 25, wherein the constant regions are IgG4.

28. The method of any one of claims 1 to 27, wherein the TNFRSF25 agonistic antibody or antigen binding fragment thereof comprises a heavy chain variable region having the amino acid sequence EVQLVESGGGLSQPGNSLQLSCEASGFTFSNHDLNWVRQAPGKGLEWVAYISSASGLISYADAVRGRFTISRDN AKNSLFLQMNNLKSEDTAMYYCARDPPYSGLYALDFWGQGTQVTVSS (SEQ ID NO: 8), or an amino acid sequence of at least about 85 to about 99% identity thereto.

29. The method of any one of claims 1 to 28, wherein the TNFRSF25 agonistic antibody or antigen binding fragment thereof comprises a light chain variable region having the amino acid sequence QPVLTQSPSASASLSGSVKLTCTLSSELSSYTIVWYQQRPDKAPKYVMYLKSDGSHSKGDGIPDRFSGSSSGAH RYLSISNVQSEDDATYFCGAGYTLAGQYGWVFGSGTKVTVL (SEQ ID NO: 9), or an amino acid sequence of at least about 85 to about 99% identity thereto.

30. The method of any of the above claims, wherein the treatment expands and/or selectively activates a population of Tregs in the patient.

31. The method of any of the above claims, wherein the patient is a recipient of a transplant.

32. The method of claim 31, wherein the transplant comprises an islet cell transplant.

33. The method of claim 31, wherein the transplant comprises a solid organ transplant.

34. The method of claim 33, wherein the solid organ transplant comprises a pancreas transplant.

35. A method for treating diabetes and/or glucose intolerance, comprising administering an effective amount of TNFRSF25 agonistic antibody or antigen binding fragment thereof to a patient in need thereof, wherein the patient is not receiving insulin therapy.

36. The method of claim 35, wherein the TNFRSF25 agonistic antibody or antigen binding fragment stimulates glucose uptake in the patient.

37. The method of claim 36, wherein the glucose uptake in mediated by glucose transporter type 4 (GLUT4).

38. The method of claim 36, wherein the glucose uptake is substantially in muscle or fat cells.

39. The method of any one of claims 35 to 38, wherein the patient is not receiving one or more of basal, preprandial, and postprandial insulin therapy.

40. The method of any one of claims 35 to 39, wherein the patient is not receiving basal insulin therapy but is receiving preprandial or postprandial insulin therapy.

41. The method of any one of claims 35 to 40, wherein the patient is not receiving preprandial or postprandial insulin therapy but is receiving basal insulin therapy.

42. The method of any one of claims 35 to 41, wherein the patient has not received insulin therapy in up to about 1 hour, or up to about 2 hours, or up to about 3 hours, or up to about 4 hours, or up to about 5 hours, or up to about 6 hours, or up to about 7 hours, or up to about 8 hours, or up to about 12 hours or up to about 16 hours or up to about 20 hours, or up to about 24 hours, up to about 2 days, up to about 3 days, up to about 4 days, up to about 5 days, up to about 6 days, up to about 7 days.

43. The method of any one of claims 35 to 42, wherein the patient has experienced one or more instances of lipodystrophy that is caused by injection.

44. The method of any one of claims 35 to 43, wherein the patient is afflicted with or is at risk of having hypokalemia.

45. The method of any one of claims 35 to 44, wherein the patient is afflicted with or is at risk of having an insulin allergy.

46. The method of any one of claims 35 to 45, wherein the patient is receiving one or more non-insulin diabetes agents selected from metformin (e.g. GLUCOPHAGE, GLUMETZA); sulfonylureas (e.g. glyburide (e.g. DIABETA, GLYNASE), glipizide (e.g. GLUCOTROL) and glimepiride (e.g. AMARYL)); thiazolidinediones (e.g. rosiglitazone (e.g. AVANDIA) and pioglitazone (e.g. ACTOS)); DPP-4 inhibitors (e.g. sitagliptin (e.g. JANUVIA), saxagliptin (e.g. ONGLYZA) and linagliptin (e.g. TRADJENTA)); GLP-1 receptor agonists (e.g. exenatide (e.g. BYETTA) and liraglutide (e.g. VICTOZA)); and SGLT2 inhibitors (e.g. canagliflozin (e.g. NVOKANA) and dapagliflozin (e.g. FARXIGA)).

47. The method of any one of claims 35 to 46, wherein the patient is not receiving one or more non-insulin diabetes agents selected from metformin (e.g. GLUCOPHAGE, GLUMETZA); Sulfonylureas (e.g. glyburide (e.g. DIABETA, GLYNASE), glipizide (e.g. GLUCOTROL) and glimepiride (e.g. AMARYL)); thiazolidinediones (e.g. rosiglitazone (e.g. AVANDIA) and pioglitazone (e.g. ACTOS)); DPP-4 inhibitors (e.g. sitagliptin (e.g. JANUVIA), saxagliptin (e.g. ONGLYZA) and linagliptin (e.g. TRADJENTA)); GLP-1 receptor agonists (e.g. exenatide (e.g. BYETTA) and liraglutide (e.g. VICTOZA)); and SGLT2 inhibitors (e.g. canagliflozin (e.g. NVOKANA) and dapagliflozin (e.g. FARXIGA)).

48. The method of any one of claims 35 to 47, wherein the TNFRSF25 agonistic antibody or antigen binding fragment is an adjuvant therapy to an insulin therapy and/or a non-insulin diabetes agent.

49. The method of any one of claims 35 to 48, wherein the TNFRSF25 agonistic antibody or antigen binding fragment is an insulin replacement therapy.

50. The method of any one of claims 35 to 49, wherein the insulin analog is selected from a rapid acting or long acting insulin analog.

51. The method of claim 50, wherein the rapid acting insulin analog is lispro, aspart or glulisine.

52. The method of claim 50, wherein the long acting insulin analog is glargine or detemir.

53. The method of any one of claims 35 to 52, wherein the patient has type 1 diabetes or type 2 diabetes.

54. The method of any one of claims 35 to 52, wherein the patient has gestational diabetes or steroid-induced diabetes.

55. The method of any one of claims 35 to 54, wherein the treatment comprises one or more of a decrease of the blood glucose level; stimulation of peripheral glucose disposal; and inhibition of hepatic glucose production.

56. The method of any one of claims 35 to 55, wherein the TNFRSF25 agonistic antibody or antigen binding fragment administration is effective for providing glycemic control.

57. The method of any one of claims 35 to 56, wherein the patient suffers from insulin resistance.

58. The method of any one of claims 35 to 57, wherein the patient is diagnosed with one or more of insulin resistance, prediabetes, impaired fasting glucose (IFG), impaired glucose tolerance (IGT), and acanthosis nigricans.

59. The method of any one of claims 35 to 58, wherein the patient has cardiovascular disease or metabolic disease.

60. The method of any one of claims 35 to 59, wherein the TNFRSF25 agonistic antibody or antigen binding fragment is administered as a regimen that decreases blood glucose level; stimulates peripheral glucose disposal; and/or inhibits hepatic glucose production.

61. The method of any one of claims 35 to 60, wherein the patient has one or more of an average hemoglobin A1c value of more than about 10% and an average glucose of more than about 200 mg/dl (11 mmol/I) at the start of treatment with conventional diabetic therapy.

62. The method of claim 61, wherein the conventional diabetic therapy is insulin therapy and/or non-insulin diabetes agent therapy.

63. The method of any one of claims 35 to 58, wherein the TNFRSF25 agonistic antibody or antigen binding fragment administration is effective for providing average glucose of below about 200 mg/dl (11 mmol/I) and/or providing average glycosylated hemoglobin levels (hemoglobin A1c) values of about 8% or less.

64. The method of any one of claims 35 to 63, wherein the patient does not experience an increase of insulin production upon TNFRSF25 agonistic antibody or antigen binding fragment administration and/or the TNFRSF25 agonistic antibody or antigen binding fragment administration does not cause one or more of hypoglycemia and hypokalemia.

65. The method of any one of claims 35 to 64, wherein the TNFRSF25 agonistic antibody or antigen binding fragment thereof comprises:
(i) a heavy chain variable region comprising heavy chain CDR1, CDR2, and CDR3 sequences, wherein the heavy chain CDR1 sequence is GFTFSNHDLN (SEQ ID NO: 1), the heavy chain CDR2 sequence is YISSASGLISYADAVRG (SEQ ID NO: 2); and the heavy chain CDR3 sequence is DPAYTGLYALDF (SEQ ID NO: 3) or DPPYSGLYALDF (SEQ ID NO: 4); and
(ii) a light chain variable region comprising light chain CDR1, CDR2, and CDR3 sequences, wherein the light chain CDR1 sequence is TLSSELSWYTIV (SEQ ID NO: 5), the light chain CDR2 sequence is LKSDGSHSKGD (SEQ ID NO: 6), and the light chain CDR3 sequence is CGAGYTLAGQYGWV (SEQ ID NO: 7).

66. The method of claim 65, wherein the TNFRSF25 agonistic antibody or antigen binding fragment thereof further comprises variable region framework (FW) sequences juxtaposed between the CDRs according to the formula (FW1)-(CDR1)-(FW2)-(CDR2)-(FW3)-(CDR3)-(FW4), wherein the variable region FW sequences in the heavy chain variable region are heavy chain variable region FW sequences, and wherein the variable region FW sequences in the light chain variable region are light chain variable region FW sequences.

67. The method of claim 66, wherein the variable region FW sequences are human.

68. The method of any one of claims 1 to 67, wherein the TNFRSF25 agonistic antibody or antigen binding fragment thereof further comprises human heavy chain and light chain constant regions.

69. The method of claim 68, wherein the constant regions are selected from the group consisting of human IgG1, IgG2, IgG3, and IgG4.

70. The method of claim 69, wherein the constant regions are IgG1.

71. The method of claim 69, wherein the constant regions are IgG4.

72. The method of any one of claims 1 to 71, wherein the TNFRSF25 agonistic antibody or antigen binding fragment thereof comprises a heavy chain variable region having the amino acid sequence EVQLVESGGGLSQPGNSLQLSCEASGFTFSNHDLNWVRQAPGKGLEWVAYISSASGLISYADAVRGRFTISRDN AKNSLFLQMNNLKSEDTAMYYCARDPPYSGLYALDFWGQGTQVTVSS (SEQ ID NO: 8), or an amino acid sequence of at least about 85% to about 99% identity thereto.

73. The method of any one of claims 1 to 72, wherein the TNFRSF25 agonistic antibody or antigen binding fragment thereof comprises a light chain variable region having the amino acid sequence QPVLTQSPSASASLSGSVKLTCTLSSELSSYTIVWYQQRPDKAPKYVMYLKSDGSHSKGDGIPDRFSGSSSGAH RYLSISNVQSEDDATYFCGAGYTLAGQYGWVFGSGTKVTVL (SEQ ID NO: 9), or an amino acid sequence of at least about 85% to about 99% identity thereto.

74. The method of any of the above claims, wherein the treatment expand and/or selectively activated a population of Tregs in the patient.

75. A method for treating or preventing graft-versus-host disease (GVHD), comprising administering an effective amount of TNF Receptor Superfamily Member 25 (TNFRSF25) agonistic antibody or antigen binding fragment thereof to a patient in need thereof.

76. The method of claim 75, wherein the patient is a transplant recipient.

77. The method of claim 76, wherein the transplant comprises regulatory T cells (Tregs) from a transplant donor.

78. The method of claim 76 or claim 77, wherein the transplant comprises donor hematopoietic cells.

79. The method of claim 76 or claim 77, wherein the transplant comprises donor stem cells.

80. The method of claim 76 or claim 77, wherein the transplant comprises donor bone marrow cells.

81. The method of claim 76 or claim 77, wherein the transplant comprises islet cells.

82. The method of any one of claims 75 to 81, wherein a graft versus host disease is reduced.

83. The method of claim 82, wherein the graft versus host disease is acute graft-versus-host-disease (aGVHD).

84. The method of any one of claim 82, wherein the graft versus host disease is chronic graft-versus-host-disease (cGVHD).

85. The method of any one of claims 76 to 84, wherein the administration is also to the transplant donor.

86. The method of any one of claims 76 to 85, wherein the administration to the transplant donor occurs prior to transplant.

87. The method of any one of claims 76 to 85, wherein the administration to the transplant recipient occurs after the transplant.

88. The method of any one of claims 76 to 87, wherein the administration is to both the transplant donor and transplant recipient.

89. The method of any one of claims 75 to 88, wherein the TNFRSF25 agonistic antibody or antigen binding fragment causes a sustained increase in Treg cells in the transplant donor and/or transplant recipient.

90. The method of any one of claims 75 to 88, wherein the TNFRSF25 agonistic antibody or antigen binding fragment does not cause substantial Treg suppression in the transplant donor and/or transplant recipient.

91. The method of any one of claims 75 to 88, wherein the TNFRSF25 agonistic antibody or antigen binding fragment does not cause substantial Treg anergy in the transplant donor and/or transplant recipient.

92. The method of claim 75, wherein the method prevents a transplant rejection.

93. The method of claim 92, wherein the method prevents a solid organ transplant rejection.

94. The method of claim 93, wherein the solid organ is selected from lung, kidney, heart, liver, pancreas, thymus, gastrointestinal tract, cornea, eye, and composite allografts.

95. The method of any one of claims 75-94, further comprising administering interleukin-2 (IL-2).

96. The method of claim 95, wherein the IL-2 is a low dose of IL-2.

97. The method of claim 96, wherein the low dose of IL-2 is less than 1 million units per square meter per day.

98. The method of claim 97, wherein the low dose of IL-2 is an amount in the range of about 30,000 to about 300,000 units per square meter per day.

99. The method of claim 96, wherein the low dose of IL-2 is about 300,000 units per square meter per day.

100. The method of claim 96, wherein the low dose of IL-2 is about 30,000 units per square meter per day.

101. The method of claim 96, wherein the administration of low dose IL-2 is sequential with the TNFRSF25 agonistic antibody or antigen binding fragment thereof.

102. The method of claim 96, wherein the administration of low dose IL-2 is concurrent with the TNFRSF25 agonistic antibody or antigen binding fragment thereof.

103. The method of any one of the above claims, wherein the TNFRSF25 agonistic antibody or antigen binding fragment thereof comprises:
(i) a heavy chain variable region comprising heavy chain CDR1, CDR2, and CDR3 sequences, wherein the heavy chain CDR1 sequence is GFTFSNHDLN (SEQ ID NO: 1), the heavy chain CDR2 sequence is YISSASGLISYADAVRG (SEQ ID NO: 2); and the heavy chain CDR3 sequence is DPAYTGLYALDF (SEQ ID NO: 3) or DPPYSGLYALDF (SEQ ID NO: 4); and
(ii) a light chain variable region comprising light chain CDR1, CDR2, and CDR3 sequences, wherein the light chain CDR1 sequence is TLSSELSWYTIV (SEQ ID NO: 5), the light chain CDR2 sequence is LKSDGSHSKGD (SEQ ID NO: 6), and the light chain CDR3 sequence is CGAGYTLAGQYGWV (SEQ ID NO: 7).

104. The method of claim 103, wherein the TNFRSF25 agonistic antibody or antigen binding fragment thereof further comprises variable region framework (FW) sequences juxtaposed between the CDRs according to the formula (FW1)-(CDR1)-(FW2)-(CDR2)-(FW3)-(CDR3)-(FW4), wherein the variable region FW sequences in the heavy chain variable region are heavy chain variable region FW sequences, and wherein the variable region FW sequences in the light chain variable region are light chain variable region FW sequences.

105. The method of claim 103, wherein the variable region FW sequences are human.

106. The method of any one of claims 75-105, wherein the TNFRSF25 agonistic antibody or antigen binding fragment thereof further comprises human heavy chain and light chain constant regions.

107. The method of claim 106, wherein the constant regions are selected from the group consisting of human IgG1, IgG2, IgG3, and IgG4.

108. The method of claim 107, wherein the constant regions are IgG1.

109. The method of claim 107, wherein the constant regions are IgG4.

110. The method of any one of claims 75-109, wherein the TNFRSF25 agonistic antibody or antigen binding fragment thereof comprises a heavy chain variable region having the amino acid sequence EVQLVESGGGLSQPGNSLQLSCEASGFTFSNHDLNWVRQAPGKGLEWVAYISSASGLISYADAVRGRFTISRDN AKNSLFLQMNNLKSEDTAMYYCARDPPYSGLYALDFWGQGTQVTVSS (SEQ ID NO: 8), or an amino acid sequence of at least about 85 to about 99% identity thereto.

111. The method of any one of claims 75 to 110, wherein the TNFRSF25 agonistic antibody or antigen binding fragment thereof comprises a light chain variable region having the amino acid sequence QPVLTQSPSASASLSGSVKLTCTLSSELSSYTIVWYQQRPDKAPKYVMYLKSDGSHSKGDGIPDRFSGSSSGAH RYLSISNVQSEDDATYFCGAGYTLAGQYGWVFGSGTKVTVL (SEQ ID NO: 9), or an amino acid sequence of at least about 85 to about 99% identity thereto.

112. The method of any one of the above claims, further comprising administering an effective amount of TNF Receptor Superfamily Member 25 (TNFRSF25) agonistic antibody or antigen binding fragment thereof, in combination with an immunosuppressant or anti-inflammatory agent, to a patient in need thereof.

113. The method of claim 112, wherein the immunosuppressant or anti-inflammatory agent is an anti-CTLA4 antibody.

114. The method of claim 112, wherein the immunosuppressant or anti-inflammatory agent is an IL-1 receptor antagonist.

115. The method of claim 112, wherein the IL-1 receptor antagonist in KINERET (Anakinra).

116. A method for increasing a graft survival, comprising administering an effective amount of TNF Receptor Superfamily Member 25 (TNFRSF25) agonistic antibody or antigen binding fragment thereof to a patient in need thereof, wherein the patient is a recipient of a graft.

117. The method of claim 116, wherein the patient is a diabetes patient.

118. The method of claim 116 or claim 117, wherein the patient has type 1 diabetes or type 2 diabetes.

119. The method claim 117, wherein the patient has gestational diabetes or steroid-induced diabetes.

120. The method of any one of claims 116 to 119, wherein the graft comprises islet cells.

121. The method of any one of claims 116 to 119, wherein the graft comprises a solid organ.

122. The method of claim 121, wherein the solid organ is selected from pancreas, lung, kidney, heart, liver, thymus, gastrointestinal tract, cornea, eye, and composite allografts.

123. The method of one of claims 116 to 122, wherein the TNFRSF25 agonistic antibody or antigen binding fragment thereof comprises:
(i) a heavy chain variable region comprising heavy chain CDR1, CDR2, and CDR3 sequences, wherein the heavy chain CDR1 sequence is GFTFSNHDLN (SEQ ID NO: 1), the heavy chain CDR2 sequence is YISSASGLISYADAVRG (SEQ ID NO: 2); and the heavy chain CDR3 sequence is DPAYTGLYALDF (SEQ ID NO: 3) or DPPYSGLYALDF (SEQ ID NO: 4); and
(ii) a light chain variable region comprising light chain CDR1, CDR2, and CDR3 sequences, wherein the light chain CDR1 sequence is TLSSELSWYTIV (SEQ ID NO: 5), the light chain CDR2 sequence is LKSDGSHSKGD (SEQ ID NO: 6), and the light chain CDR3 sequence is CGAGYTLAGQYGWV (SEQ ID NO: 7).

124. The method of claim 123, wherein the TNFRSF25 agonistic antibody or antigen binding fragment thereof further comprises variable region framework (FW) sequences juxtaposed between the CDRs according to the formula (FW1)-(CDR1)-(FW2)-(CDR2)-(FW3)-(CDR3)-(FW4), wherein the variable region FW sequences in the heavy chain variable region are heavy chain variable region FW sequences, and wherein the variable region FW sequences in the light chain variable region are light chain variable region FW sequences.

125. The method of claim 124, wherein the variable region FW sequences are human.

126. The method of any one of claims 116 to 125, wherein the TNFRSF25 agonistic antibody or antigen binding fragment thereof further comprises human heavy chain and light chain constant regions.

127. The method of claim 126, wherein the constant regions are selected from the group consisting of human IgG1, IgG2, IgG3, and IgG4.

128. The method of claim 127, wherein the constant regions are IgG1.

129. The method of claim 127, wherein the constant regions are IgG4.

130. The method of any one of claims 116 to 129, wherein the TNFRSF25 agonistic antibody or antigen binding fragment thereof comprises a heavy chain variable region having the amino acid sequence EVQLVESGGGLSQPGNSLQLSCEASGFTFSNHDLNWVRQAPGKGLEWVAYISSASGLISYADAVRGRFTISRDN AKNSLFLQMNNLKSEDTAMYYCARDPPYSGLYALDFWGQGTQVTVSS (SEQ ID NO: 8), or an amino acid sequence of at least about 85 to about 99% identity thereto.

131. The method of any one of claims 116 to 130, wherein the TNFRSF25 agonistic antibody or antigen binding fragment thereof comprises a light chain variable region having the amino acid sequence QPVLTQSPSASASLSGSVKLTCTLSSELSSYTIVWYQQRPDKAPKYVMYLKSDGSHSKGDGIPDRFSGSSSGAH RYLSISNVQSEDDATYFCGAGYTLAGQYGWVFGSGTKVTVL (SEQ ID NO: 9), or an amino acid sequence of at least about 85 to about 99% identity thereto.
